(19) United States
(12) Patent Application Publication
(10) Pub. No.: US 2016/0322585 A1
(43) Pub. Date: Nov. 3, 2016

(54) CONDENSED CYCLIC COMPOUND AND ORGANIC LIGHT EMITTING DEVICE INCLUDING THE SAME

(71) Applicants: SAMSUNG SDI CO., LTD., Yongin-si (KR); SAMSUNG ELECTRONICS CO., LTD., Suwon-si (KR)

(72) Inventors: Chang-Woo KIM, Suwon-si (KR); Byung-Ku KIM, Suwon-si (KR); Young-Kwon KIM, Suwon-si (KR); Hyung-Sun KIM, Suwon-si (KR); Joo-Hee SEO, Suwon-si (KR); Chang-Ju SHIN, Suwon-si (KR); Seung-Jae LEE, Suwon-si (KR); Kyu Young HWANG, Ansan-si (KR)

(73) Assignees: Samsung SDI Co., LTD., Yongin-si, Gyeonggi-do (KR); Samsung Electronics Co., Ltd., Suwon-si, Gyeonggi-do (KR)

(21) Appl. No.: 15/104,017

(22) PCT Filed: Feb. 9, 2015
(86) PCT No.: PCT/KR2015/001286

§ 371 Date: Jun. 13, 2016

(30) Foreign Application Priority Data
Mar. 10, 2014 (KR) 10-2014-0027948
Feb. 9, 2015 (KR) 10-2015-0019192

(51) Int. Cl. H01L 51/00; C07D 495/04; C07D 491/048; C09K 11/02

(52) U.S. Cl. CPC H01L 51/0072; C09K 11/025; C07D 495/04; H01L 51/0071; C07D 491/048; H01L 2251/5384; H01L 51/5012

(57) ABSTRACT

A novel condensed cyclic compound and an organic light emitting device including the same are disclosed.

### TECHNICAL FIELD

A condensed cyclic compound and an organic light emitting device including the same are disclosed.

### BACKGROUND ART

Organic Light emitting devices (OLEDs), which are self-emitting devices, have advantages such as wide viewing angles, excellent contrast, quick response, high brightness, excellent driving voltage characteristics, and may provide multicolored images. For example, an organic light emitting device includes an anode, a cathode, an emission layer interposed between the anode and the cathode. The organic light emitting device may include a hole transport region between the anode and the emission layer, and an electron transport region between the emission layer and the cathode. Holes injected from the anode move to the emission layer via the hole transport region, white electrons injected from the cathode move to the emission layer via the electron transport region. Carriers such as the holes and electrons recombine in the emission layer to generate excitons. When the excitons drop from an excited state to a ground state, light is emitted.

### DISCLOSURE

### Technical Problem

A novel condensed cyclic compound and an organic light emitting device including the same are provided.

### Technical Solution

According to one aspect, a condensed cyclic compound represented by the following Formula 1 is provided:

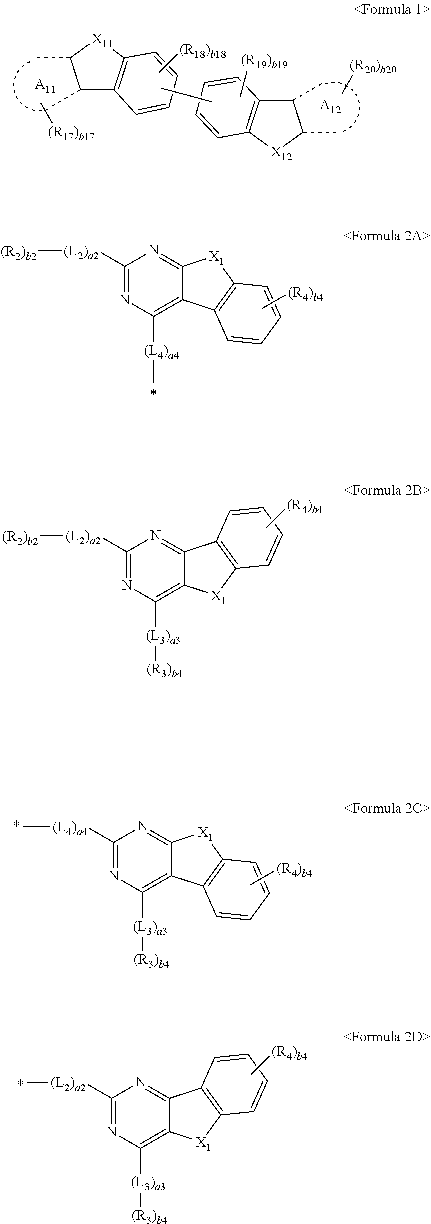

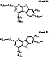

wherein, in the formulae,

ring A₁₁ and ring A₁₂ are independently selected from benzene, naphthalene, pyridine, pyrimidine, pyrazine, pyridazine, and triazine;

X₁₁ is selected from N-[(L₁₁)ₐ₁₁-(R₁₁)_{b11}], S, O, (S═O), S(═O)₂, C(═O), C(R₁₃)(R₁₄), Si(R₁₃)(R₁₄), P(R₁₃), P(═O)(R₁₃), and C═N(R₁₃);

X₁₂ is selected from N-[(L₁₂)ₐ₁₂-(R₁₂)_{b12}], S, O, S(═O), S(═O)₂, C(═O), C(R₁₅)(R₁₆), Si(R₁₅)(R₁₆), P(R₁₅), P(═O)(R₁₅), and C═N(R₁₅);

X₁ is selected from N-[(L₁)ₐ₁-(R₁)_{b1}], S, O, S(═O), S(═O)₂, C(═O), Si(R₅)(R₆), P(R₅), P(═O)(R₅), and C═N(R₅);

L₁ to L₄, L₁₁ and L₁₂ are independently selected from a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylenylene group, a substituted or unsubstituted C₂-C₁₀ heterocycloalkenylene group, a substituted r unsubstituted C₆-C₆₀ arylene group, a substituted or unsubstituted C₂-C₆₀ heteroarylene group, a substituted or unsubstituted divalent non-aromatic condensed polycyclic group, and a substituted or unsubstituted divalent non-aromatic heterocondensed polycyclic group;

a1 to a4, a11 and a12 are independently an integer selected from 0 to 3;

R₁ to R₆ and R₁₁ to R₂₀ are independently selected from hydrogen, deuterium, —F (a fluoro group), —Cl (a chloro group), —Br (a bromo group), —I (an iodo group), a hydroxyl group, a cyano group, a nitro group, an amino group, amidino group, a hydrazine group, a hydrazone group, a carboxylic acid or a salt thereof, a sulfonic add or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic heterocondensed polycyclic group, —N(Q₁)(Q₂), —Si(Q₃)(Q₄)(Q₅), —B(Q₆)(Q₇), and the group represented by the Formula 2A to the group represented by the Formula 2F, provided that the R₁ to R₆ are not the group represented by the Formula 2A to the group represented by the Formula 2F;

b1 to b4, b11, b12 and b16 to b20 are independently an integer selected from 1 to 3;

i) in Formula 1, when X₁₁ is N-[(L₁₁)ₐ₁₁-(R₁₁)_{b11}] and X₁₂ is not N-[(L₁₂)ₐ₁₂-(R₁₂)_{b12}], in Formula 1 at least one of b17 R₁₇'s, b11 R₁₁'s, b18 R₁₈'s, b19 R₁₉'s, and b20 R₂₀'s is selected from the group represented by the Formula 2A to the group represented by the Formula 2 F;

ii) in Formula 1; when X₁₁ is not N-[(L₁₁)ₐ₁₁-(R₁₁)_{b11}] and X₁₂ is not N-[(L₁₂)ₐ₁₂-(R₁₂)_{b12}], in Formula 1, at least one of b17 R₁₇'s, b11 R₁₁'s, b18 R₁₈'s, b19 R₁₉'s, and b20 R₂₀'s is selected from the group represented by the Formula 2A to the group represented by the Formula 2F;

iii) in Formula 1, when X₁₁ is N-[(L₁₁)ₐ₁₁-(R₁₁)_{b11}], and X₁₂ is not N-[(L₁₂)ₐ₁₂-(R₁₂)_{b12}], in Formula 1, as least one of b17 R₁₇'s, b11 R₁₁'s, b18 R₁₈'s, b19 R₁₉'s, and b20 R₂₀'s is selected from the group represented by the Formula 2A to the group represented by the Formula 2F;

at least one substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀arylene group, the substituted C₂-C₆₀ heteroarylene group, the substituted divalent non-aromatic condensed polycyclic group, the substituted divalent non-aromatic heterocondensed polycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic heterocondensed polycyclic group is selected from

deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group and a C₁-C₆₀ alkoxy group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic heterocondensed polycyclic group, —N(Q₁₁)(Q₁₂), —Si(Q₁₃)(Q₁₄)(Q₁₅) and —B(Q₁₆)(Q₁₇);

a C₃-C₁₀ cycloalkyl group; a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic heterocondensed polycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇);

wherein the Q₁ to Q₇, Q₁₁ to Q₁₇, Q₂₁ to Q₂₇ and Q₃₁ to Q₃₇ are independently selected from hydrogen, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group; a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic

According to another aspect, an organic light emitting device includes a first electrode; a second electrode, and an organic layer including an emission layer interposed between the first electrode and the second electrode, and the organic layer includes at least one of the condensed cyclic compounds represented by the Formula 1.

The condensed cyclic compound may be included in the emission layer, the emission layer may further include a dopant, and the condensed cyclic compound in the emission layer may function as a host.

### Advantageous Effects

The condensed cyclic compound has excellent electrical characteristics and thermal stability, and thus an organic light emitting device including the condensed cyclic compound may have a low driving voltage, high efficiency, high luminance and long life-span characteristics.

### DESCRIPTION OF THE DRAWINGS

FIG. 1 is a schematic cross-sectional view showing an organic light emitting device according to one embodiment.

### MODE FOR INVENTION

The condensed cyclic compound is represented by the following Formula 1:

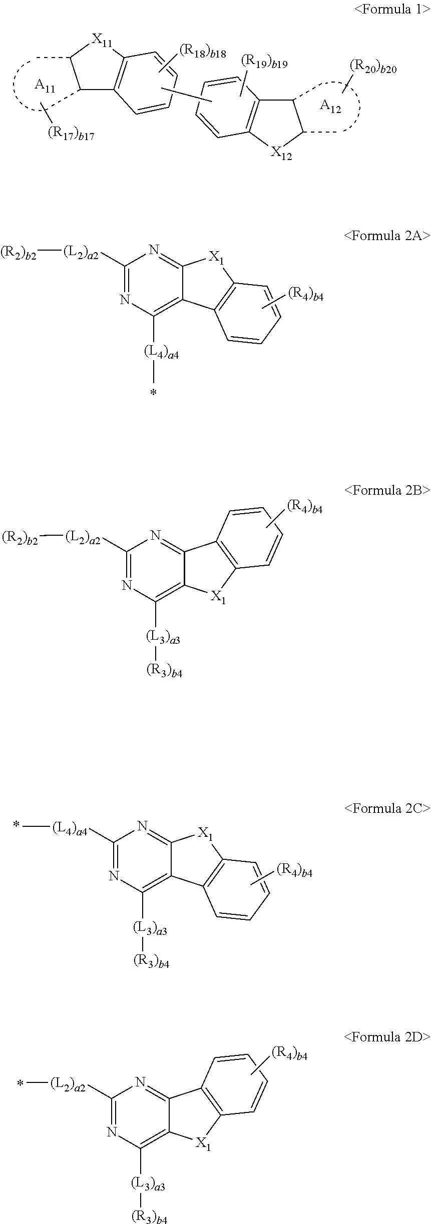

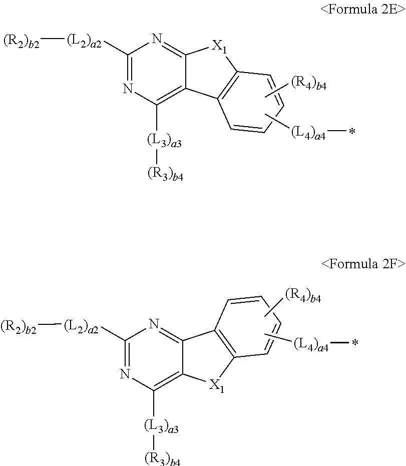

In Formula 1, the ring A₁₁ and ring A₁₂ are independently selected from benzene, naphthalene, pyridine, pyrimidine, pyrazine, pyridazine, and triazine.

For example, the ring A₁₁ and ring A₁₂ may be benzene, but are not limited thereto.

In Formula 1, X₁₁ is selected from N-[(L₁₁)ₐ₁₁-(R₁₁)_{b11}], S, O, S(═O), S(═O)₂, C(═O), C(R₁₃)(R₁₄), Si(R₁₃)(R₁₄), P(R₁₃), P(═O)R₁₃ and C═N(R₁₃), in Formula 1B, X₁₂ is selected from N-[(L₁₂)ₐ₁₂-(R₁₂)_{b12}], S, O, S(═O), S(═O)₂, C(═O), C(R₁₅)(R₁₆), Si(R₁₅)(R₁₆), P(R₁₅), P(═O)R₁₅ and C═N(R₁₅), and in Formulae 2A to 2F, X₁ is selected from N-[(L₁)ₐ₁-(R₁)_{b1}], S, O, S(═O), S(═O)₂, C(═O), C(R₁₅)(R₁₆), P(R₅), P(═O)R₅ and C═N(R₅).

The relationship between the Formula 1 and the Formulae 2A to 2F are as follows:

i) in Formula 1, when X₁₁ is N-[(L₁₁)ₐ₁₁-(R₁₁)_{b11}] and X₁₂ is not N-[(L₁₂)ₐ₁₂-(R₁₂)_{b12}], in Formula 1, at least one of b17 R₁₇'s, b11 R₁₁'s, B18 R₁₈'s, b19 R₁₉'s, and b20 R₂₀'s is selected from the group represented by the Formula 2A to the group represented by the Formula 2F;

ii) in Formula 1, when X₁₁ is not N-[(L₁₁)ₐ₁₁-(R₁₁)_{b11}] and X₁₂ is N-[(L₁₂)ₐ₁₂-(R₁₂)_{b12}], in Formula 1, at least one of b17 R₁₇'s, b11 R₁₁'s, b18 R₁₈'s, b19 R₁₉'s, and b20 R₂₀'s is selected from the group represented by the Formula 2A to the group represented by the Formula 2F; and

iii) in Formula 1, when X₁₁ is N-[(L₁₁)ₐ₁₁-(R₁₁)_{b11}] and X₁₂ is N-[(L₁₂)ₐ₁₂-(R₁₂)_{b12}], in Formula 1, at least one of b17 R₁₇'s, b11 R₁₁'s, b18 R₁₈'s, b19 R₁₉'s, b12 R₁₂'s, and b20 R₂₀'s is selected from the group represented by the Formula 2A to the group represented by the Formula 2F.

According to one embodiment, X₁₁ in Formula 1 may be selected from N-[(L₁₁)ₐ₁₁-(R₁₁)_{b11}], S, and O.

According to another embodiment, X₁₂ in Formula 1B may be selected from

For example, X₁₁ may be N-[(L₁₁)ₐ₁₁-(R₁₁)_{b11}], and X₁₂ may be N-[(L₁₂)ₐ₁₂-(R₁₂)_{b12}], but is not limited thereto.

According to another embodiment, X₁ in Formulae 2A to 2F may be N-[(L₁)ₐ₁-(R₁)_{b1}], S, and O.

In the formulae, L₁ to L₄, L₁₁ and L₁₂ may be independently selected from a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₂-C₆₀ heteroarylene group, a substituted or unsubstituted divalent non-aromatic condensed polycyclic group, or a substituted or unsubstituted divalent non-aromatic heterocondensed polycyclic group.

For example, in the formulae, L₁ to L₄, L₁₁ and L₁₂ may be independently selected from

a phenylene group, a pentalenylene group, an indenylene group, a naphthylene group, an azulenylene group, a heptalenylene group, an indacenylene group, an acenaphthylene group, a fluorenylene group, a spiro-fluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, a pentaphenylene group, hexacenylene group, a pyrrolylene group, an imadazolylene group, a pyrazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzooxazolylene group, a benzoimidazolylene group, a furanylene group, a benzofuranylene group, a thiophenylene group, a benzothiophenylene group, s thiazolylene group, an isothiazolylene group, a benzothiazolylene group, an isoxazolylene group, an oxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, an imidazopyrimidinylene group, and an imidazopyridinylene group; and

a phenylene group, a pentalenylene group, an indenylene group, a naphthylene group, an azulenylene group, a heptalenylene group, an indacenylene group, an acenaphthylene group, a fluorenylene group, a spiro-fluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, pentaphenylene group, a hexacenylene group, a pyrrolylene group, an imidazolylene group, a pyrazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzooxazolylene group, a benzoimidazolylene group, a furanylene group, a benzofuranylene group, a thiophenylene group, a benzothiophenylene group, a thiazolylene group, an isothiazolylene group, a benzothiazolylene group, an isoxazolylene group, an oxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, an imidazopyrimidinylene group and an imidazopyridinylene group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₆-C₂₀ aryl group, a C₂-C₆₀ heteroaryl group, a monovalent non-aromatic condensed polycyclic group, a monovalent non-aromatic heterocondensed ring group, and —Si(Q₃₃)(Q₃₄)(Q₃₅); and

wherein Q₃₃ to Q₃₅ are independently selected from hydrogen, a C₁-C₂₀alkyl group, a C₁-C₂₀alkoxy group, a phenyl group, a naphthyl group, an anthracenyl group, a pyrenyl group, a phenanthrenyl group, a fluorenyl group, a carbazolyl group, a benzocarbazolyl group, a dibenzocarbazolyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group or a quinazolinyl group.

According to one embodiment, in the formulae, L₁ to L₄, L₁₁ to L₁₂ may be independently selected from one of Formulae 2-1 to 2-13, but are not limited thereto:

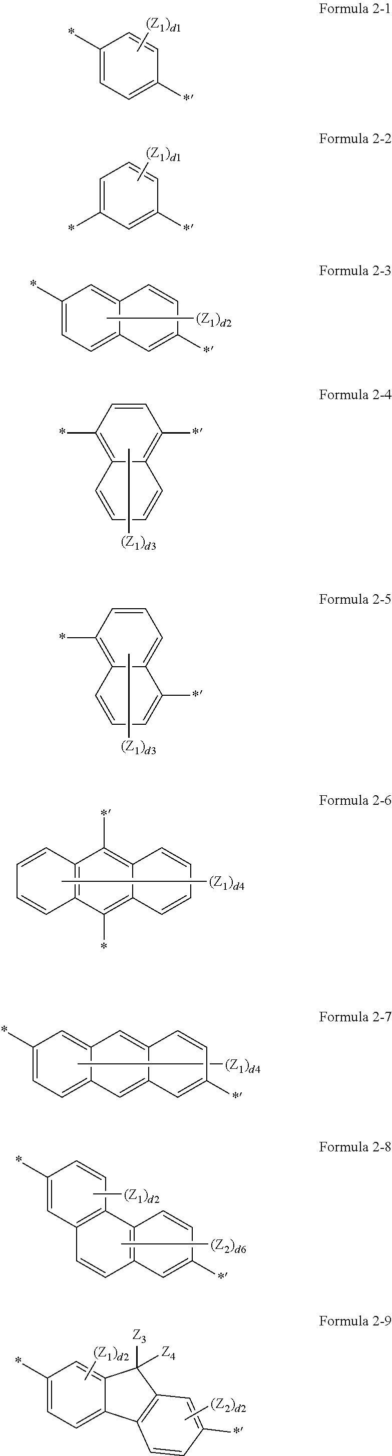

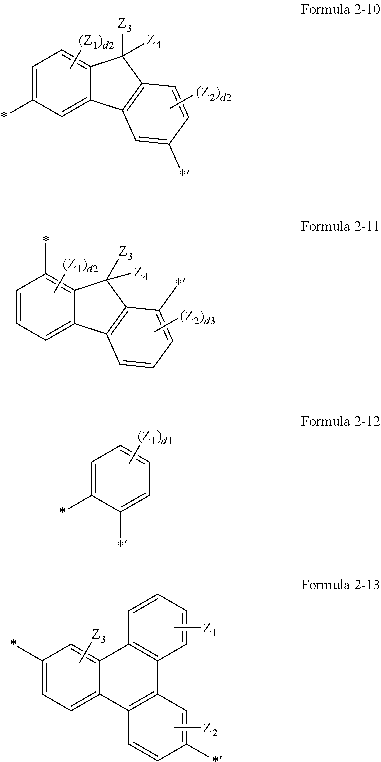

In Formulae 2-1 to 2-13,

Z₁ to Z₄ are independently selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, an anthracenyl group, a triphenylenyl group, a pyrenyl group, a phenanthrenyl group, a fluorenyl group, a chrysenyl group, a carbazolyl group, a benzocarbazolyl group, a dibenzocarbazolyl group, a dibenzofuranyl group, a dibenzothiophenyl group, a pyridinyl group, a pyrimidinyl group, a trizinyl group, a quinolinyl group, an isoquinolinyl group, a quinazolinyl group, a quinoxalinyl group, a biphenyl group and —Si(Q₃₃)(Q₃₄)(Q₃₅);

wherein the Q₃₃ to Q₃₅ are independently selected from hydrogen, a C₁-C₂₀alkyl group, a C₁-C₂₀ alkoxy group, a phenyl group, a naphthyl group, an anthracenyl group, a pyrenyl group, a phenanthrenyl group, a fluorenyl group, a chrysenyl group, a carbazolyl group, a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group and a quinoxalinyl group;

d1 is selected from integers of 1 to 4, d2 is selected from integers of 1 to 3, d3 is selected from integers of 1 to 6, d4 is selected from integers of 1 to 8, d6 is selected from integers of 1 to 5, and * and *′ indicate bonding sites with neighboring atoms.

According to another embodiment, in the formulae, L₁ to L₄, L₁₁ and L₁₂ may be independently selected from the following Formulae 3-1 to 3-12, but are not limited thereto:

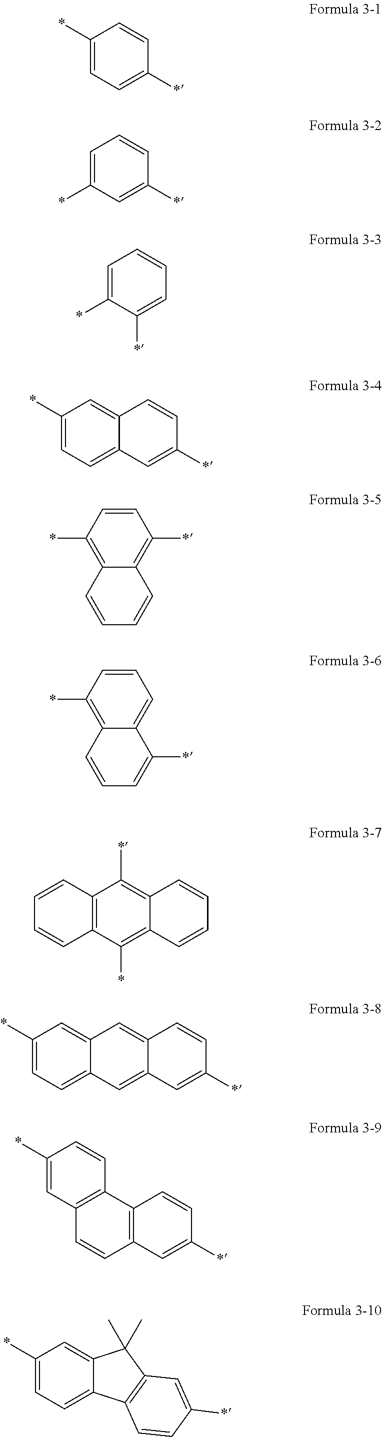

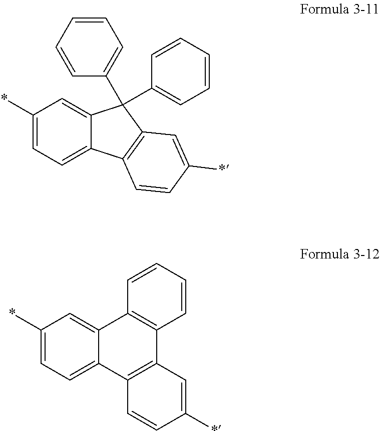

In Formulae 2A to 2F, a1 indicates the number of L₁, and selected from integers of 0 to 3. For example, a1 may be 0 or 1. When a1 is 0, R₁ is directly linked to N. When a1 is greater than or equal to 2, two or more L₁ may be the same or different from each other. The descriptions of a2 to a4, a11 and a12 refer to the descriptions of the a1 and Formula 1 and Formulae 2A to 2F.

According to one embodiment, a1 to a4, a11 and a12 may be independently 0 or 1.

Herein, in the formulae, R₁ to R₆ and R₁₁ to R₂₀ are independently selected from hydrogen, deuterium, —F (a fluoro group), —Cl (a chloro group), —Br (a bromo group), —I (an iodo group), a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀arylthio group, a substituted or unsubstituted C₂-C₆₀ heteroaryl group, a substituted or unsubstituted monovalent non-aromatic condensed polycyclic group, a substituted or unsubstituted monovalent non-aromatic heterocondensed polycyclic group, —N(Q₁)(Q₂), —Si(Q₃)(Q₄)(Q₅), —B(Q₆)(Q₇), and the group represented by the Formula 2A to the group represented by the Formula 2F, provided that the R₁ to R₆ are not the group represented by the Formula 2A to the group represented by the Formula 2F.

According to one embodiment, the R₁ to R₆ and R₁₁ to R₂₀ are independently selected from

hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group and a C₁-C₂₀ alkoxy group;

a C₁-C₂₀ alkyl group and a C₁-C₂₀ alkoxy group substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, and a phosphoric acid or a salt thereof;

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyranyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a trizolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;

a phenyl group, a pentalenyl group, an indenyl group, a naphthyl group, a azulenyl group, a heptalenyl group, an indacenyl group, an acenaphthyl group, a fluoranyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl, a benzoimidazolyl group, a benzofuranyl group, a benzothiophenyl group, an isobenzothiazolyl group, a benzooxazolyl group, an isobenzooxazolyl group, a trizolyl group, a tetrazolyl group, an oxadiazolyl group, a trizinyl group, a dibenzofuranyl group, a dibenzothiophenyl group, a benzocarbazolyl group, a dibenzocarbazolyl group, an imidazopyridinyl group and an imidazopyrimidinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an arthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, and a biphenyl group;

—Si(Q₃)(Q₄)(Q₅) (provided that, the R₁₃ to R₁₆ and R₅ and R₆ are not —Si(Q₃)(Q₄)(Q₅)); and

the group represented by the Formula 2A to the group represented by the Formula 2F (provided that the R₁ to R₆ are not the group represented by the Formula 2A to the group represented by the Formula 2F);

wherein the Q₃ to Q₅ and Q₃₃ to Q₃₅ are independently selected from hydrogen, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a chrysenyl group, a carbazolyl group, a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group and a quinoxalinyl group, but are not limited thereto.

According to another embodiment, in the formulae, R₁ to R₆, R₁₁ to R₁₆ and R₂₁ to R₃₀ are independently selected from

hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof C₁-C₂₀alkyl group and C₁-C₂₀ alkoxy group;

a C₁-C₂₀ alkyl group and a C₁-C₂₀ alkoxy group substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, and a phosphoric acid or a salt thereof;

the following Formulae 4-1 to 4-31;

—Si(Q₃)(Q₄)(Q₅) (provided that the R₁₃ to R₁₆ and R₅ to R₆ are not —Si(Q₃)(Q₄)(Q₅)); and

the group represented by the Formula 2A to the group represented by the Formula 2F (provided that R₁ to R₆ are not the group represented by the Formula 2A to the group represented by the Formula 2F), but are not limited thereto;

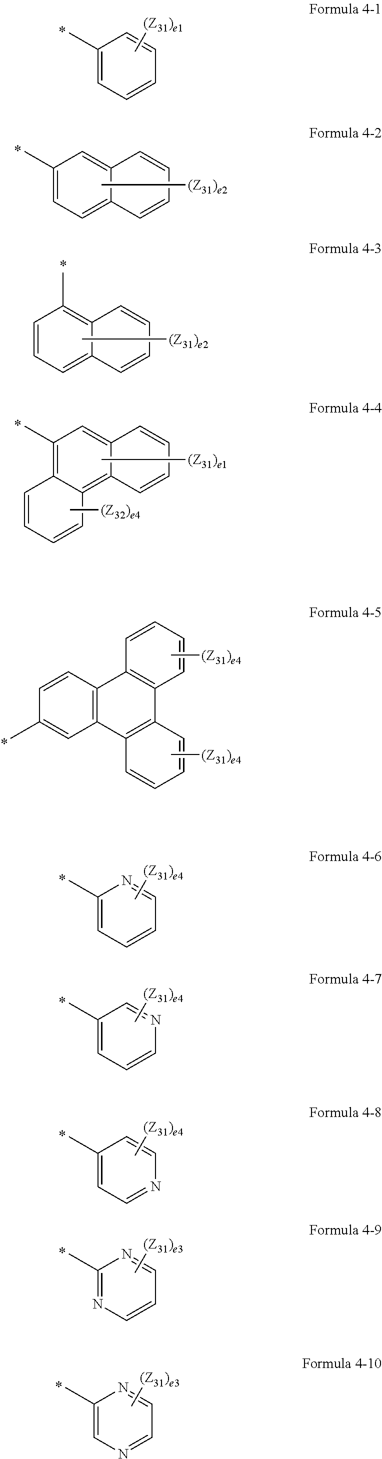

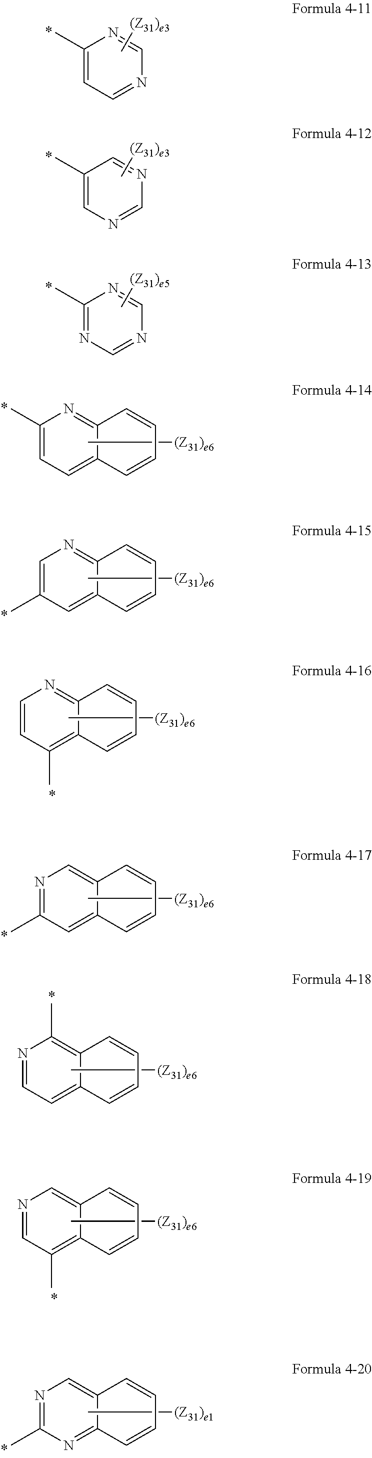

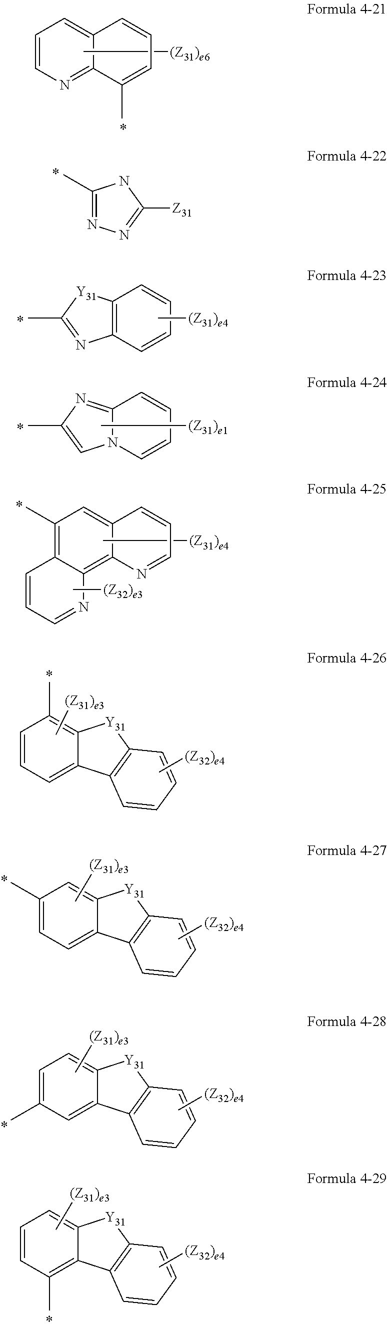

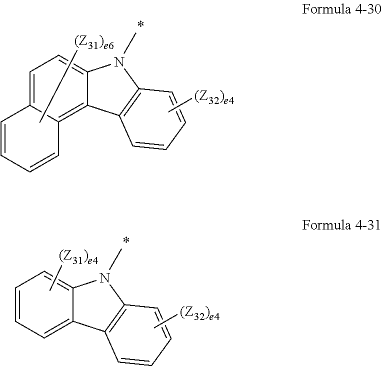

In Formulae 4-1 to 4-31.

Y₃₁ is O, S, C(Z₃₃)(Z₃₄), N(Z₃₅) or Si(Z₃₆)(Z₃₇) (provided that, in Formula 4-23, Y₃₁ is not NH);

Z₃₁ to Z₃₇ are independently selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, an anthracenyl group, a triphenylenyl group, a pyrenyl group, a phenanthrenyl group, a fluorenyl group, a chrysenyl group, a carbazolyl group, a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group, a quinoxalinyl group, a biphenyl group, and —Si(Q₃₃)(Q₃₄)(Q₃₅);

wherein the Q₃ to Q₅ and Q₃₃ to Q₃₅ are independently selected from hydrogen, a C₁-C₂₀ alkyl group, a C₁-C₂₀ alkoxy group, a phenyl group, a naphthyl group, an anthracenyl group, a triphenylenyl group, a pyrenyl group, a phenanthrenyl group, a fluorenyl group, a chrysenyl group, a carbazolyl group, a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group and a quinoxalinyl group;

e1 is selected from integers of 1 to 5, e2 is selected from integers of 1 to 7, e3 is selected from integers of 1 to 3, e4 is selected from integers of 1 to 4, e5 is 1 or 2, e6 is selected from integers of 1 to 6, and * indicates a bonding site with a neighboring atom.

According to another embodiment, in the formulae, R₁ to R₆ and R₁₁ to R₂₀ are independently selected from

hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and a C₁-C₂₀ alkoxy group;

a C₁-C₂₀ alkyl group and a C₁-C₂₀ alkoxy group substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, and a phosphoric acid or a salt thereof;

the following Formulae 5-1 to 5-63;

—Si(Q₃)(Q₄)(Q₅ (provided that the R₁₃ to R₁₆, R₅ and R₆ are not —Si(Q₃)(Q₄)(Q₅)); and

the group represented by the Formula 2A to the group represented by the Formula 2F (provided that the R₁ to R₆ are not the group represented by the Formula 2A to the group represented by the Formula 2F); but are not limited thereto:

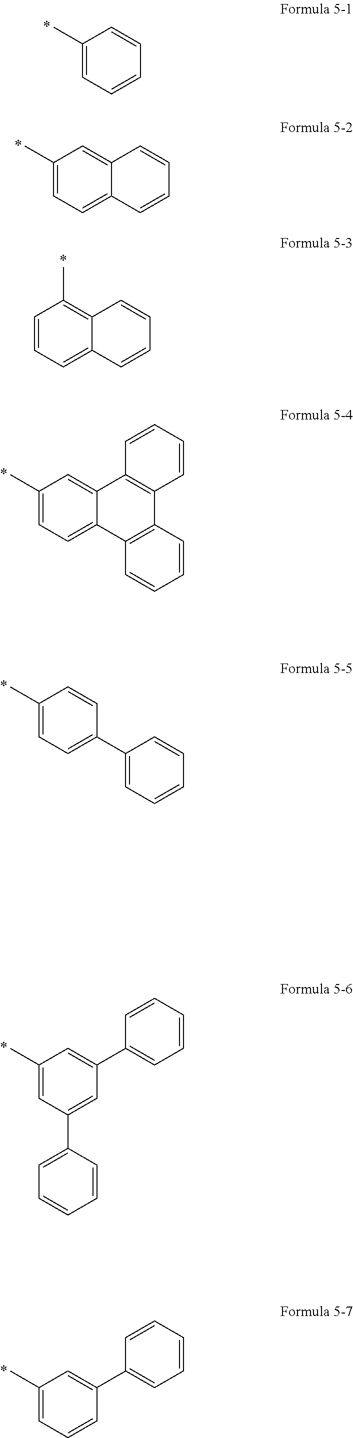

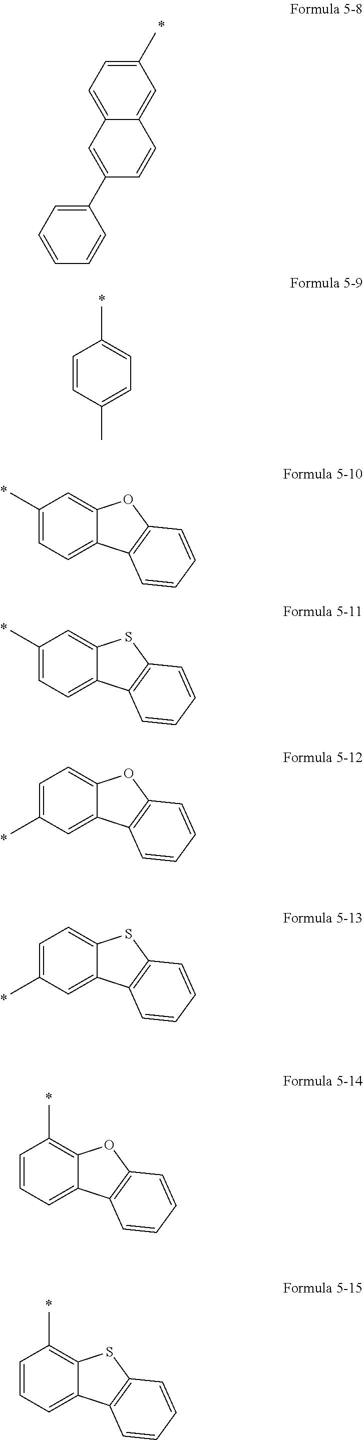

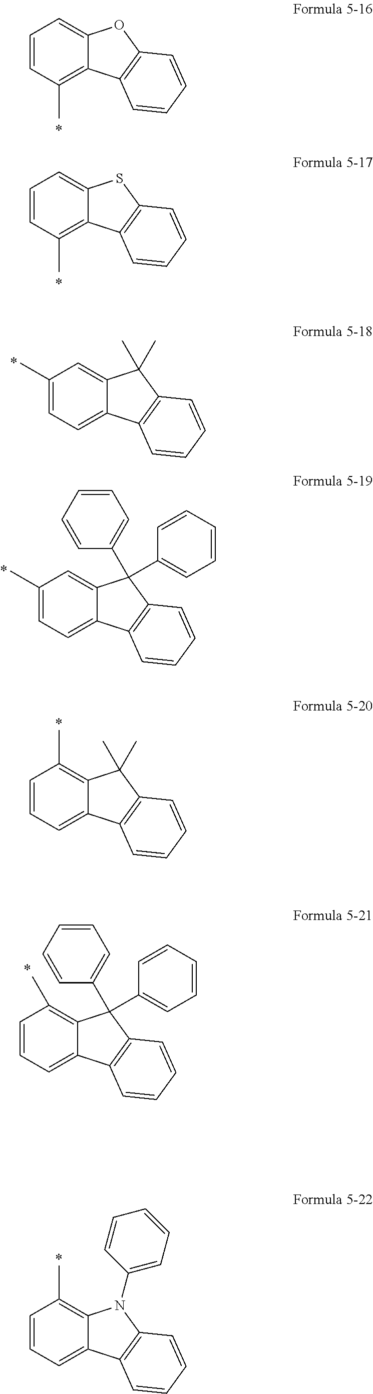

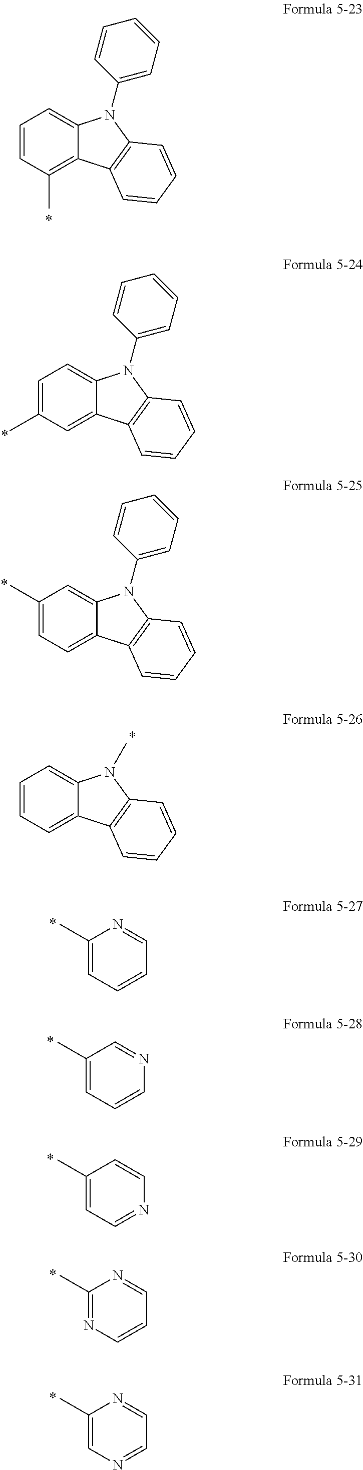

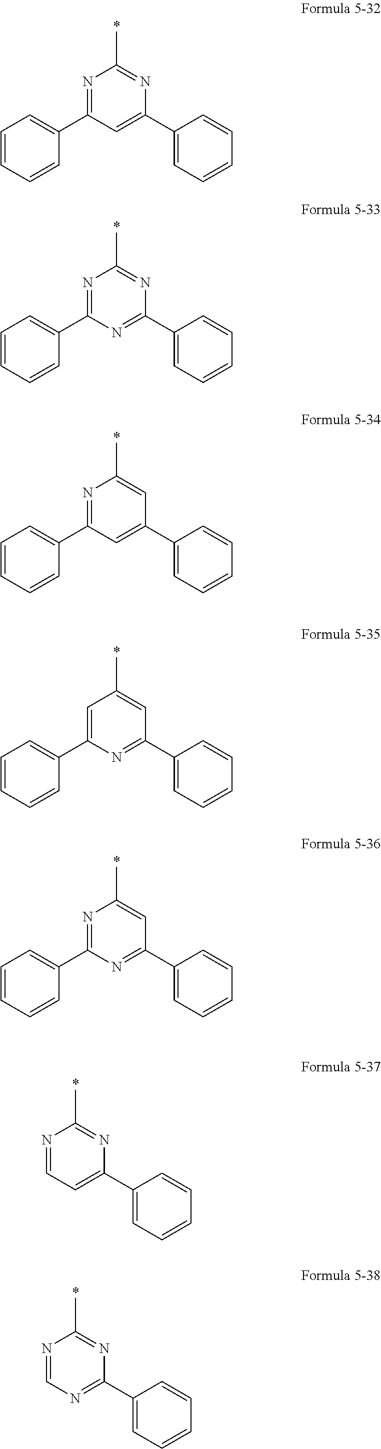

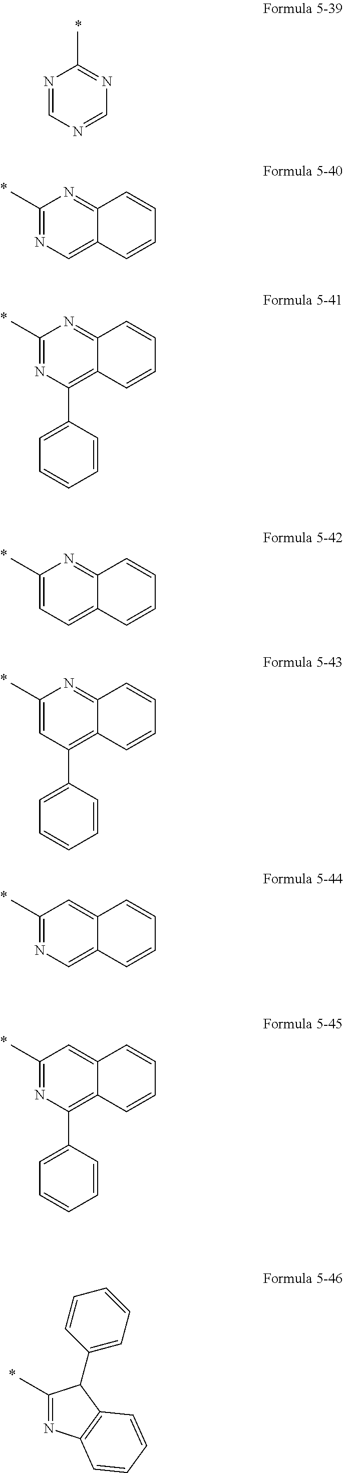

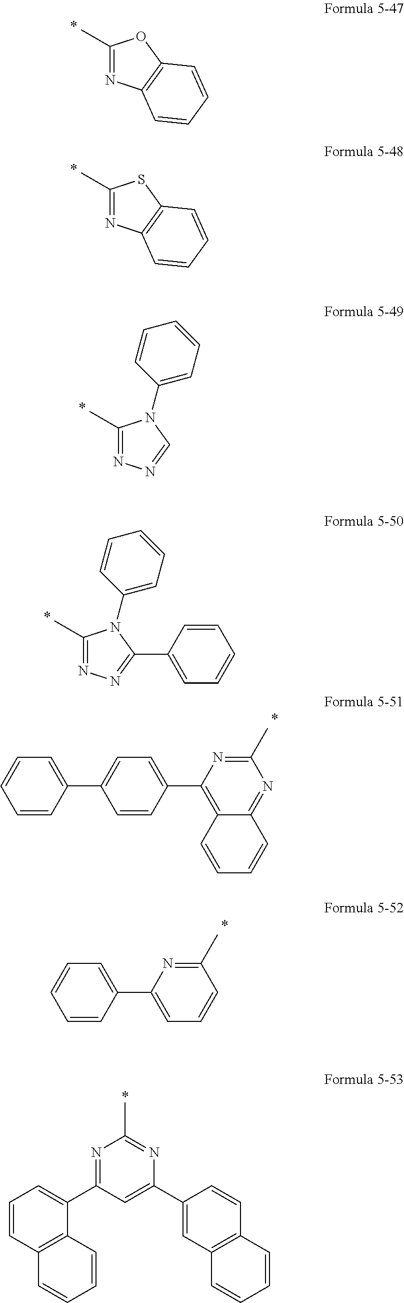

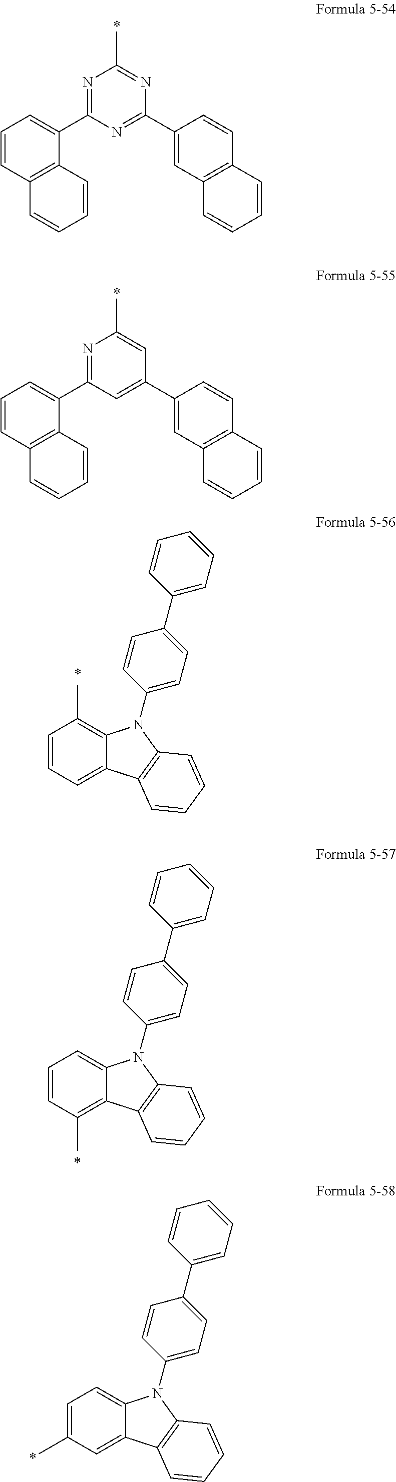 [SMILES: Cc1nc(-c2ccc3ccccc3c2)nc(-c2cccc3ccccc23)n1 ; Cc1cc(-c2ccc3ccccc3c2)cc(-c2cccc3ccccc23)n1 ; Cc1cccc2c3ccccc3n(-c3ccc(-c4ccccc4)cc3)c12 ; Cc1cccc2c1c1ccccc1n2-c1ccc(-c2ccccc2)cc1 ; Cc1ccc2c(c1)c1ccccc1n2-c1ccc(-c2ccccc2)cc1]

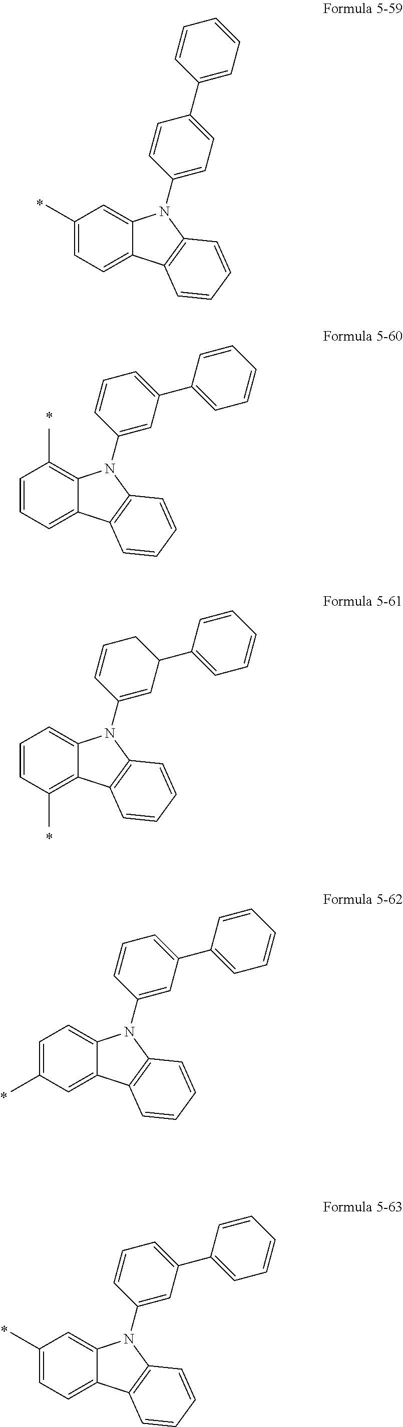 [SMILES: Cc1ccc2c3ccccc3n(-c3ccc(-c4ccccc4)cc3)c2c1 ; Cc1cccc2c3ccccc3n(-c3cccc(-c4ccccc4)c3)c12 ; Cc1cccc2c1c1ccccc1n2C1=CC(c2ccccc2)CC=C1 ; Cc1ccc2c(c1)c1ccccc1n2-c1cccc(-c2ccccc2)c1 ; Cc1ccc2c3ccccc3n(-c3cccc(-c4ccccc4)c3)c2c1]

On the other hand, R₂ in Formulae 2A and 26, R₃ in Formulae 2C and 2D and R₂ and R₃ in Formulae 2E and 2F are independently selected from,

deuterium, —F, —Cl, —Br, —I, a hydroxyl group, a cyano group, a nitro group, an amino group, an amidino group, a hydrazine group, a hydrazone group, a a carboxylic acid or a salt thereof, a sulfonic acid or a salt thereof, a phosphoric acid or a salt thereof, a C₁-C₂₀ alkyl group and a C₁-C₂₀ alkoxy group;

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group or an imidazopyrimidinyl group; and

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group or an imidazopyrimidinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, and a biphenyl group;

wherein the Q₃₃ to Q₃₅ are independently selected from hydrogen, a C₁-C₂₀ alkyl group, a C₁-C₂₀ alkoxy group, a phenyl group, a naphthyl group, an anthracenyl group, pyrenyl group, a phenanthrenyl group, a fluorenyl group, a chrysenyl group, a carbazolyl group; a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group and a quinoxalinyl group.

According to one embodiment, R₂ in Formula 2A and 2B, R₃ in Formulae 2C and 2D, and at least one of R₂ and R₃ in Formulae 2E and 2F is,

a phenyl group, a pentalenyl group, an indenyl group, a naphthyl group, an azulenyl group, a heptalenyl group, an indacenyl group, an acenaphthyl group, a fluoranyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a napthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl groups or an imidazopyrimidinyl group; and

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group or an imidazopyrimidinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, and a biphenyl group;

wherein the Q₃₃ to Q₃₅ are independently selected from hydrogen, a C₁-C₂₀ alkyl group, a C₁-C₂₀ alkoxy group, a phenyl group, a naphthyl group, an anthracenyl group, pyrenyl group, a phenanthrenyl group, a fluorenyl group, a chrysenyl group, a carbazolyl group, a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group and a quinoxalinyl group.

For example, R₂ in Formula 2A and 2B, R₃ in Formulae 2C and 2D, and at least one of R₂ and R₃ in Formulae 2E and 2F is selected from,

a phenyl group, a naphthyl group, a triphenylenyl group, a phenanthrenyl group, a pyridinyl group, a pyrimidinyl group, a pyrazinyl group, a pyridazinyl group, or a triazinyl group; and

a phenyl group, a naphthyl group, a triphenylenyl group, a phenanthrenyl group, a pyridinyl group, a pyrimidinyl group, a pyrazinyl group, a pyridazinyl group, or a triazinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a naphthyl group, a triphenylenyl group, a phenanthrenyl group, a pyridinyl group, a pyrimidinyl group, a pyrazinyl group, a pyridazinyl group, and a trizinyl group;

wherein the Q₃₃ and Q₃₅ are independently selected from hydrogen, a C₁-C₂₀ alkyl group, a C₁-C₂₀ alkoxy group, a phenyl group, and a naphthyl group.

According to another embodiment, R₂ in Formulae 2A and 2B, R₃ in Formulae 2C and 2D and at least one of R₂ and R₃ in Formulae 2E and 2F may be selected from the Formulae 5-1 to 5-63, but are not limited thereto.

According to another embodiment, R₁ of X₁ in Formulae 2A to 2F, R₁₁ of X₁₁ in Formula 1, and R₁₂ of X₁₂ in Formula 1B are independently selected from a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₂-C₂₀ heteroaryl group, a substituted or unsubstituted monovalent non-aromatic condensed polycyclic group, a substituted or unsubstituted monovalent non-aromatic heterocondensed polycyclic group, and the group represented by the Formula 2A to the group represented by the Formula 2F, provided that the R₁ is not the group represented by the Formula 2A to the group represented by the Formula 2F.

For example, R₁ of X₁ in Formulae 2A to 2F, R₁₁ of X₁₁ in Formula 1, and R₁₂ of X₁₂ in Formula 1B are independently selected from

a phenyl group, a naphthyl group, a triphenylenyl group, a phenanthrenyl group, a pyridinyl group, a pyrimidinyl group, a pyrazinyl group, a pyridazinyl group, or a triazinyl group;

a phenyl group, a naphthyl group, a triphenylenyl group, a phenanthrenyl group, a pyridinyl group, a pyrimidinyl group, a pyrazinyl group, a pyridazinyl group, and a triazinyl group, each of which is substituted with at least one of deuterium, —F, —Cl, —Br, —I, a hydroxyl group, a cyano group, a nitro group, an amino group, an amidino group, a hydrazine group, a hydrazone: group, a carboxylic acid or a salt, thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a naphthyl group, a triphenylenyl group, a phenanthrenyl group, pyridinyl group, pyrimidinyl group, a pyrazinyl group, a pyridazinyl group, and a triazinyl group; and

the group represented by the Formula 2A to the group represented by the Formula 2F (R₁ is not the group represented by the Formula 2A to the group represented by the Formula 2F), but are not limited thereto.

According to another embodiment, R₄ in Formulae 2A to 2F may be independently selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group and a C₁-C₂₀ alkoxy group, but is not limited thereto.

In the Formulae, b1 indicates the number of R₁, and may be selected from integers of 1 to 3. When b1 is greater than or equal to 2, two or more R₁'s may be the same or different from each other. Descriptions for b2 to b4, b11, b12 and b17 to b20 refer to description for b1 and structures of Formula 1 and Formulae 2A to 2F.

According to another embodiment; the condensed cyclic compound represented by the Formula 1 may be represented by one of the following Formulae 1-1 to 1-9:

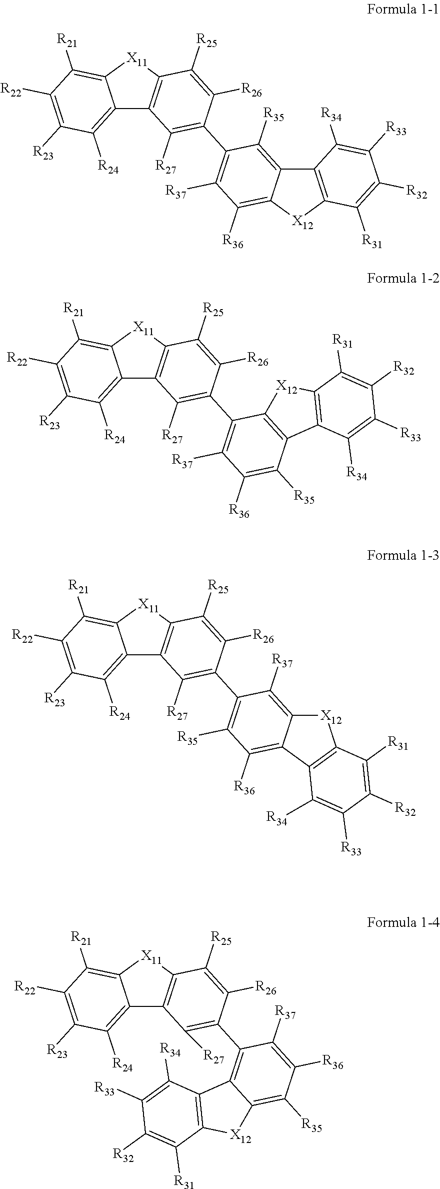

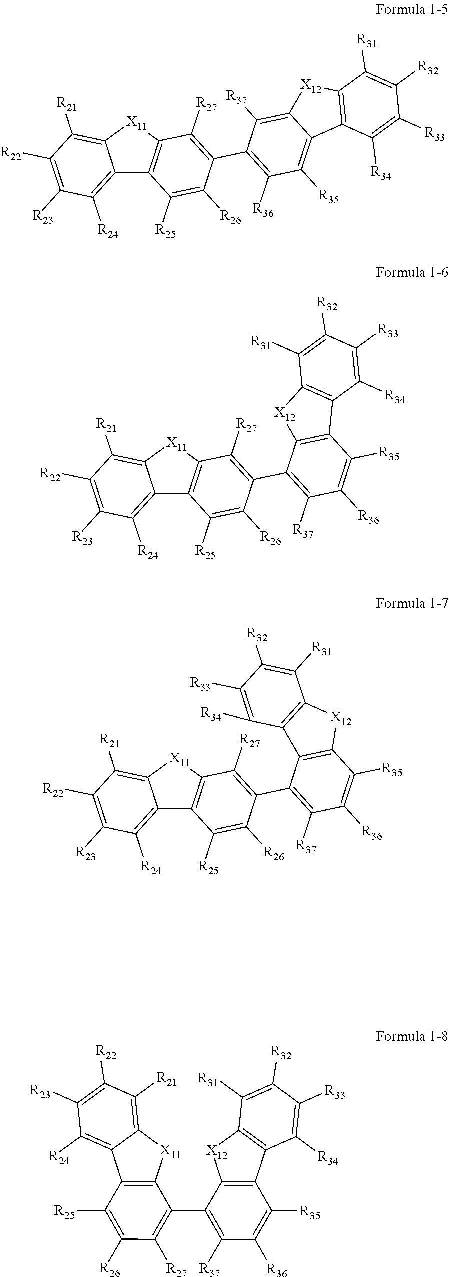

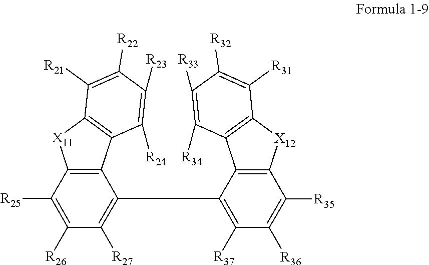

In Formulae 1-1 to 1-9, descriptions for X₁₁ and X₁₂ are the same as described in Formula 1, descriptions for R₂₁ to R₂₄ are the same as R₁₇, descriptions for R₂₅ to R₂₇ are the same as R₁₈, descriptions for R₃₁ to R₃₄ are the same as R₂₀, and descriptions for R₃₅ to R₃₇ are the same as R₁₉.

For example, in Formulae 1-1 to 1-9,

X₁₁ is selected from N-[(L₁₁)ₐ₁₁-(R₁₁)_{b11}], S, and O;

X₁₂ is selected from N-[(L₁₂)ₐ₁₂-(R₁₂)_{b12}], S, and O;

X₁ is selected from N-[(L₁)ₐ₁-(R₁)_{b1}], S, and O;

i) in Formulae 1-1 to 1-9, when X₁₁ is N-[(L₁₁)ₐ₁₁-(R₁₁)_{b11}], X₁₂ is not N-[(L₁₂)ₐ₁₂-(R₁₂)_{b12}], in Formula 1, at least one of b11 R₁₁'s and R₂₁ to R₂₇ and R₃₁ to R₃₇ is selected from the group represented by the Formula 2A to the group represented by the Formula 2F;

ii) in Formulae 1-1 to 1-9, when X₁₁ is not N-[(L₁₁)ₐ₁₁-(R₁₁)_{b11}], and X₁₂ is N-[(L₁₂)ₐ₁₂-(R₁₂)_{b12}], in Formula 1, at least one of b₁₂'s R₁₂ and R₂₁ to R₂₇ and R₃₁ to R₃₇ is selected from the group represented by the Formula 2A to the group represented by the Formula 2F; and

iii) in Formulae 1-1 to 1-9, when X₁₁ is N-[(L₁₁)ₐ₁₁-(R₁₁)_{b11}], and X₁₂ is N-[(L₁₂)ₐ₁₂-(R₁₂)_{b12}], in Formula 1, at least one of b11 R₁₁'s, and b12 R₁₂'s and R₂₁ to R₂₇ and R₃₁ to R₃₇ is selected from the group represented by the Formula 2A to the group represented by the Formula 2F;

the L₁ to L₄, L₁₁ and L₁₂ are independently selected from Formulae 2-1 to 2-13 (for example, Formulae 3-1 to 3-12);

the a1 to a4, a11 and a12 are independently 0 or 1;

the R₁, R₁₁ and R₁₂ are independently selected from

a phenyl group, a naphthyl group, a triphenylenyl group, a phenanthrenyl group, a pyridinyl group, a pyrimidinyl group, a pyrazinyl group, a pyridazinyl group, or a triazinyl group; and

a phenyl group, a naphthyl group, a triphenylenyl group, a phenanthrenyl group, a pyridinyl group, a pyrimidinyl group, a pyrazinyl group, a pyridazinyl group, and a triazinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a naphthyl group, a triphenylenyl group, a phenanthrenyl group, a pyridinyl group, a pyrimidinyl group, a pyrazinyl group, a pyridazinyl group, and a triazinyl group; and

the group represented by the Formula 2A to the group represented by the Formula 2F (provided that R₁ is not the group represented by the Formula 2A to the group represented by the Formula 2F);

at least one of the R₂ and R₃ is

a phenyl group, a naphthyl group, a triphenylenyl group, a phenanthrenyl group, a pyridinyl group, a pyrimidinyl group, a pyrazinyl group, a pyridazinyl group, or a triazinyl group; and

a phenyl group, a naphthyl group, a triphenylenyl group, a phenanthrenyl group, a pyridinyl group, a pyrimidinyl group, a pyrazinyl group, a pyridazinyl group, or a triazinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a naphthyl group, a triphenylenyl group, a phenanthrenyl group, a pyridinyl group, a pyrimidinyl group, a pyrazinyl group, a pyridazinyl group, and a triazinyl group;

the R₄ is selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and C₁-C₂₀ alkoxy group;

R₂₁ to R₂₇ and R₃₁ to R₃₇ are independently selected from

hydrogen, deuterium, —F, —Cl, —Br, —I, a hydroxyl group, a cyano group, a nitro group, an amino group, an amidino group, a hydrazine group, a hydrazine group, a carboxylic acid of a salt thereof, a sulfonic acid or a salt thereof, a phosphoric acid or a salt thereof, a C₁-C₂₀ alkyl group, and a alkoxy group;

a C₁-C₂₀ alkyl group and a C₁-C₂₀ alkoxy group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, and a phosphoric acid or a salt thereof; and

the Formulae 4-1 to 4-31 (for example, one of Formulae 5-1 to 5-63);

—Si(Q₃)(Q₄)(Q₅); and

the group represented by the Formula 2A to the group represented by the Formula 2F (provided that the R₁ to R₆ are not the group represented by the Formula 2A to the group represented by the Formula 2F), but are not limited thereto.

According to one embodiment, the condensed cyclic compound represented by the Formula 1 may be represented by the Formula 1-1, 1-3 or 1-5,

X₁₁ in Formula 1-1, 1-3 and 1-5 is N-[(L₁₁)ₐ₁₁-(R₁₁)_{b11}] and X₁₂ is N-[(L₁₂)ₐ₁₂-(R₁₂)_{b12}],

wherein the R₁₁ and R₁₂ are independently selected from

a phenyl group, a naphthyl group, a triphenylenyl group, a phenanthrenyl group, a pyridinyl group, a pyrimidinyl group, a pyrazinyl group, a pyridazinyl group, of a triazinyl group; and

a phenyl group, a naphthyl group, a triphenylenyl group, a phenanthrenyl group, a pyridinyl group, a pyrimidinyl group, a pyrazinyl group, a pyridazinyl group, or a triazinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a naphthyl group, a triphenylenyl group, a phenanthrenyl group, a pyridinyl group, a pyrimidinyl group, a pyrazinyl group, a pyridazinyl group, and a triazinyl group; and

the group represented by the Formula 2A to the group represented by the Formula 2F; and

at least one of the R₁₁ and R₁₂ may be selected from the group represented by the Formula 2A to the group represented by the Formula 2F, but is not limited thereto.

The condensed cyclic compound represented by the Formula 1 may be one of the following compounds 1 to 30, but is not limited thereto.

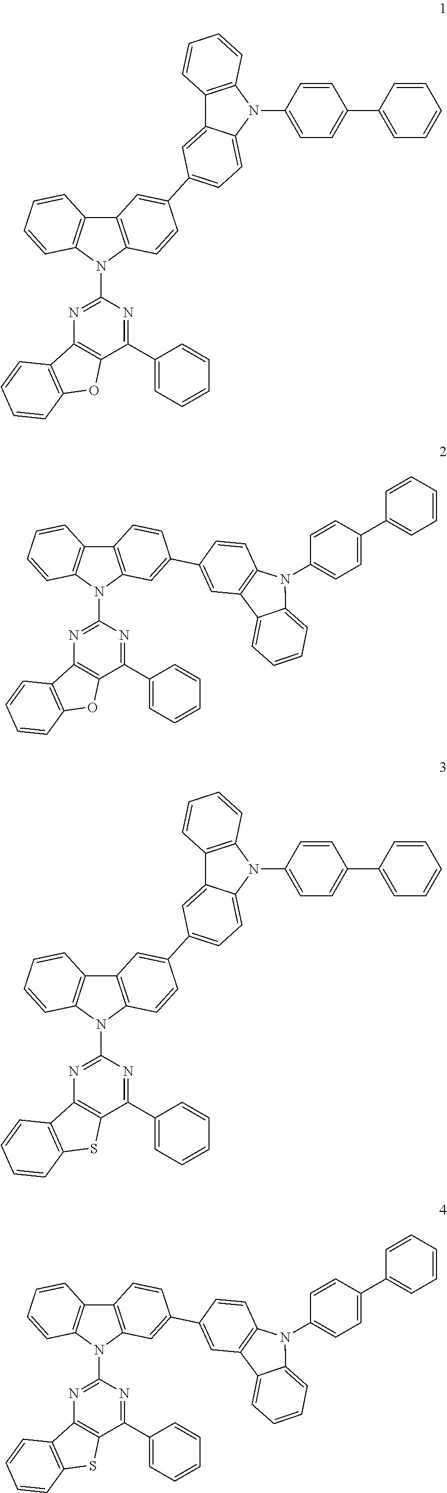 [SMILES: c1ccc(-c2ccc(-n3c4ccccc4c4cc(-c5ccc6c(c5)c5ccccc5n6-c5nc(-c6ccccc6)c6oc7ccccc7c6n5)ccc43)cc2)cc1 ; c1ccc(-c2ccc(-n3c4ccccc4c4cc(-c5ccc6c7ccccc7n(-c7nc(-c8ccccc8)c8oc9ccccc9c8n7)c6c5)ccc43)cc2)cc1 ; c1ccc(-c2ccc(-n3c4ccccc4c4cc(-c5ccc6c(c5)c5ccccc5n6-c5nc(-c6ccccc6)c6sc7ccccc7c6n5)ccc43)cc2)cc1 ; c1ccc(-c2ccc(-n3c4ccccc4c4cc(-c5ccc6c7ccccc7n(-c7nc(-c8ccccc8)c8sc9ccccc9c8n7)c6c5)ccc43)cc2)cc1]

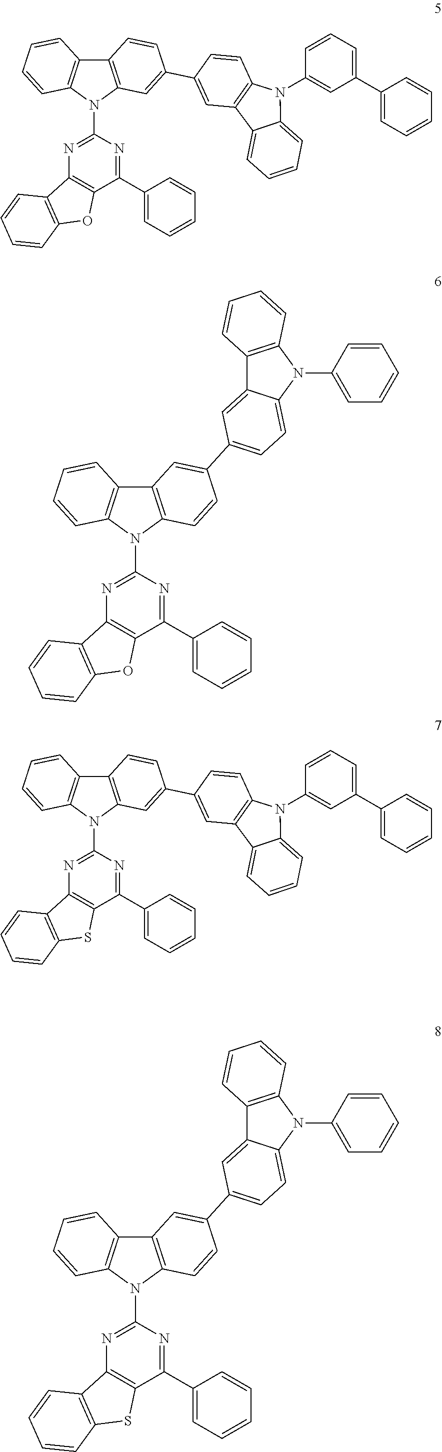 [SMILES: c1ccc(-c2cccc(-n3c4ccccc4c4cc(-c5ccc6c7ccccc7n(-c7nc(-c8ccccc8)c8oc9ccccc9c8n7)c6c5)ccc43)c2)cc1 ; c1ccc(-c2nc(-n3c4ccccc4c4cc(-c5ccc6c(c5)c5ccccc5n6-c5ccccc5)ccc43)nc3c2oc2ccccc23)cc1 ; c1ccc(-c2cccc(-n3c4ccccc4c4cc(-c5ccc6c7ccccc7n(-c7nc(-c8ccccc8)c8sc9ccccc9c8n7)c6c5)ccc43)c2)cc1 ; c1ccc(-c2nc(-n3c4ccccc4c4cc(-c5ccc6c(c5)c5ccccc5n6-c5ccccc5)ccc43)nc3c2sc2ccccc23)cc1]

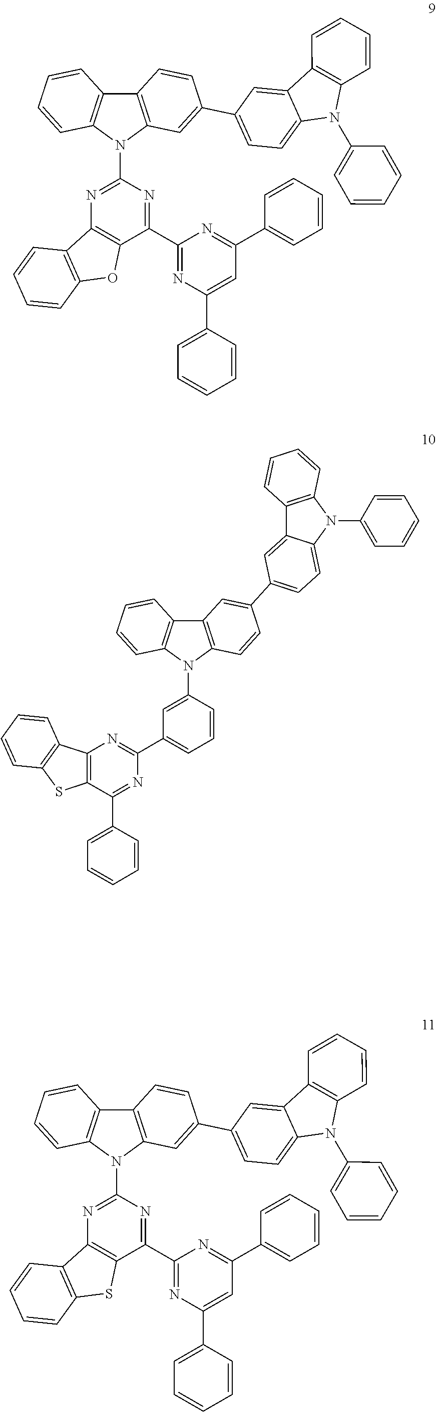 [SMILES: c1ccc(-c2cc(-c3ccccc3)nc(-c3nc(-n4c5ccccc5c5ccc(-c6ccc7c(c6)c6ccccc6n7-c6ccccc6)cc54)nc4c3oc3ccccc34)n2)cc1 ; c1ccc(-c2nc(-c3cccc(-n4c5ccccc5c5cc(-c6ccc7c(c6)c6ccccc6n7-c6ccccc6)ccc54)c3)nc3c2sc2ccccc23)cc1 ; c1ccc(-c2cc(-c3ccccc3)nc(-c3nc(-n4c5ccccc5c5ccc(-c6ccc7c(c6)c6ccccc6n7-c6ccccc6)cc54)nc4c3sc3ccccc34)n2)cc1]

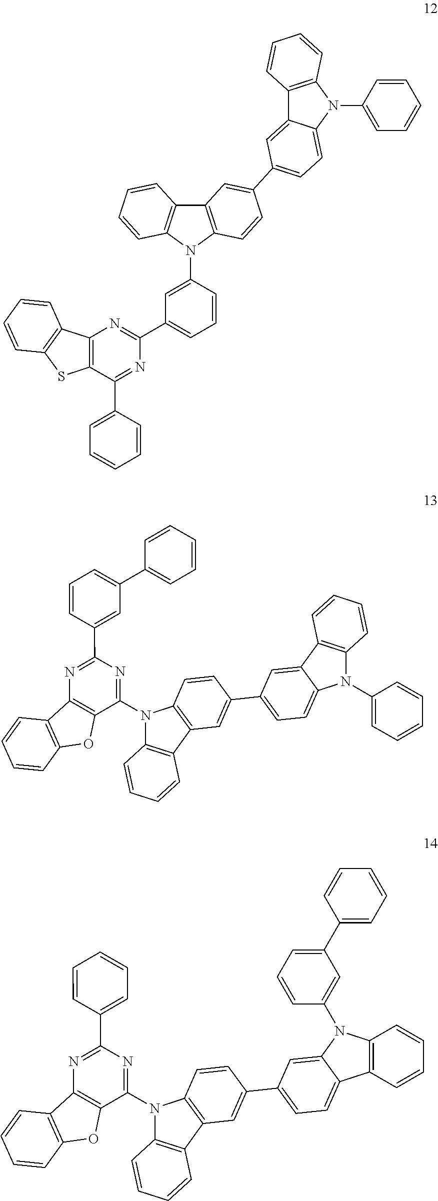 [SMILES: c1ccc(-c2nc(-c3cccc(-n4c5ccccc5c5cc(-c6ccc7c(c6)c6ccccc6n7-c6ccccc6)ccc54)c3)nc3c2sc2ccccc23)cc1 ; c1ccc(-c2cccc(-c3nc(-n4c5ccccc5c5cc(-c6ccc7c(c6)c6ccccc6n7-c6ccccc6)ccc54)c4oc5ccccc5c4n3)c2)cc1 ; c1ccc(-c2cccc(-n3c4ccccc4c4ccc(-c5ccc6c(c5)c5ccccc5n6-c5nc(-c6ccccc6)nc6c5oc5ccccc56)cc43)c2)cc1]

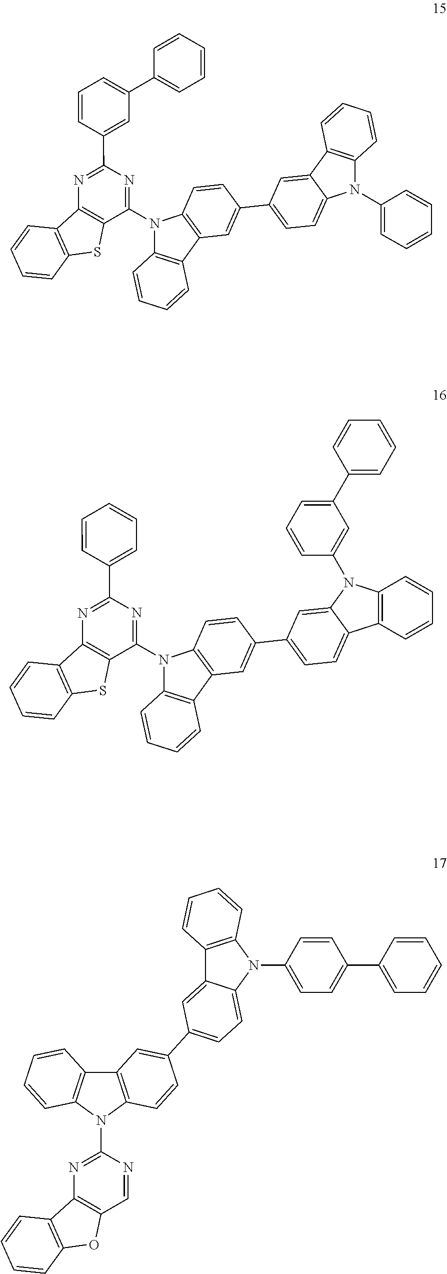 [SMILES: c1ccc(-c2cccc(-c3nc(-n4c5ccccc5c5cc(-c6ccc7c(c6)c6ccccc6n7-c6ccccc6)ccc54)c4sc5ccccc5c4n3)c2)cc1 ; c1ccc(-c2cccc(-n3c4ccccc4c4ccc(-c5ccc6c(c5)c5ccccc5n6-c5nc(-c6ccccc6)nc6c5sc5ccccc56)cc43)c2)cc1 ; c1ccc(-c2ccc(-n3c4ccccc4c4cc(-c5ccc6c(c5)c5ccccc5n6-c5ncc6oc7ccccc7c6n5)ccc43)cc2)cc1]

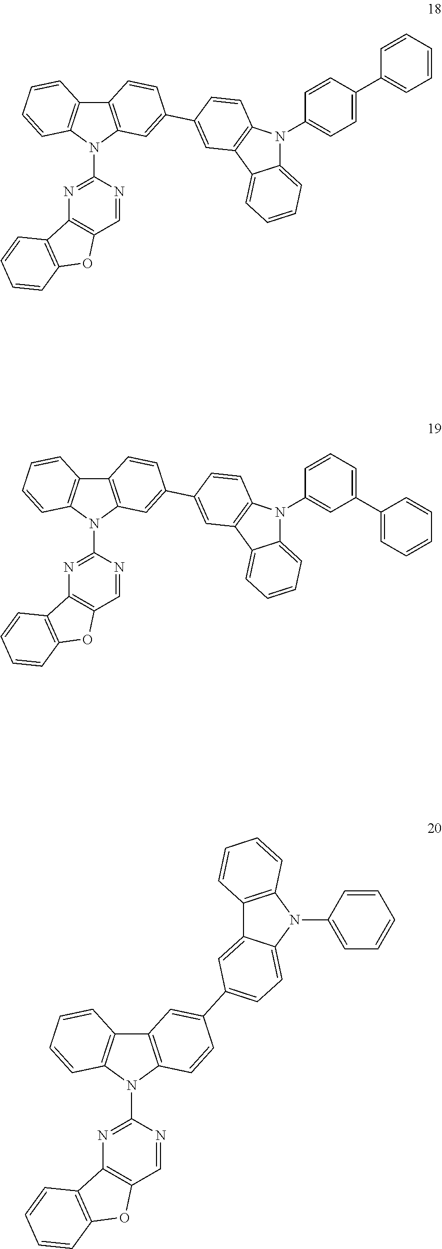 [SMILES: c1ccc(-c2ccc(-n3c4ccccc4c4cc(-c5ccc6c7ccccc7n(-c7ncc8oc9ccccc9c8n7)c6c5)ccc43)cc2)cc1 ; c1ccc(-c2cccc(-n3c4ccccc4c4cc(-c5ccc6c7ccccc7n(-c7ncc8oc9ccccc9c8n7)c6c5)ccc43)c2)cc1 ; c1ccc(-n2c3ccccc3c3cc(-c4ccc5c(c4)c4ccccc4n5-c4ncc5oc6ccccc6c5n4)ccc32)cc1]

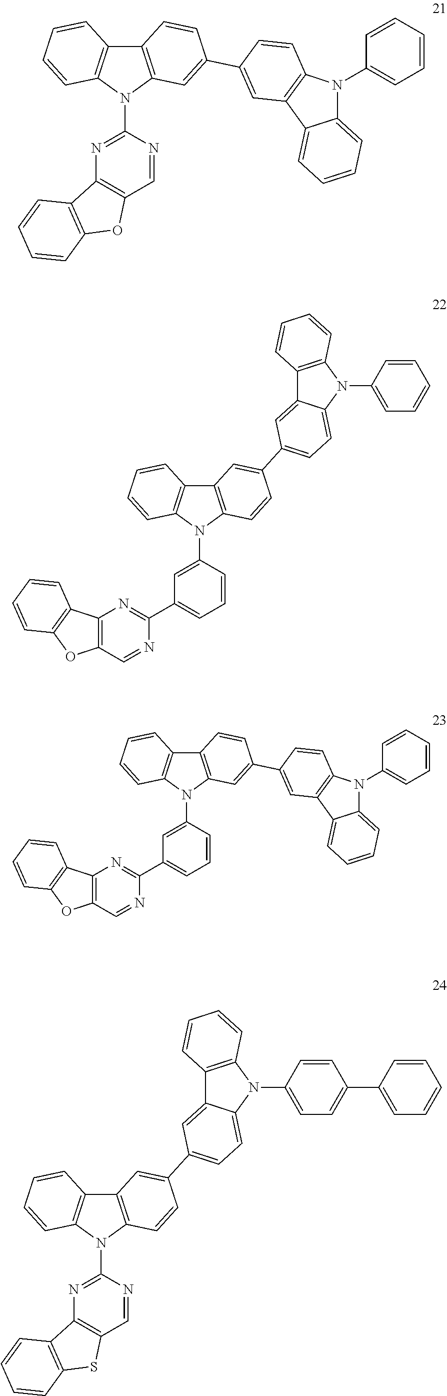 [SMILES: c1ccc(-n2c3ccccc3c3cc(-c4ccc5c6ccccc6n(-c6ncc7oc8ccccc8c7n6)c5c4)ccc32)cc1 ; c1ccc(-n2c3ccccc3c3cc(-c4ccc5c(c4)c4ccccc4n5-c4cccc(-c5ncc6oc7ccccc7c6n5)c4)ccc32)cc1 ; c1ccc(-n2c3ccccc3c3cc(-c4ccc5c6ccccc6n(-c6cccc(-c7ncc8oc9ccccc9c8n7)c6)c5c4)ccc32)cc1 ; c1ccc(-c2ccc(-n3c4ccccc4c4cc(-c5ccc6c(c5)c5ccccc5n6-c5ncc6sc7ccccc7c6n5)ccc43)cc2)cc1]

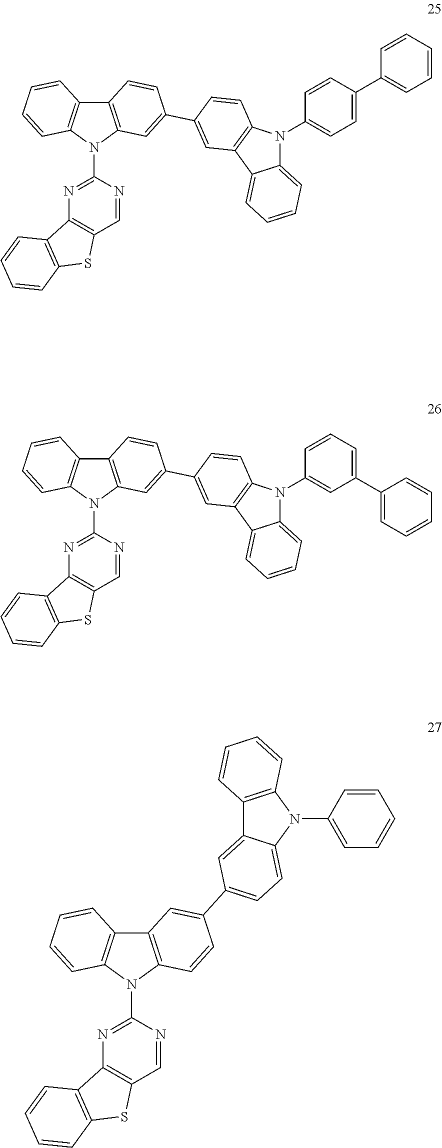 [SMILES: c1ccc(-c2ccc(-n3c4ccccc4c4cc(-c5ccc6c7ccccc7n(-c7ncc8sc9ccccc9c8n7)c6c5)ccc43)cc2)cc1 ; c1ccc(-c2cccc(-n3c4ccccc4c4cc(-c5ccc6c7ccccc7n(-c7ncc8sc9ccccc9c8n7)c6c5)ccc43)c2)cc1 ; c1ccc(-n2c3ccccc3c3cc(-c4ccc5c(c4)c4ccccc4n5-c4ncc5sc6ccccc6c5n4)ccc32)cc1]

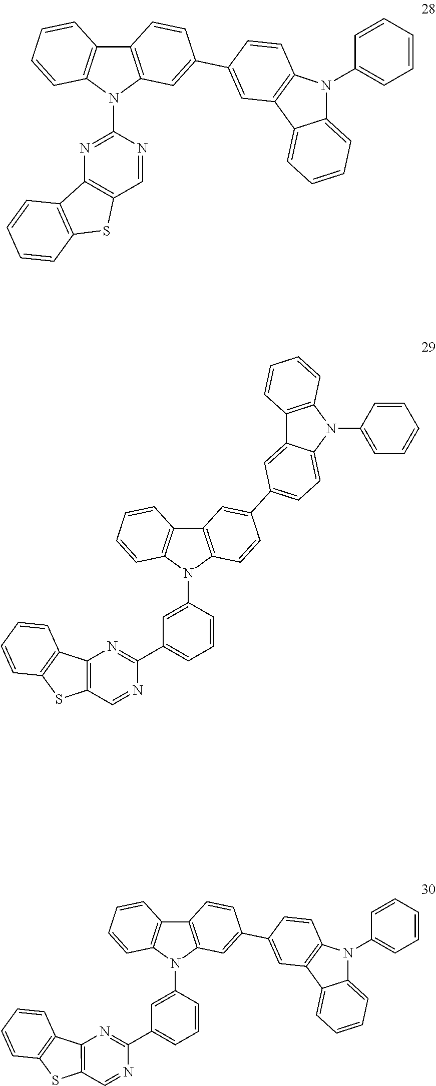 [SMILES: c1ccc(-n2c3ccccc3c3cc(-c4ccc5c6ccccc6n(-c6ncc7sc8ccccc8c7n6)c5c4)ccc32)cc1 ; c1ccc(-n2c3ccccc3c3cc(-c4ccc5c(c4)c4ccccc4n5-c4cccc(-c5ncc6sc7ccccc7c6n5)c4)ccc32)cc1 ; c1ccc(-n2c3ccccc3c3cc(-c4ccc5c6ccccc6n(-c6cccc(-c7ncc8sc9ccccc9c8n7)c6)c5c4)ccc32)cc1]

The condensed cyclic compound represented by the Formula 1 necessarily includes at least one of the groups represented by formulae 2A to 2F. Thereby, the condensed cyclic compound represented by the Formula 1 has bipolar characteristics, and thus may have HOMO, LUMO, an energy gap, and a T1 energy level that are appropriate used for a material for an organic light emitting device (for example, a host of an emission layer).

A method of synthesizing the condensed cyclic compound represented by the Formula 1 may be easily understood by a person of an ordinary skill in the art with a reference to the post-described Synthesis Examples.

Accordingly, the condensed cyclic compound represented by the Formula 1 may be appropriately applied to an organic layer for an organic light emitting device, for example, a host in an emission layer as the organic layer, and another embodiment of the present invention provides an organic light emitting device including a first electrode, a second electrode; and the organic layer interposed between the first electrode and the second electrode, including an emission layer, and including at feast one condensed cyclic compound represented by the Formula 1.

The organic light emitting device includes the organic layer including the condensed cyclic compound represented by the Formula 1 and thus, may have a low driving voltage, high efficiency, high luminance, and a long life-span.

The condensed cyclic compound represented by the Formula 1 may be used between a pair of electrodes of an organic light emitting device. For example, the condensed cyclic compound may be included in a hole transport region (for example, at least one of a hole injection layer (HIL), a hole transport layer (HTL) and an electron blocking layer) between the emission layer and the first electrode and in an electron transport region (for example, at least one of a hole blocking layer, an electron transport layer (ETL) and an electron injection layer (EIL) between the emission layer and the second electrode. For example, the condensed cyclic compound represented by the Formula 1 may be included in the emission layer. Herein, the emission layer may further include a dopant, and the condensed cyclic compound included in the emission layer may play a role of a host. The emission layer may be a blue emission layer emitting blue light or a green emission layer emitting green light, and the dopant may be a phosphorescent dopant.

In the present specification, “(an organic layer) includes at least one condensed cyclic compound” may be understood as “(the organic layer) may include one kind of condensed cyclic compound belonging to the Formula 1 or more than two condensed cyclic compounds belonging to the Formula 1.”

For example, the organic layer may include only the compound 1 as the condensed cyclic compound. Herein, the compound 1 may be present in the emission layer of an organic light emitting device. In addition, the organic layer may include the compounds 1 and 2 as the condensed cyclic compound. Herein, the compounds 1 and 2 may be present in the same layer (for example, the compounds 1 and 2 may be present in the emission layer) or in different layers.

The first electrode is an anode of a hole injection electrode, while the second electrode is a cathode of an electron injection electrode, or the first electrode is a cathode of an electron injection electrode, while the second electrode is a cathode of a hole injection electrode.

For example, the first electrode is an anode, the second electrode is a cathode, and the organic layer may include i) a hole transport region interposed between the first electrode and the emission layer and including at least one of a hole injection layer (HIL), a hole transport layer (HTL) and electron blocking layer; and ii) an electron transport region interposed between the emission layer and the second electrode and including at least one of a hole blocking layer, an electron transport layer (ETL) and an electron injection layer (EIL).

In the present specification, the “organic layer” is a term indicating a single layer and/or a plurality of layers interposed between first and second electrodes in an organic light emitting device. The “organic layer” may include an organic metal complex including a metal and the like as well as an organic compound.

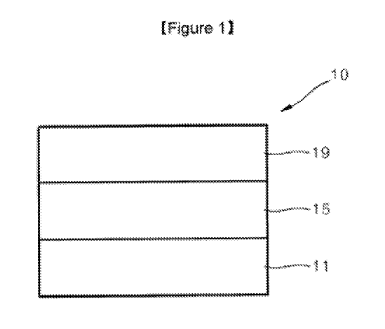

FIG. 1 is a schematic cross-sectional view of an organic light emitting device 10 according to one embodiment of the present invention. Hereinafter, referring to FIG. 1, a structure and a manufacturing method an organic light emitting device according to one embodiment of the present invention are described as follows. An organic light emitting device 10 has a structure where a first electrode 11, an organic layer 15 and a second electrode 19 are sequentially stacked.

A substrate may be additionally disposed beneath the first electrode 11 or on the second electrode 13. The substrate may be any substrate used in an organic light emitting device and for example, include a glass substrate or a transparent plastic substrate having excellent mechanical strength, thermal stability, transparency, surface flatness, easy handling and waterproof.

The first electrode 11 may be fabricated by depositing, for example, a material for the first electrode on the substrate in a deposition method, a sputtering method or the like. The first electrode 11 may be an anode. The material for the first electrode may be selected from the materials having a high work function to facilitate a hole injection. The first electrode 11 may be a reflective electrode, a transflective electrode or a transmissive electrode. The material for the first electrode may be indium tin oxide (ITO), indiumzinc oxide (IZO), tin oxide (SnO₂), zinc oxide (ZnO) and the like. In addition, a metal such as magnesium (Mg), aluminum (Al), aluminum-lithium (Al—Li), calcium (Ca), magnesium-indium (Mg—In), magnesium-silver (Mg—Ag) and the like may be used.

The first electrode 11 may have a monolayer structure or a multi-layer structure including more than two layers.

On the first electrode 11, an organic layer 15 is disposed.

The organic layer 15 may include a hole transport region; an emission layer; and an electron transport region.

The hole transport region may be disposed between the first electrode 11 and the emission layer.

The hole transport region may include at least one of a hole injection layer (HIL), a hole transport layer (HTL), an electron blocking layer and a buffer layer.

The hole transport region includes only the hole injection layer (HIL) or the hole transport layer (HTL). Or, the hole transport region may have a structure of hole injection layer (HIL)/hole transport layer (HTL) or hole injection layer (HIL)/hole transport layer (HTL)/electron blocking layer sequentially stacked from the first electrode 11.

When the hole transport region includes the hole injection layer (HIL), the hole injection layer (HIL) may be formed on the first electrode 11 in a vacuum deposition method, a spin coating method, a cast method, a LB method and the like.

When the vacuum deposition method is used to form the hole injection layer (HIL), the deposition may be performed under various conditions depending on a compound as a material for the hole injection layer (HIL), its structure and thermal characteristics and the like of the hole injection layer (HIL) but, for example, at a temperature of about 100 to about 500° C., a vacuum degree of about 10⁻⁸ to about 10⁻³ torr, at a deposit speed of about 0.01 to about 100 Å/sec, without limitation.

The spin coating method of forming the hole injection layer (HIL) may be performed under various conditions depending on a compound as a material for the hole injection layer (HIL), its structure and thermal characteristics and the like of the hole injection layer (HIL) but, for example, at a coating speed of about 2000 rpm to about 5000 rpm and a heat treatment temperature of about 80° C. to 200° C. for removing a solvent after the coating, without limitation.

The hole transport layer (HTL) and the electron blocking layer are formed referring to the conditions for forming the hole injection layer (HIL).

The hole transport region may include, for example, at least one of m-MTDATA, TDATA, 2-TNATA, NPB, β-NPB, TPD, Spiro-TPD, Spiro-NPB, α-NPB, TAPC, HMTPD, TCTA (4,4′,4″-tris(N-carbazolyl)triphenylamine), Pani/DBSA (polyaniline/dodecylbenzenesulfonic acid), PEDOT/PSS (poly (3,4-ethylenedioxythiophene)/poly(4-styrenesulfonate)), Pani/CSA (polyanilene/camphor sulfonic acid), PANI/PSS (polyaniline)/poly(4-styrenesulfonate)), a compound represented by the following Formula 201 and a compound represented by the following Formula 202:

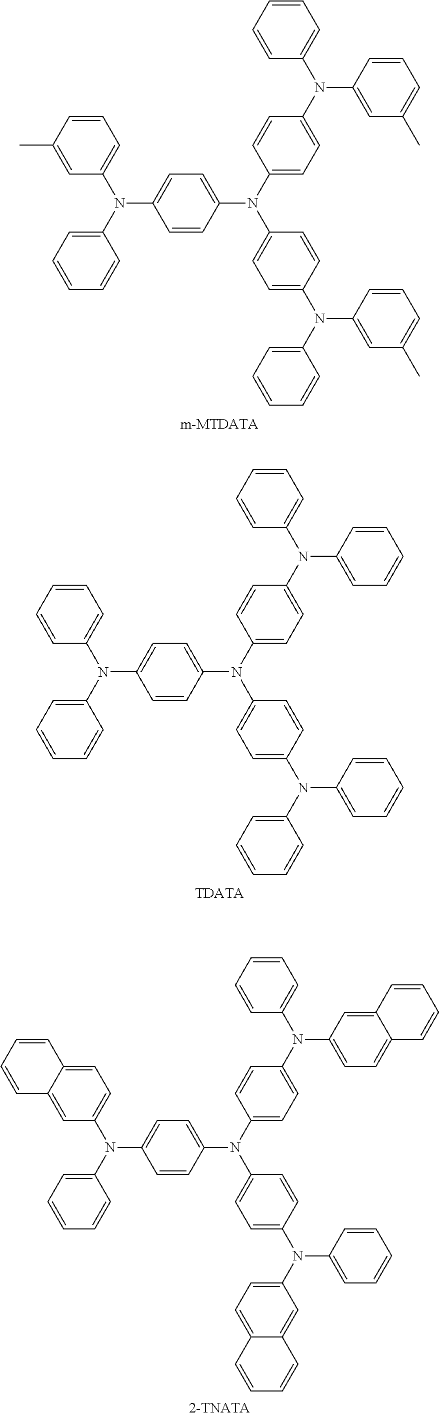

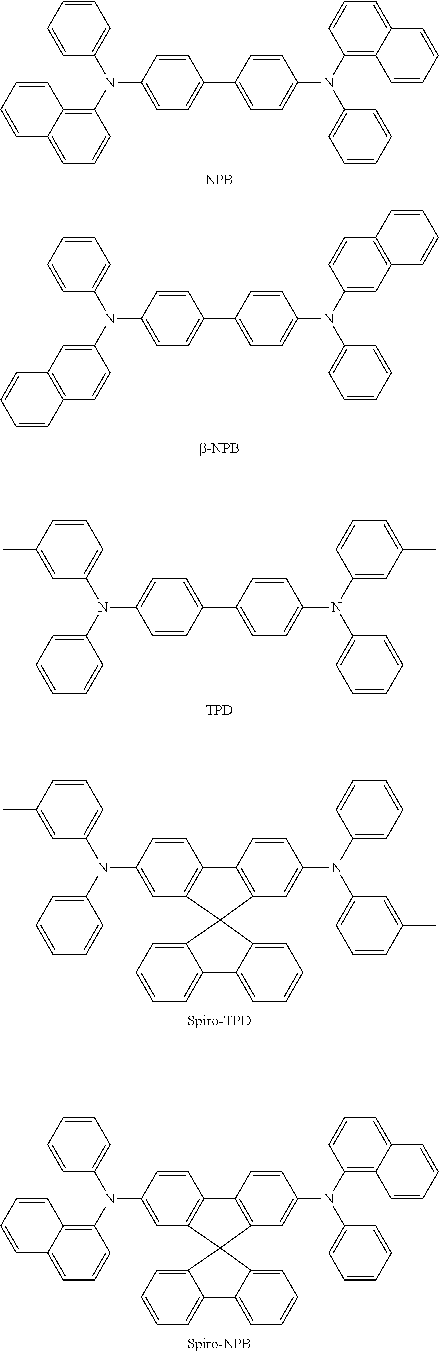

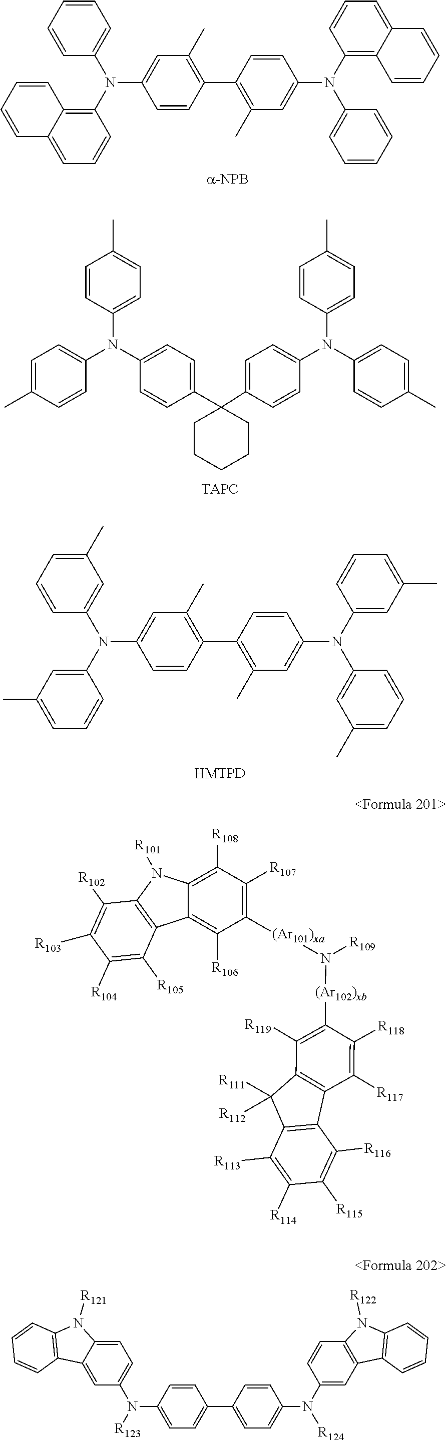

In Formula 201, Ar₁₀₁ and Ar₁₀₂ are independently

a phenylene; group, a pentalenylene groups an indenylene group, a naphthylene group, an azulenylene group, a heptalenylene group, an acenaphthylene group, a fluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, and a pentacenylene group; and

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group or a pentacenylene group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₂-C₁₀ heterocycloalkyl group, a C₂-C₁₀ heterocycloalkenyl group, a C₆-C₆₀ aryl group, a C₆-C₆₀ aryloxy group, a C₆-C₆₀ arylthio group, a C₂-C₆₀heteroaryl group, a monovalent non-aromatic condensed polycyclic group, and a monovalent non-aromatic heterocondensed polycyclic group.

In Formula 201, the xa and xb may be independently integers of 0 to 5, or 0, 1 or 2. For example, the xa may be 1, xa and xb may be 0, but are not limited thereto.

In Formulae 201 and 202, the R₁₀₁ to R₁₀₈, R₁₁₁ to R₁₁₉ and R₁₂₁ to R₁₂₄ may be independently selected from

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, a pentyl group, a hexyl group, and the like) and a alkoxy group (for example, a methoxy group, an ethoxy group, a propoxy group, a butoxy group, a pentoxy group, and the like);

a C₁-C₁₀ alkyl group and a C₁-C₁₀ alkoxy group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group and a pyrenyl group; and

a phenyl group, a naphthyl group, an anthracenyl group, a fluorenyl group or a pyrenyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group, but are not limited thereto.

In Formula 201, R₁₀₉ is one of a phenyl group, a naphthyl group, an anthracenyl group, and a pyridinyl group; and a phenyl group, a naphthyl group, an anthracenyl group or a pyridinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group.

According to one embodiment, the compound represented by the Formula 201 may be represented by the following Formula 201 A, but is not limited thereto:

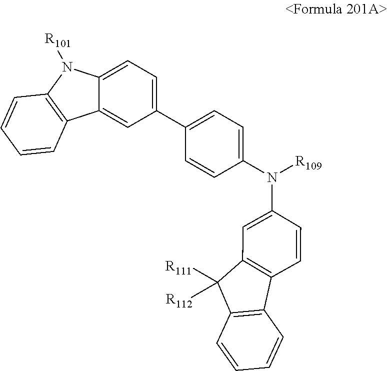

In Formula 201A, the decreiptions for R₁₀₁, R₁₁₁, R₁₁₂ and R₁₀₉ refer to the detailed description.

For example, the compound represented by the Formula 201 and the compound represented by the Formula 202 may include the following compounds HT1 to HT20, but are not limited thereto:

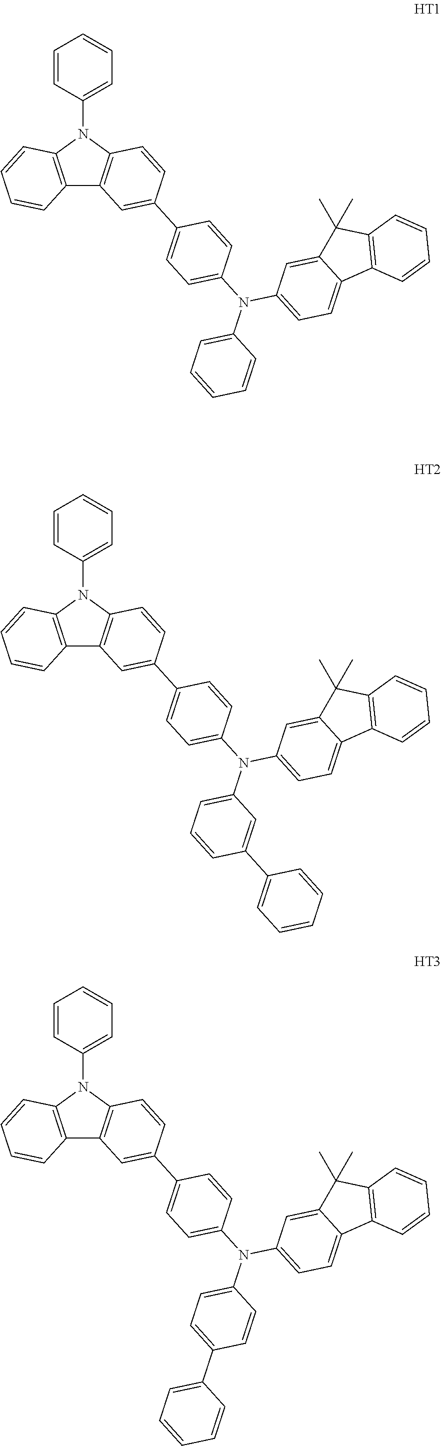 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

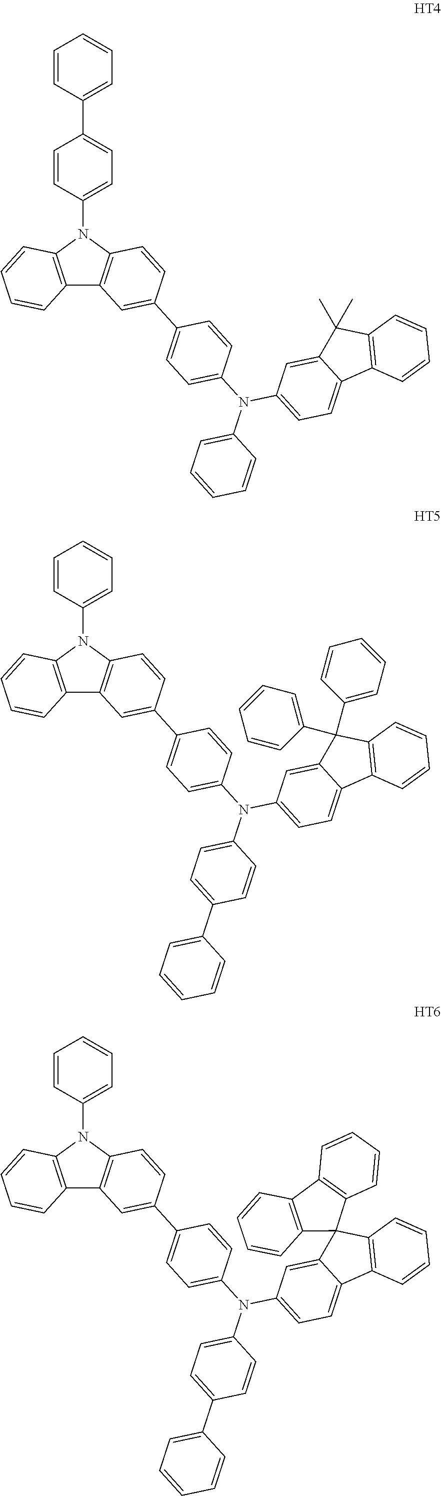 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

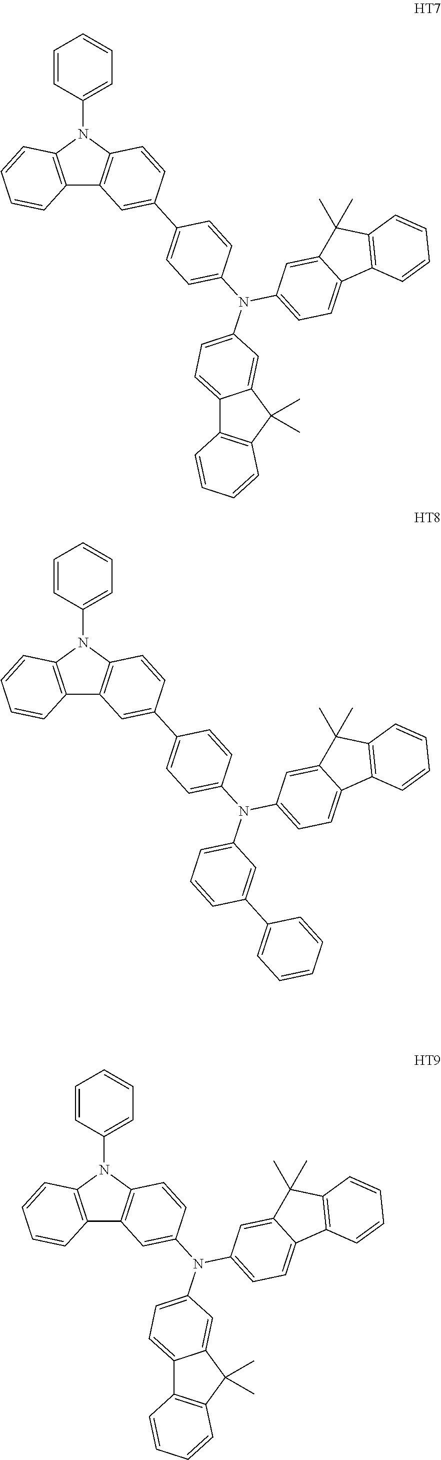 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

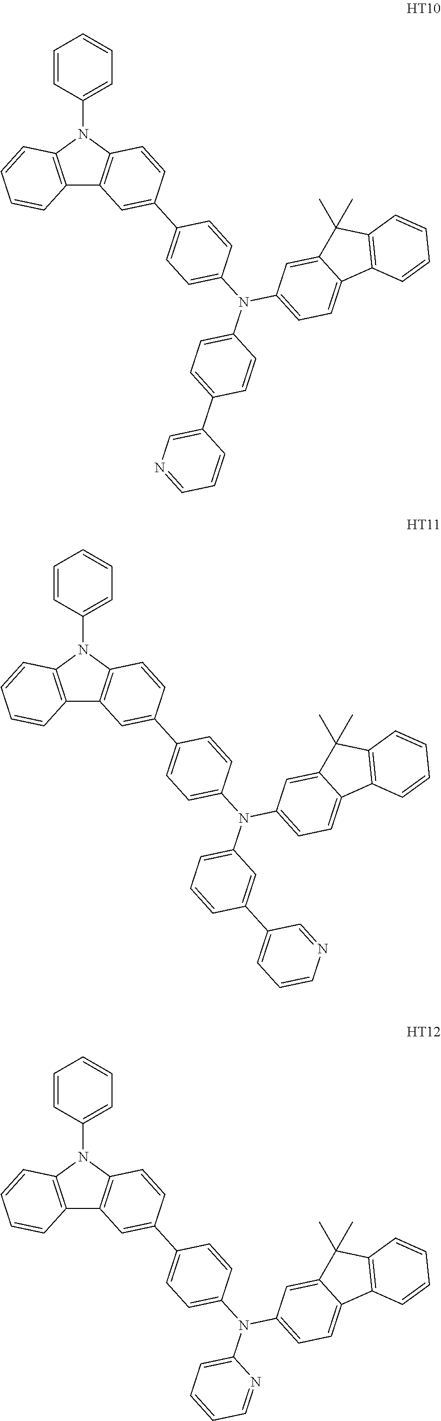 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

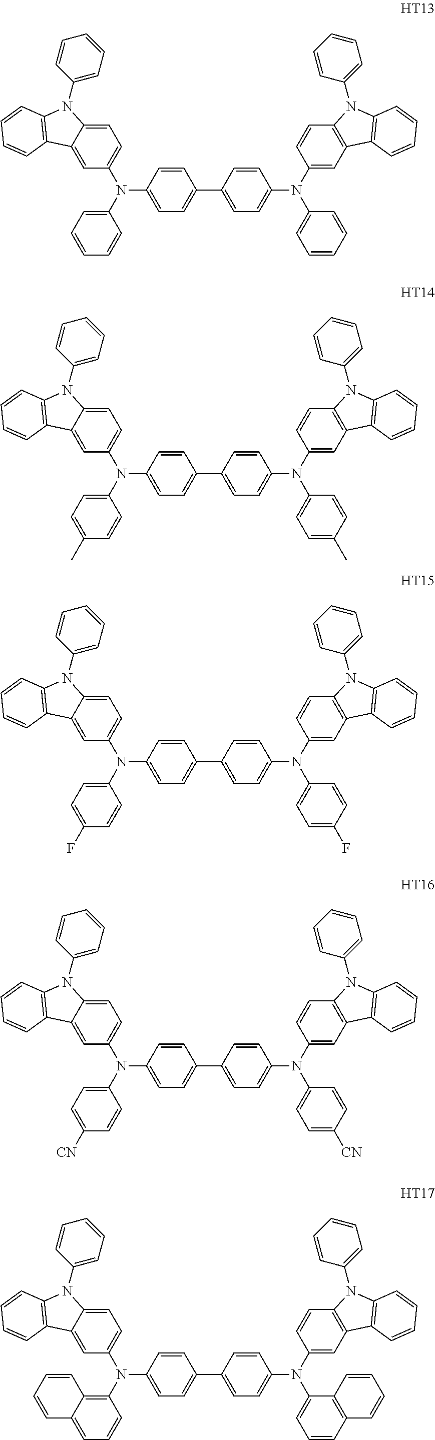

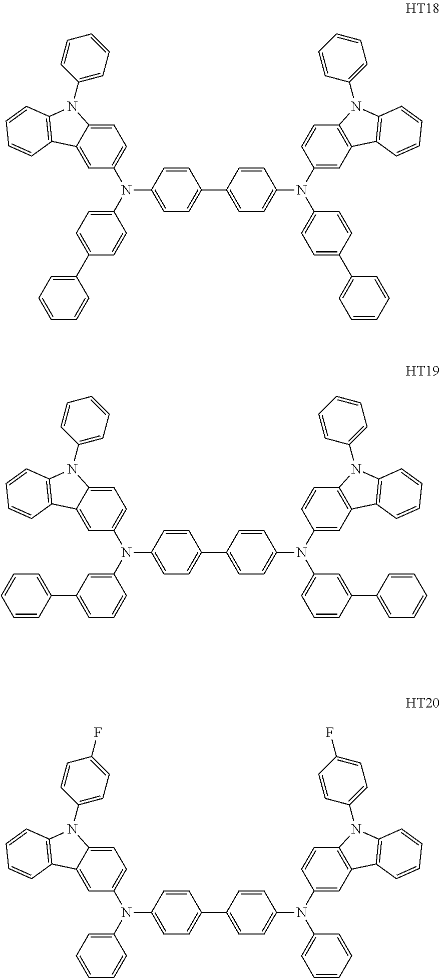 [SMILES: c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

The hose transport region may be about 100 Å to about 10000 Å thick, for example, about 100 Å to about 1000 Å thick. When the bole transport region includes the hole injection layer (HIL) and the hole transport layer (HIL), the hole injection layer (HIL) may be about 100 Å to about 10000 Å thick, for example, about 100 Å to about 1000 Å thick, while the hole transport layer (HTL) may be about 50 Å to about 2000 Å thick, for example, about 100 Å to about 1500 Å thick. When the hole transport region, the hole injection layer (HIL) and the hole transport layer (HTL) have a thickness within the ranges, satisfactory hole transport characteristics may be obtained without substantially increasing a driving voltage.

The hole transport region may further include a charge-producing material other than the material to improve conductivity. The charge-producing material may be uniformly or non-uniformly dispersed in the hole transport region.

The charge-producing material may be, for example, a p-dopant. The p-dopant may be one of a quinone derivative, a metal oxide and a cyano group-containing compound but is not limited thereto. For example, non-limiting examples of the p-dopant may be a quinone derivative such as tetracyanoquinonedimethane (TCNQ) and 2,3,5,6-tetrafluoro-tetracyano-1,4-benzoquinonedimethane (F4-TCNQ) and the like; a metal oxide such as tungsten oxide, molybdenum oxide and the like; and a cyano group-containing compound such as the following compound HT-D1 and the like, but are not limited thereto.

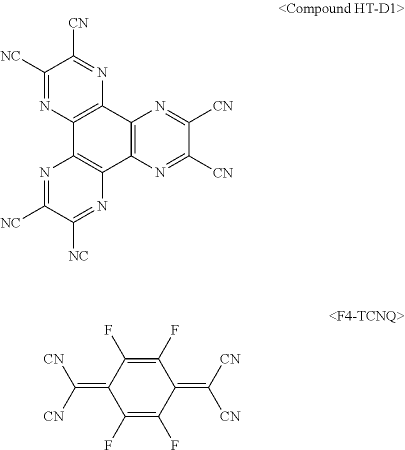

The hole transport region may further include a buffer layer.

The buffer layer may play a role of compensating an optical resonance distance depending on wavelength of light emitted from the emission layer and thus, increasing efficiency.

On the hole transport region, an emission layer (EML) may be formed in a vacuum deposition method, a spin coating method, a cast method, a LB method and the like. When the vacuum deposition method and the spin coating method are used to form the emission layer, their deposit and coating conditions may vary depending on a compound used therein and in general, may be selected out of almost the same condition range as that of the hole injection layer (HIL).

When the organic light emitting device is a full color organic light emitting device, the emission layer may be patterned in an order of a red emission layer, a green emission layer and a blue emission layer. Or, the emission layer has a structure of stacking the red emission layer, the green emission layer and/or the blue emission layer and thus, may be variously modified, for example, emits white light.

The emission layer may include a host and a dopant. The host may be a condensed cyclic compound represented by the Formula 1. According to one embodiment, the emission layer may be a green emission layer emitting green light.

The dopant in the emission layer is a fluorescent dopant emitting light according to a fluorescence emission mechanism or a phosphorescent dopant emitting light according to a phosphorescence emission mechanism.

According to another embodiment, the host includes a first host and a second host that are different from each other, wherein the first host includes the condensed cyclic compound represented by the Formula 1, the second host includes at least one of a first compound represented by the following Formula 41 and a second compound represented by the following Formula 61:

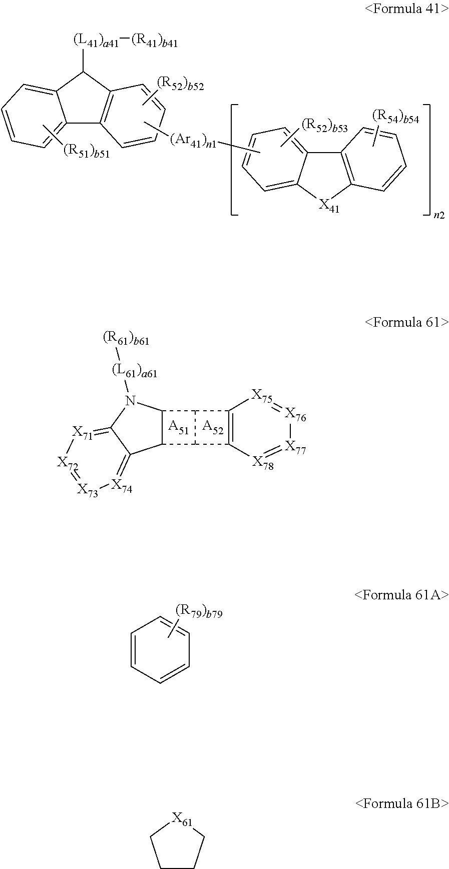

In Formulae 41, 61, 61A and 61B

X₄₁ selected from N-[(L₄₂)ₐ₄₂-(R₄₂)_{b42}], S, O, (S═O), S(═O)₂, C(═O), C(R₄₃)(R₄₄), Si(R₄₃)(R₄₄), P(R₄₃), P(═O)R₄₃, and C═N(R₄₃);

in Formula 61, the ring A₆₁ is represented by the Formula 61A;

in Formula 61, the ring A₆₂ is represented by the Formula 61B;

X₆₁ is selected from N-[(L₆₂)ₐ₆₂-(R₆₂)_{b62}], S, O, S(═O), S(═O)₂, C(═O), C(R₆₃)(R₆₄), Si(R₆₃)(R₆₄), P(R₆₃), P(═O)R₆₃, and C═N(R₆₃);

X₇₁ is C(R₇₁) or N, X₇₂ is C R₇₂ or N, X₇₃ is C(R₇₃) or N, X₇₄ is C(R₇₄) or N, X₇₅ is C(R₇₅) or N, X₇₆ is C(R₇₆) or N, X₇₇ is C(R₇₇) or N, X₇₆ is C(R₇₈) or N;

the descriptions for Ar₄₁, L₄₁, L₄₂, L₆₁ and L₆₂ refer to description for L₁;

n1 and n2 are independently selected from integers of 0 to 3;

a41, a42, a61 and a62 are independently selected from integers of 0 to 3;,

R₄₁ to R₄₃, R₅₁ to R₅₄, R₆₁ to R₆₄ and R₇₁ to R₇₉ are independently selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀heteroaryl group, a substituted or unsubstituted monovalent non-aromatic condensed polycyclic group, a substituted or unsubstituted monovalent non-aromatic heterocondensed polycyclic group, —N(Q₁)(Q₂), —Si(Q₃)(Q₄)Q₅), and —B(Q₆)(Q₇); and

b41, b42, b51 to b54, b61, b62 and b79 are independently selected from integers of 1 to 3.

According to one embodiment, the R₄₁ to R₄₃, R₅₁ to R₅₄, R₆₁ to R₆₄ and R₇₁ to R₇₉ are independently selected from

hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and a C₁-C₂₀ alkoxy group;

a C₁-C₂₀ alkyl group and a C₁-C₂₀ alkyl group, each of which is substituted with at least one of deuterium, —F, —Cl, —Br, —I, hydroxyl group, a cyano group, a nitro group, an amino group, an amidino group, a hydrazine group, a hydrazone group, a carboxylic acid or a salt thereof, a sulfonic acid or a salt thereof, and a phosphoric acid or a salt thereof;

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiopbenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group and an imidazopyrimidinyl group, each of which is substituted with one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, —Si(Q₃₃)(Q₃₄)(Q₃₅), a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, and a biphenyl group; and

—Si(Q₃)(Q₄)(Q₅) (provided that, the R₁₃ to R₁₆ and R₅ and R₆ are not —Si(Q₃)(Q₄)(Q₅));

wherein the Q₃ to Q₅ and Q₃₃ to Q₃₅ are independently selected from hydrogen, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a chrysenyl group, a Carbazolyl group, a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group, and a quinoxalinyl group, but are not limited thereto.

For example, the first compound may be represented by one of the following Formulae 41-1 to 41-12, and the second compound may be represented by one of the following Formulae 61-1 to 61-6:

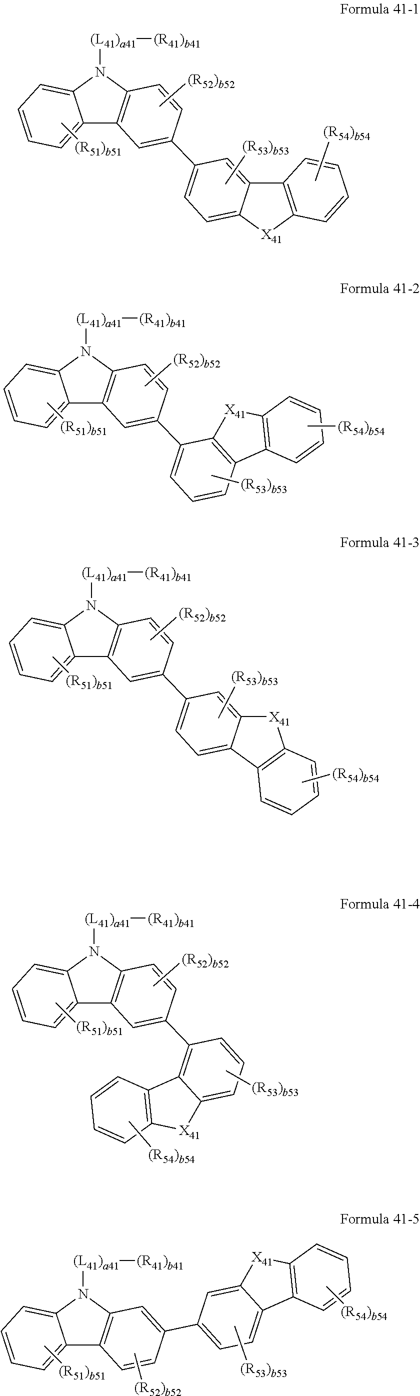

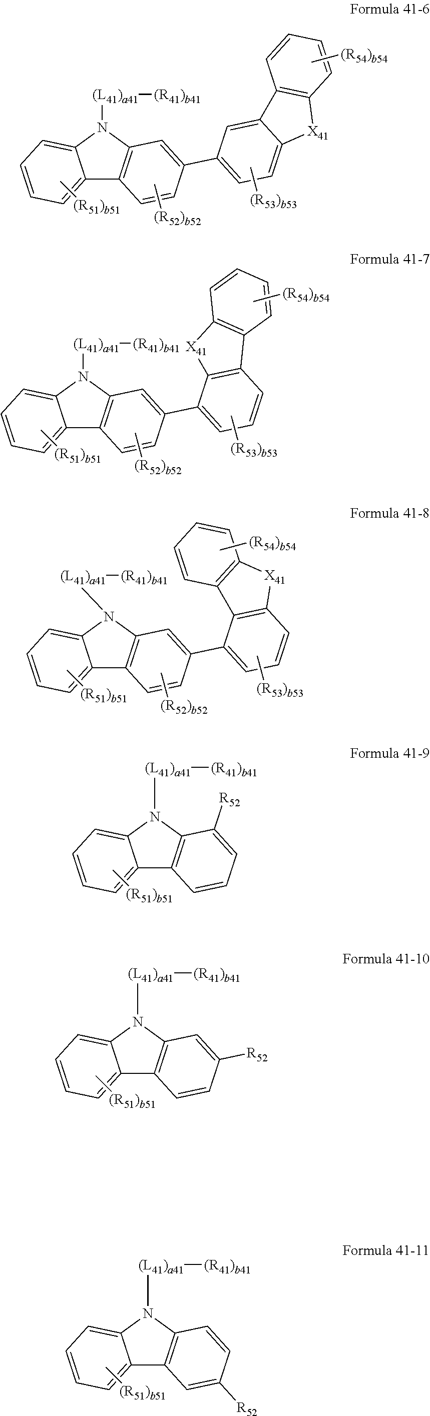

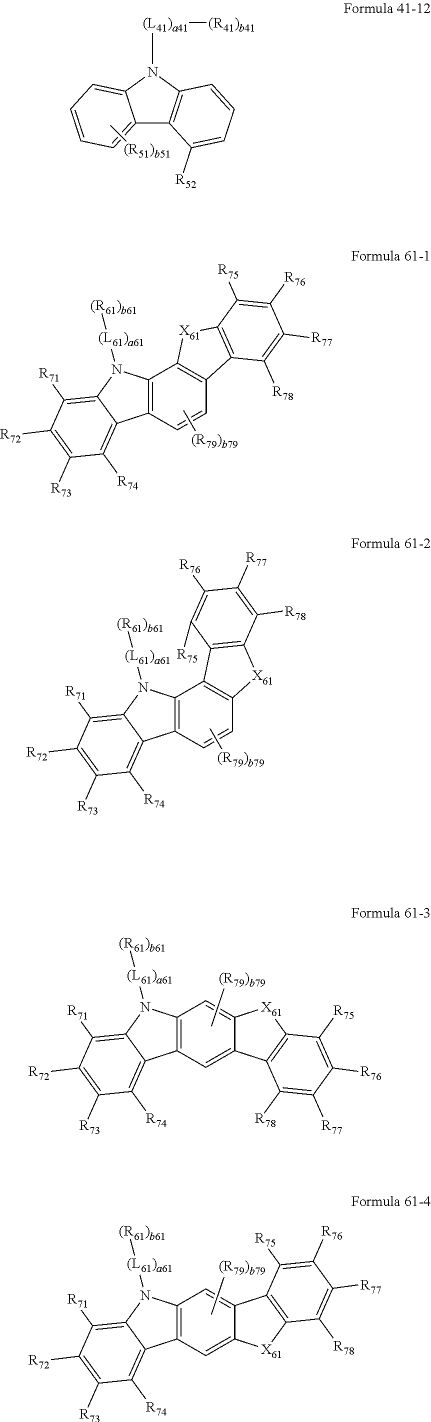

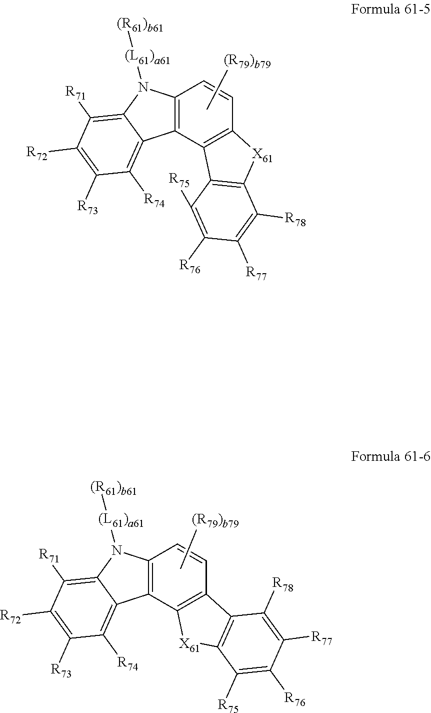

In Formulae 41-1 to 41-12 and 61-1 to 61-6, descriptions for X₄₁, X₆₁, L₄₁, L₄₂, a41, a42, L₆₁, L₆₂, a61, a62, R₄₁ to R₄₄, b41, b42, R₆₁ to R₆₄, b61, b62, R₇₁ to R₇₉ and b79 refer to the above descriptions.

According to one embodiment, the first compound represented by the Formula 41may include one of the following compounds A1 to A16, and the second compound represented by Formula 61 may include one of the following compounds B1 to B4:

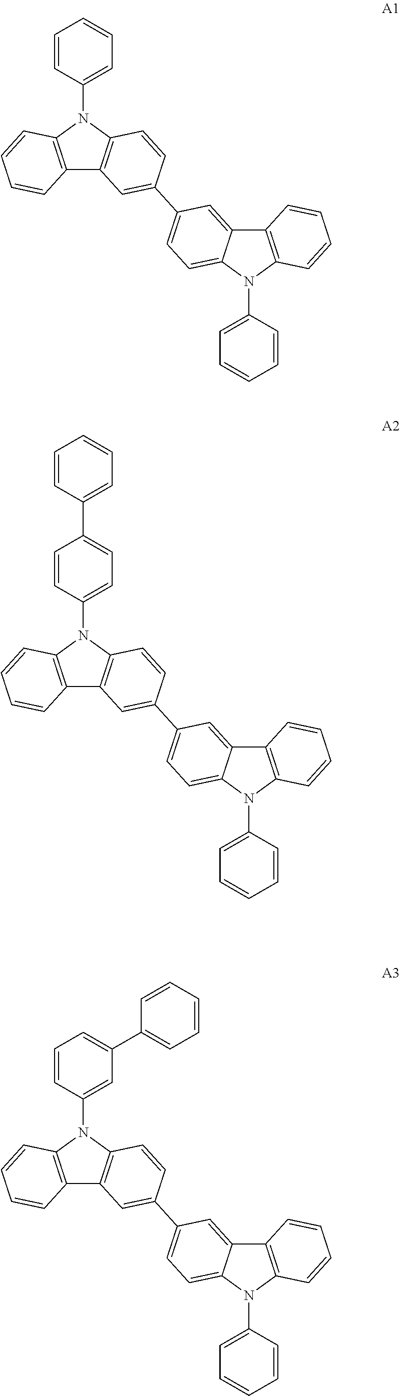 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

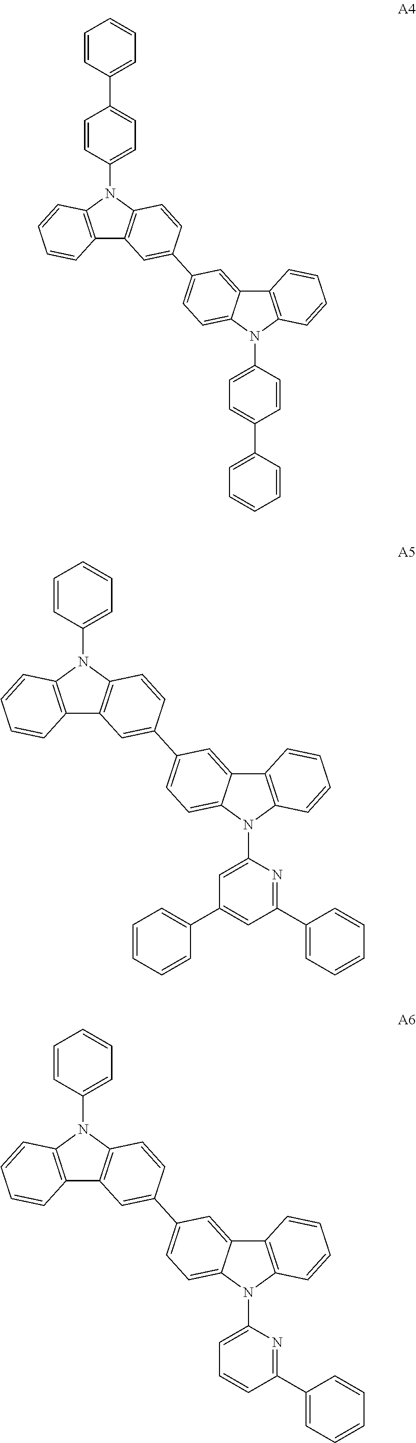 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccccc5)ccc43)n2)cc1]

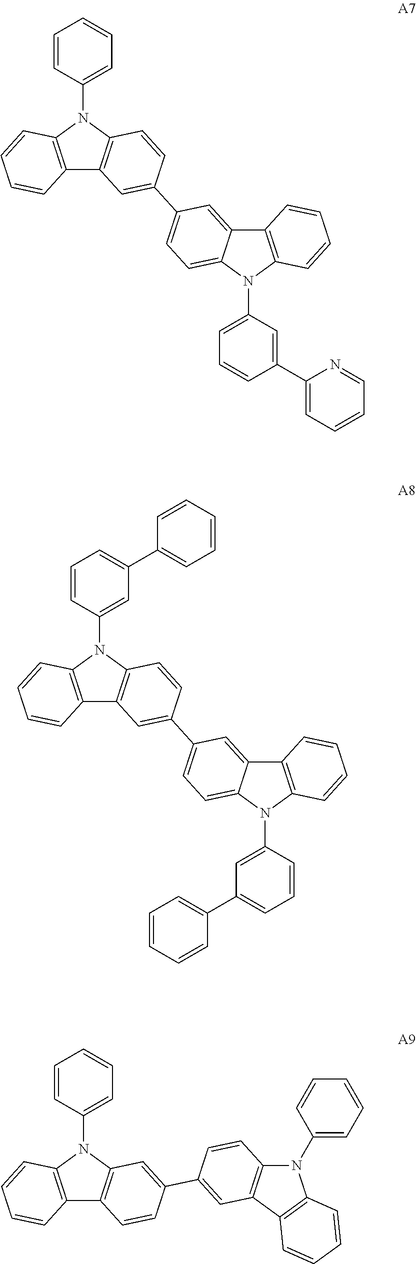 [SMILES: c1ccc(-n2c3ccccc3c3cc(-c4ccc5c(c4)c4ccccc4n5-c4cccc(-c5ccccn5)c4)ccc32)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4ccc5c6ccccc6n(-c6ccccc6)c5c4)ccc32)cc1]

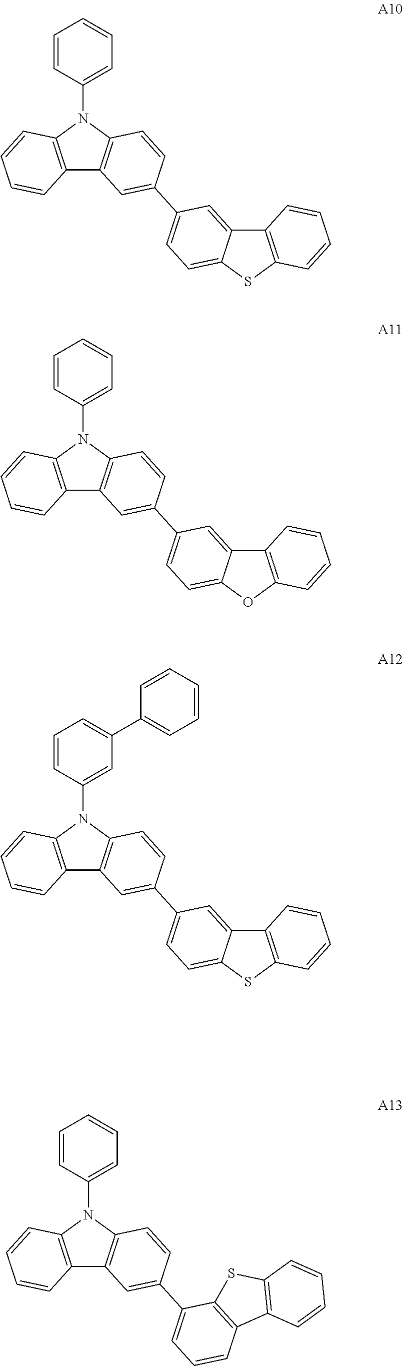 [SMILES: c1ccc(-n2c3ccccc3c3cc(-c4ccc5sc6ccccc6c5c4)ccc32)cc1 ; c1ccc(-n2c3ccccc3c3cc(-c4ccc5oc6ccccc6c5c4)ccc32)cc1 ; c1ccc(-c2cccc(-n3c4ccccc4c4cc(-c5ccc6sc7ccccc7c6c5)ccc43)c2)cc1 ; c1ccc(-n2c3ccccc3c3cc(-c4cccc5c4sc4ccccc45)ccc32)cc1]

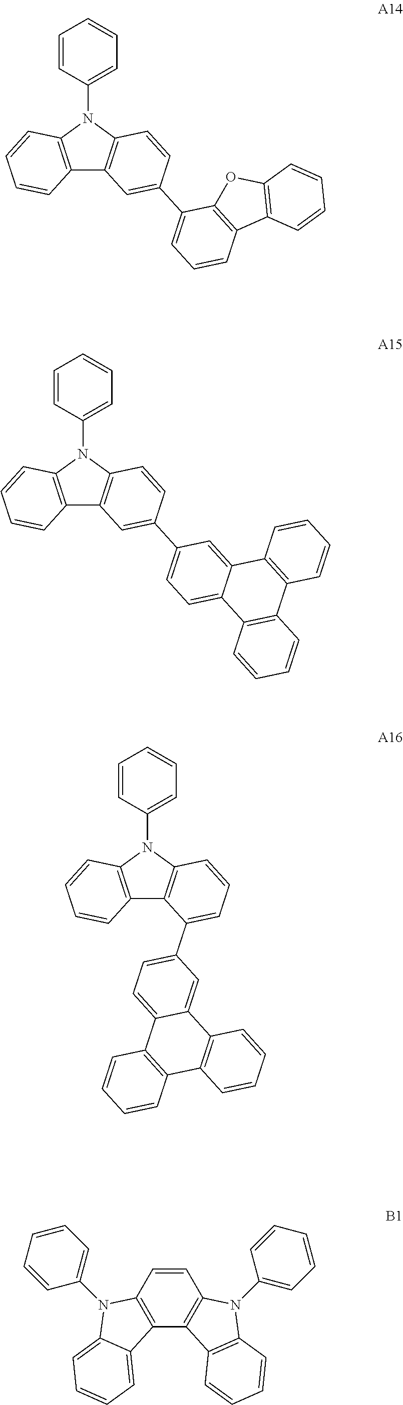 [SMILES: c1ccc(-n2c3ccccc3c3cc(-c4cccc5c4oc4ccccc45)ccc32)cc1 ; c1ccc(-n2c3ccccc3c3cc(-c4ccc5c6ccccc6c6ccccc6c5c4)ccc32)cc1 ; c1ccc(-n2c3ccccc3c3c(-c4ccc5c6ccccc6c6ccccc6c5c4)cccc32)cc1 ; c1ccc(-n2c3ccccc3c3c4c5ccccc5n(-c5ccccc5)c4ccc32)cc1]

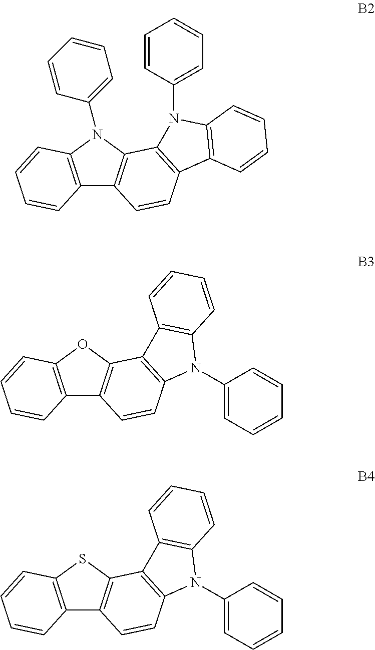 [SMILES: c1ccc(-n2c3ccccc3c3ccc4c5ccccc5n(-c5ccccc5)c4c32)cc1 ; c1ccc(-n2c3ccccc3c3c4oc5ccccc5c4ccc32)cc1 ; c1ccc(-n2c3ccccc3c3c4sc5ccccc5c4ccc32)cc1]

The phosphorescent dopant may include an organometallic compound represented by the following Formula 81:

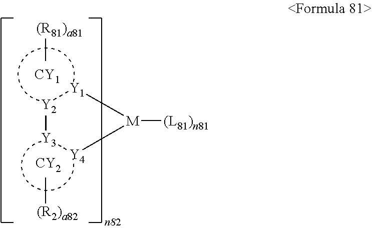

In Formula 81,

M is selected from iridium (Ir), platinum (Pt), osmium (Os), titanium (Ti), zirconium (Zr), hafnium (Hf), europium (Eu), terbium (Tb), and thulium (Tm);

Y₁ to Y₄ are independently carbon (C) or nitrogen (N);

Y₁ and Y₂ are linked to each other through a single bond or a double bond, Y₃ and Y₄ are linked to each other through a single bond or a double bond;

CY₁ and CY₂ are independently selected from benzene, naphthalene, fluorene, spiro-fluorene, indene, pyrrole, thiophene, furan, imidazole, pyrazole, thiazole, isothiazole, oxazole, isoxazole, pyridine, pyrazine, pyrimidine, pyridazine, quinoline, isoquinoline, benzoquinoline, quinoxaline, quinazoline, carbazole, benzoimidazole, benzofuran, benzothiophene, isobenzothiophene, benzooxazole, isobenzooxazole, triazole, tetrazole, oxadiazole, triazine, dibenzofuran or dibenzothiophene, wherein CY₁ and CY₂ are optionally linked to each other through a single bond or an organic linking group;

R₈₁ and R₈₂ are independently selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, —SF₅, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic heterocondensed polycystic group, —N(Q₁)(Q₂), —Si(Q₃)(Q₄)(Q₅), or —B(Q₆)(Q₇);

a81 and a82 are independently selected from integers of 1 to 5;

n81 selected from integers of 0 to 4;

n82 is 1, 2 or 3; and

L₈₁ is selected from a monovalent organic ligand, a divalent organic ligand or a trivalent organic ligand.

The bond between Y₁ and Y₂ and and the bond between Y₃ and Y₄ in Formula 81 may be independently a single bond or a double bond.

The descriptions for the R₈₁ and R₈₂ refer to the description for R₄₁.

The phosphorescent dopant may include at least one of the following compounds PD1 to PD74, but is not limited thereto (the following compound PD1 is Ir(ppy)₃):

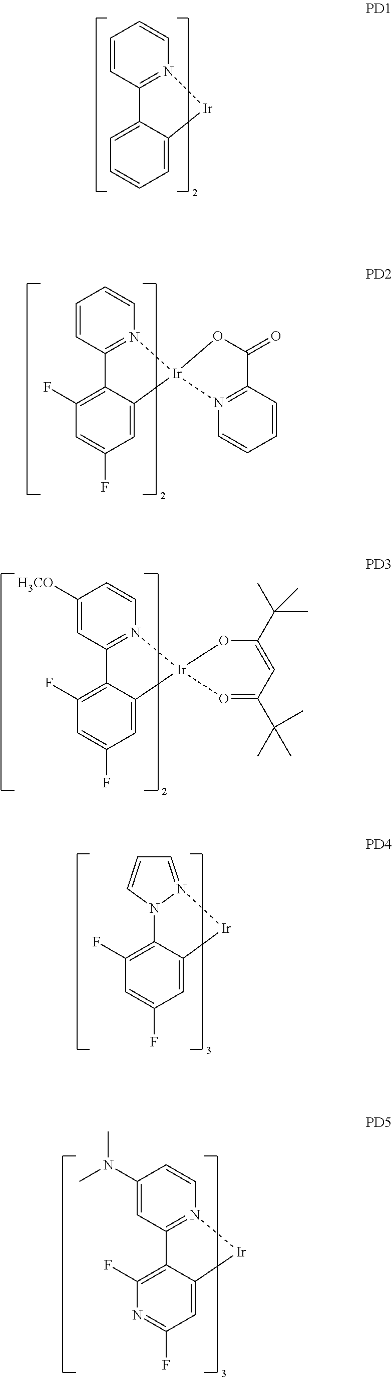

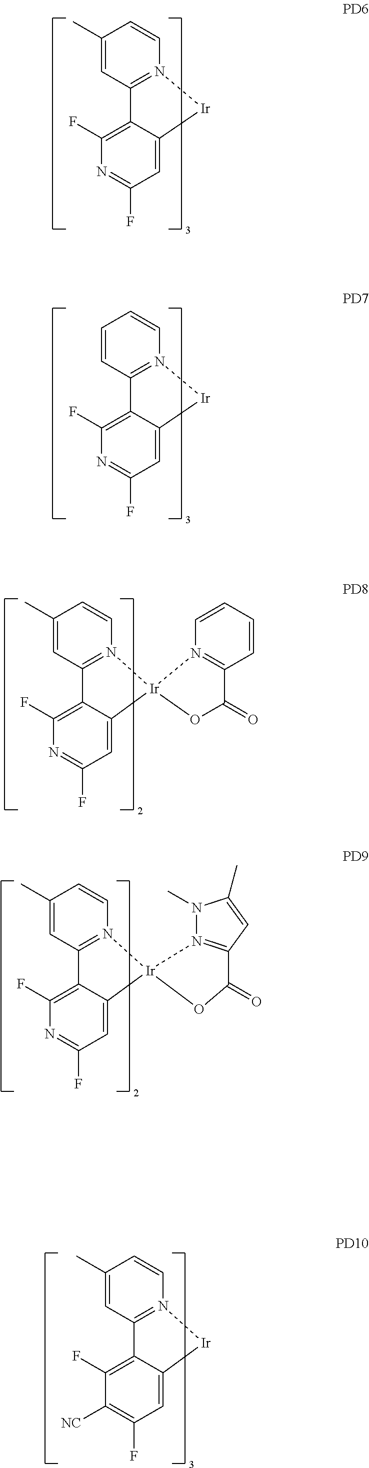

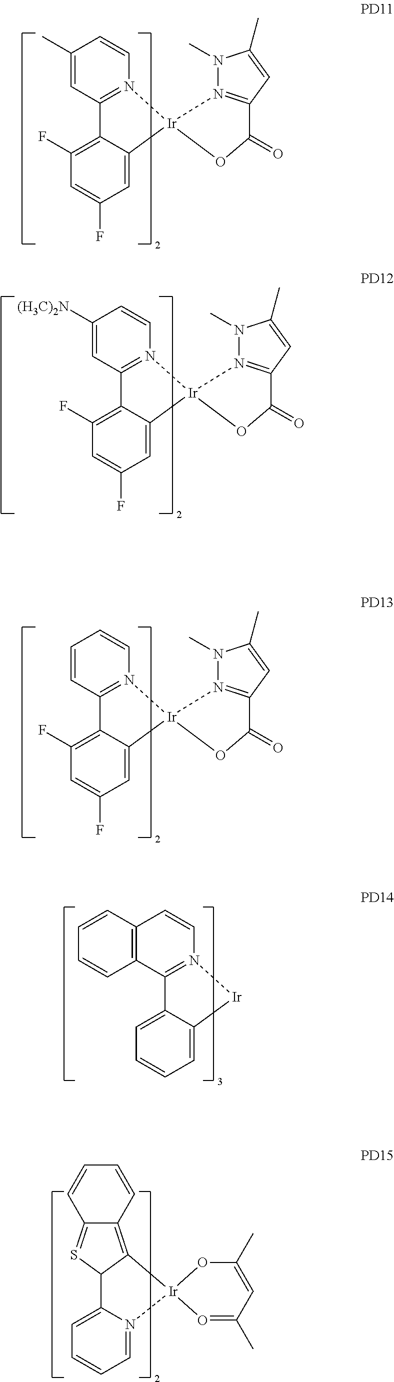

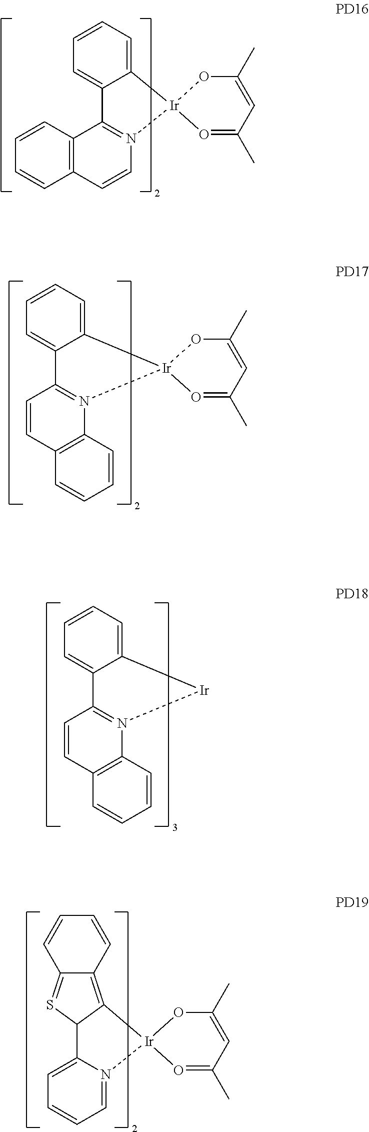

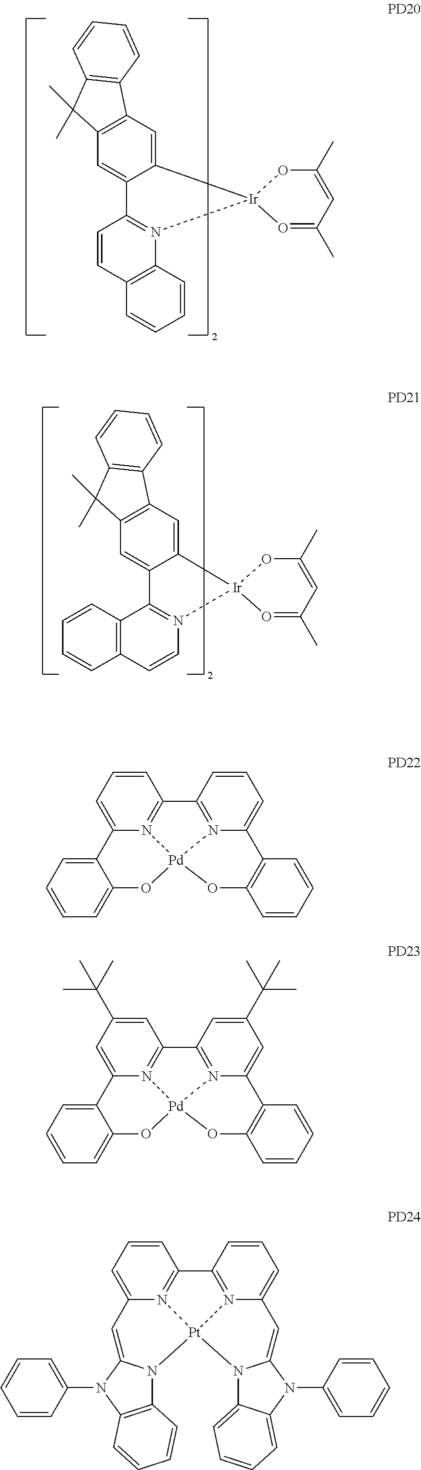

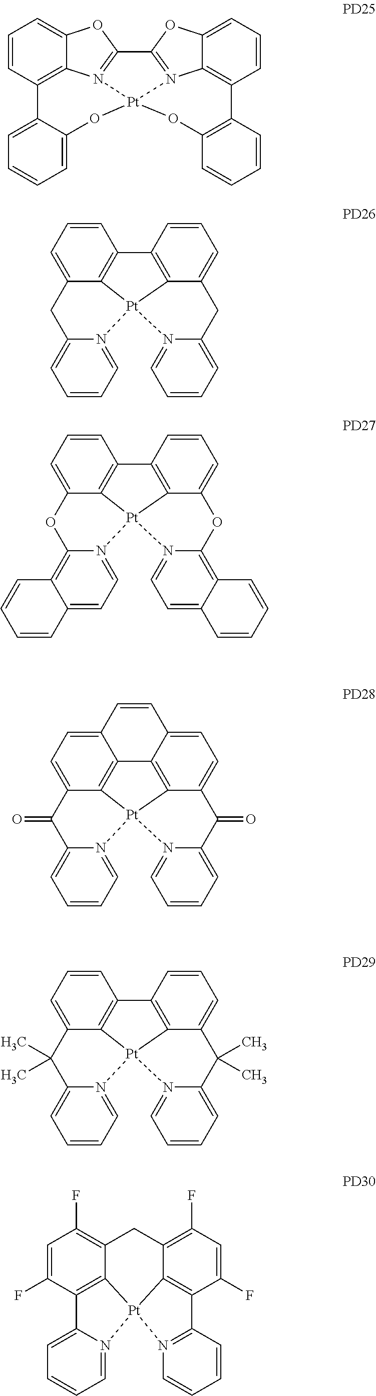

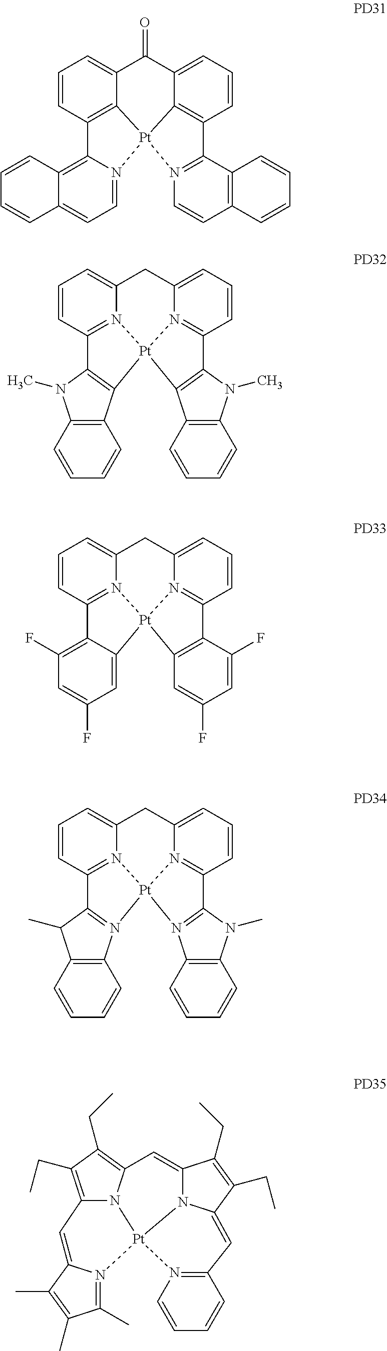

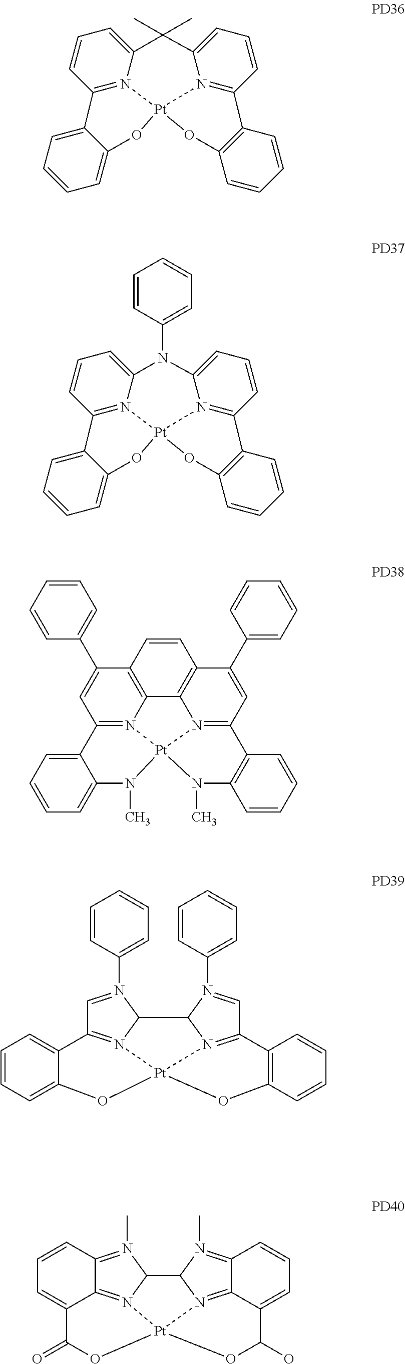

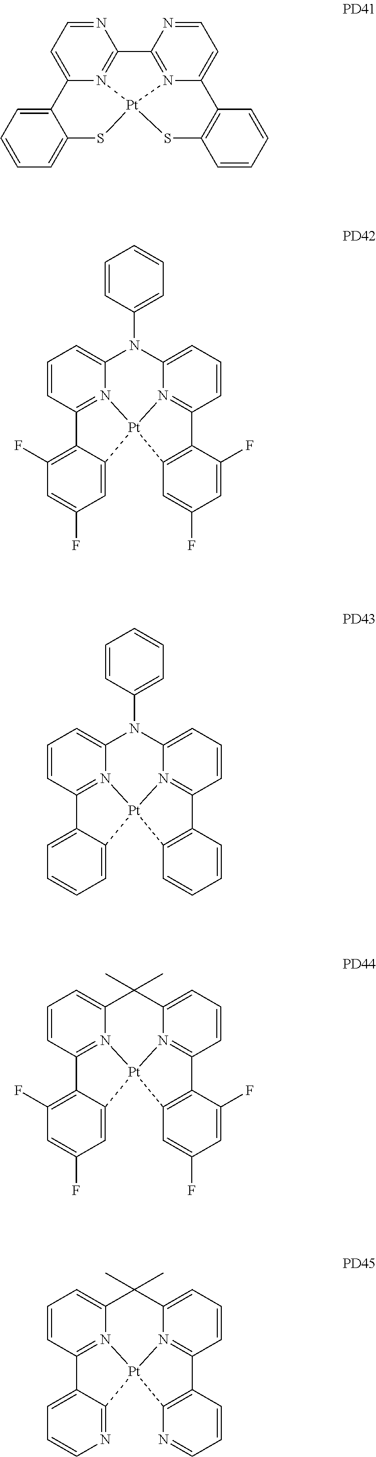

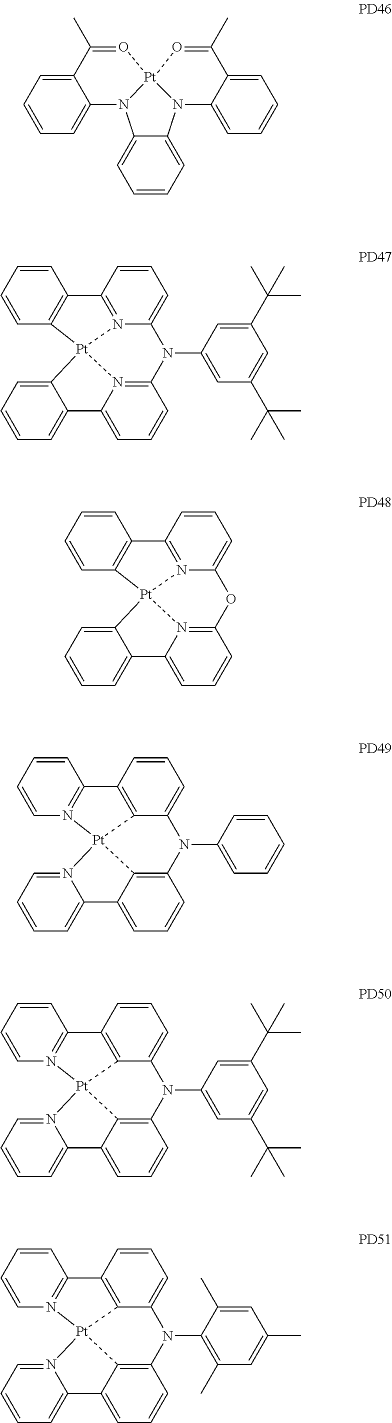

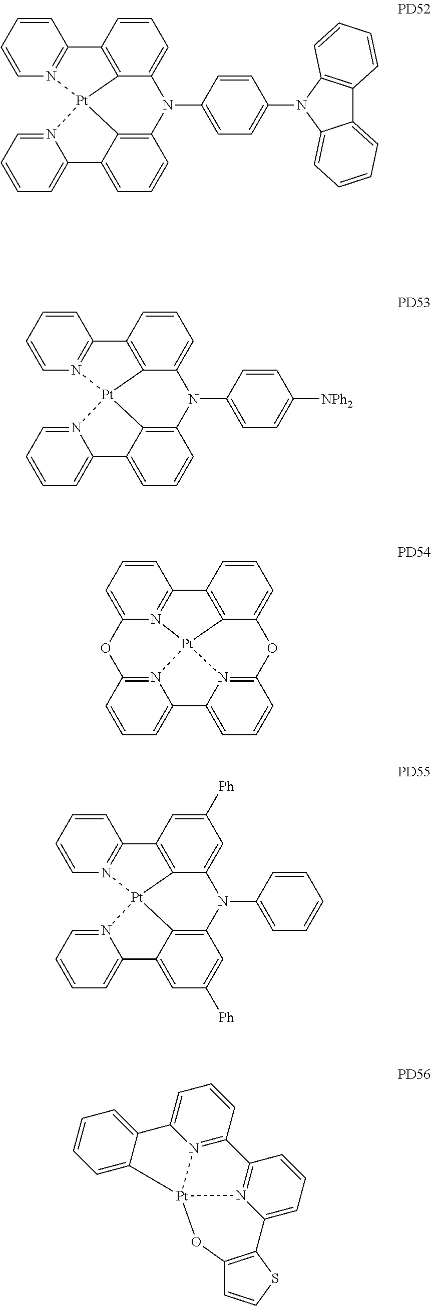

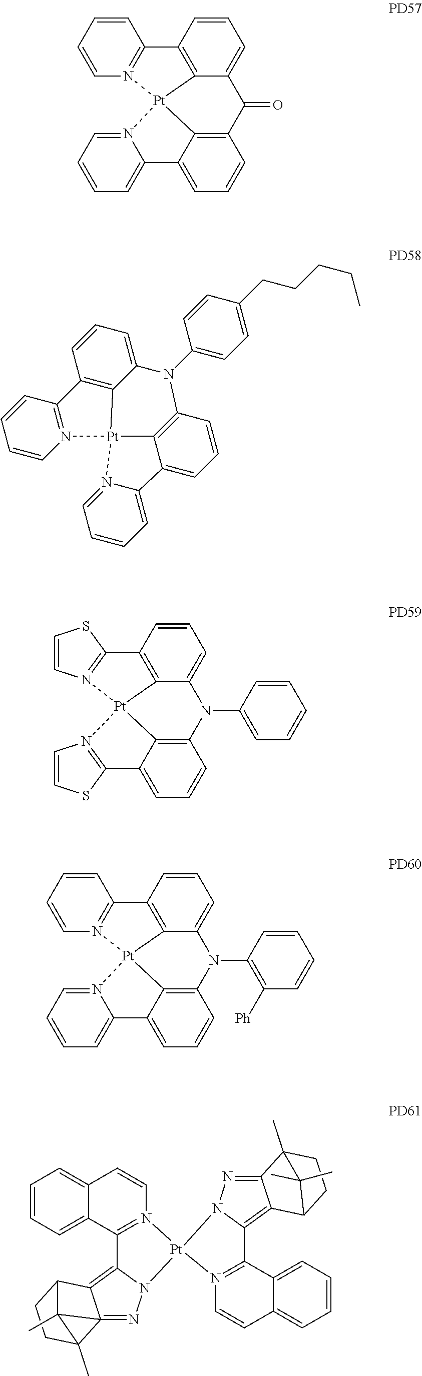

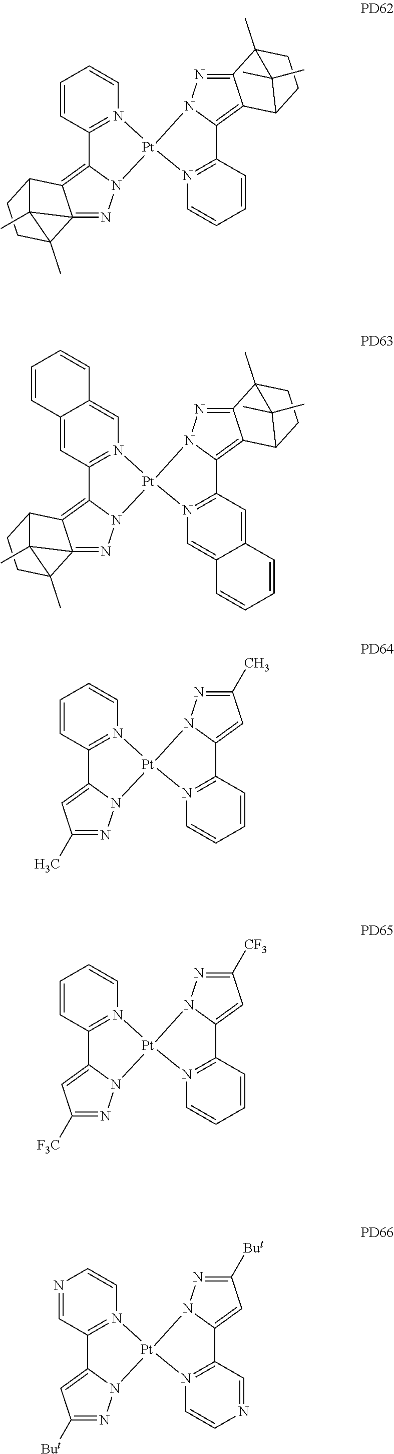

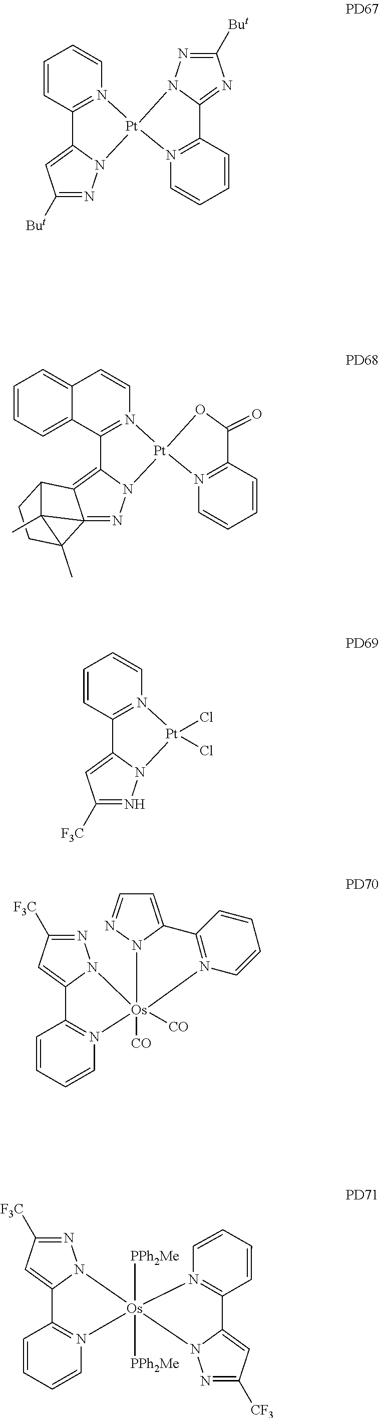

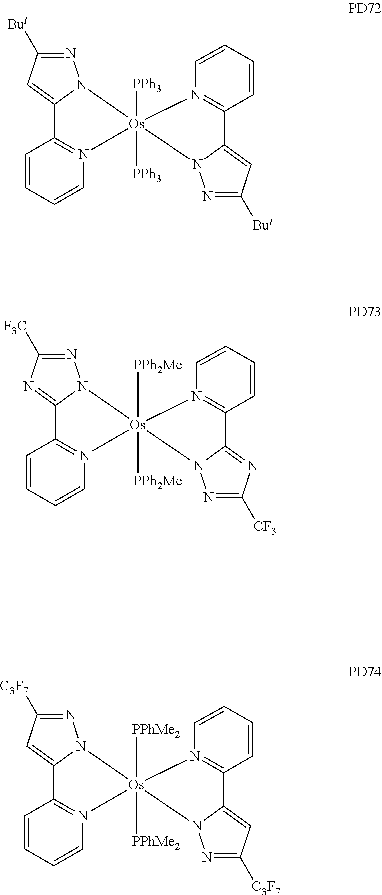

Alternatively, the phosphorescent dopant may include the following compound PtOEP or PhGD:

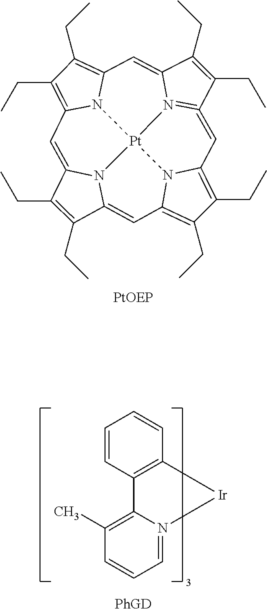

When the emission layer includes a host and a dopant, the content of the dopant may be generally about 0.01 to about 16 parts by weight based on about 100 parts by weight of the host, but is not limited thereto.

The emission layer may be about 100 Å to about 1000 Å thick, for example, about 200 Å to about 600 Å thick. When the emission layer has a thickness within the range, excellent light emitting characteristics may be obtained without substantially increasing a driving voltage.

Next, on the emission layer, an electron transport region is disposed.

The electron transport region may include at least one of a hole blocking layer, an electron transport layer (ETL) and an electron injection layer (EIL).

For example, the electron transport region may have a structure of hole blocking layer/electron transport layer (ETL)/electron injection layer (EIL) or electron transport layer (ETL)/electron injection layer EIL, but is not limited thereto. The electron transport layer (ETL) may have a monolayer structure or a multi-layer structure including more than two materials.

The hole blocking layer, the electron transport layer (ETL) and the electron injection layer (EIL) in the electron transport region are formed referring to the conditions for forming the hole injection layer (HIL).

When the electron transport region includes the hole blocking layer, the hole blocking layer may include, for example, at least one of the following BCP, Bphen and BAlq, but is not limited thereto.

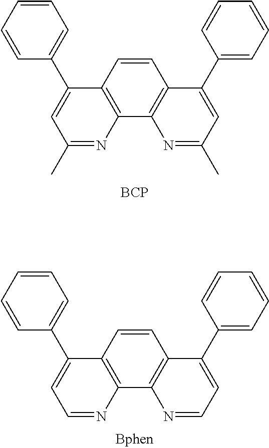

The hole blocking layer may be about 20 Å to about 1000 Å thick, for example about 30 Å to about 300 Å thick. When the hole blocking layer has a thickness within the range, excellent hole blocking characteristics may be obtained without substantially increasing a driving voltage.

The electron transport layer (ETL) may further include at least one of the BCP, the Bphen and the following Alq₃, Balq, TAZ and NTAZ.

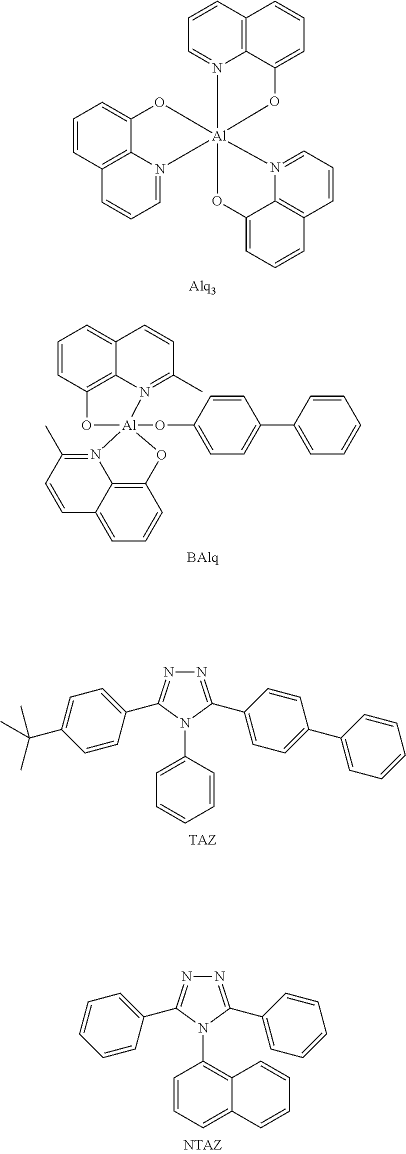

Or, the electron transport layer (ETL) may include at least one of the following compounds ET1 and ET2, but is not limited thereto.

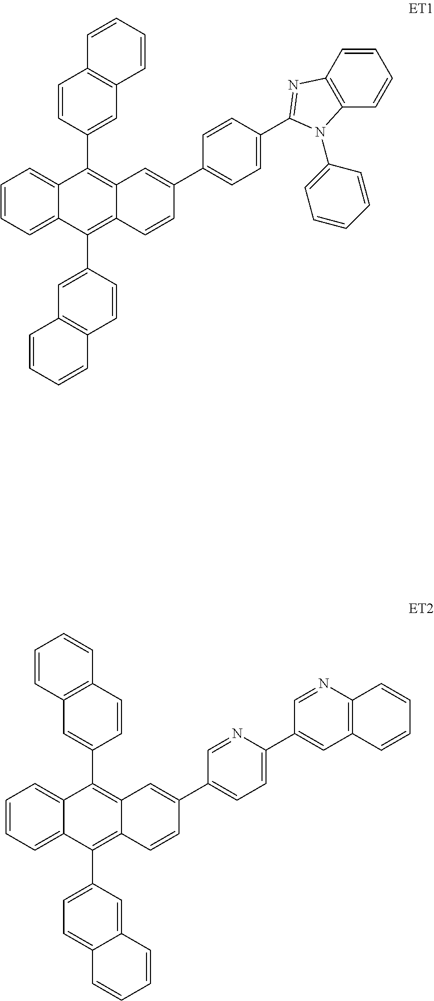 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1]

The electron transport layer (ETL) may be about 100 Å to about 1000 Å thick, for example about 150 Å to about 500 Å thick. When the electron transport layer (ETL) has a thickness within the range, satisfactory electron transport characteristics may be obtained without substantially increasing a driving voltage.

The electron transport layer (ETL) may further include a metal-containing material other than the aforementioned material.

The metal-containing material may include a Li complex. The Li complex may include, for example, the following compound ET-D1 (lithium quinolate, LiQ) or ET-D2.

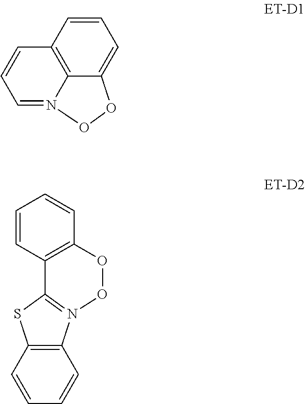

In addition, the electron transport region may include an electron injection layer (EIL) facilitating injection of electrons from the second electrode 19.

The electron injection layer (EIL) may include at least one selected from LiF, NaCl, CsF, Li₂O and BaO.

The electron injection layer (EIL) may be about 1 Å to about 100 Å thick and specifically, about 3 Å to about 90 Å thick. The electron injection layer (EIL) has a thickness within the range, satisfactory electron injection characteristics may be obtained without substantially increasing a driving voltage.

On the organic layer 15, the second electrode 19 is disposed. The second electrode 19 may be a cathode. A material for forming the second electrode 19 may include a metal, an alloy, electrically conductive compound and a combination thereof having a relatively low work function. Specific examples of the material for forming the second electrode 19 may include lithium (Li), magnesium (Mg), aluminum (Al), aluminum-lithium (Al—Li), calcium (Ca), magnesium-indium (Mg—In), magnesium-silver (Mg—Ag), and the like. Or, various modifications of forming the transmissive second electrode 19 by using ITO and IZO and the like may be performed to manufacture a front light emitting element.

Hereinbefore, the organic light emitting device is described referring to FIG. 1, but is not limited thereto.

In the present specification, the C₁-C₆₀ alkyl group refers to a C1 to C60 linear or branched aliphatic hydrocarbon monovalent group, and specific examples may include a methyl group, an ethyl group, a propyl group, an isobutyl group, a sec-butyl group, a tert-butyl group, a pentyl group, an iso-amyl group, a hexyl group, and the like. In the present specification, the C₁-C₆₀ alkylene group refers to a divalent group having the same structure as the C₁-C₆₀ alkyl group.

In the present specification, the C₁-C₆₀ alkoxy group is a monovalent group having Formula, —OA₁₀₁ (wherein, A₁₀₁ is the C₁-C₆₀ alkyl group), and specific examples may be a methoxy group, an ethoxy group, an isopropyloxy group, and the like.

In the present specification, the C₂-C₆₀ alkenyl group has a structure where at least one carbon double bond is included in the middle or terminal end of the C₂-C₆₀ alkyl group, and specific examples may be an ethenyl group, a propenyl group, a butenyl group, and the like. In the present specification, the C₂-C₆₀ alkenylene group refers to a divalent group having the same structure as the C₂-C₆₀ alkenyl group.

In the present specification, the C₂-C₆₀ alkynyl group has a structure where at least one carbon triple bond is included in the middle or terminal end of the C₂-C₆₀ alkyl group, and specific examples may be an ethynyl group, a propynyl group, and the like. In the present specification, the C₂-C₆₀ alkynylene group refers to a divalent group having the same structure as the C₂-C₆₀ alkynyl group.

In the present specification, the C₃-C₁₀ cycloalkyl group refers to a C3 to C₁₀ monovalent saturated hydrocarbon monocyclic group, and examples thereof may be a cyclopropyl group, a cyclobutyl group, a cyclopentyl group, a cyclohexyl group, a cycloheptyl group, and the like. In the present specification, the C₃-C₁₀ cycloalkylene group refers to a divalent group having the same structure as the C₃-C₁₀ cycloalkyl group.

In the present specification, the C₂-C₁₀ heterocycloalkyl group refers to a C2 to C10 monovalent monocyclic group including a ring-forming atom including at least one heteroatom selected from N, O, P and S, and examples thereof are a tetrahydrofuranyl group, a tetrahydrothiophenyl group, and the like. In the present specification, the C₂-C₁₀heterocycloalkylene group refers to a divalent group having the same structure as the C₂-C₁₀ heterocycloalkyl group;

In the present specification, the C₃-C₁₀ cycloalkenyl group refers to a C3 to C10 monovalent monocyclic group having at least one double bond in the ring and not having aromacity, and examples thereof are a cyclopentenyl group, a cyclohexenyl group, a cycloheptenyl group, and the like. In the present specification, the C₃-C₁₀ cycloalkenylene group refers to a divalent group having the same structure as the C₃-C₁₀ cycloalkenyl group.

In the present specification, the C₂-C₁₀ heterocycloalkenyl group refers to a C2 to C10 monovalent monocyclic group including a ring-forming atom including at least one heteroatom selected from N, O, P and S, and includes at least one double bond in the ring. Examples of the C₂-C₁₀ heterocycloalkenyl group are a 2,3-hydrofuranyl group, a 2,3-hydrothiophenyl group, and the like. In the present specification, the C₂-C₁₀ heterocycloalkenylene group refers to a divalent group having the same structure as the C₂-C₁₀ heterocycloalkenyl group.

In the present specification, the C₆-C₆₀ aryl group refers to a monovalent group having a C6 to C60 carbocyclic aromatic system, and C₆-C₆₀ arylene group refers to a divalent group having a C6 to C60 carbocyclic aromatic system. Examples of the C₆-C₆₀ aryl group are a phenyl group, a naphthyl group, an anthracenyl group, a phenanthrenyl group, a pyrenyl group, a chrysenyl group, and the like. When the C₆-C₆₀ aryl group and C₆-C₆₀ arylene group includes 2 or more rings, the 2 or more rings may be fused.

In the present specification, the C₂-C₆₀ heteroaryl group refers to a monovalent group including a ring-forming atom including at least one heteroatom selected from N, O, P and S, and having a C2 to C60 carbocyclic aromatic system, and the C₂-C₆₀ heteroarylene group refers to a divalent group including a ring-forming atom including at least one heteroatom selected from N, O, P and S and having a C2 to C60 carbocyclic aromatic system. Examples of the C₂-C₆₀ heteroaryl group are a pyridinyl group, a pyrimidinyl group, a pyrazinyl group, a pyridazinyl group, a triazinyl group, a quinolinyl group, an isoquinolinyl group, and the like. When the C₂-C₆₀ heteroaryl group and C₂-C₆₀ heteroarylene group include 2 or more rings, the 2 or more rings may be fused.

In the present specification, the C₆-C₆₀ aryloxy group refers to —OA₁₀₂ (wherein, A₁₀₂ is the C₆-C₆₀ aryl group), and the C₆-C₆₀ arylthio group refers to —SA₁₀₃ (wherein, A₁₀₃ is the C₆-C₆₀ aryl group).

In the present specification, the monovalent non-aromatic condensed polycyclic group refers to a monovalent group including condensation of 2 or more rings, and including only carbon as ring-forming atoms (e.g., carbon numbers are 8 to 60), and having non-aromaticity over the entire molecule. Examples of the non-aromatic condensed polycyclic group are a fluorenyl group, and the like. In the present specification, the divalent non-aromatic condensed polycyclic group refers to a divalent group having the same structure as the monovalent non-aromatic condensed polycyclic group.

In the present specification, the monovalent non-aromatic heterocondensed polycyclic group refers to a monovalent group including condensation of 2 or more rings, and including hetero atoms selected from N, O, P and S as well as carbon (e.g., carbon numbers are 2 to 60) as ring-forming atoms, and having non-aromaticity over the entire molecule. The monovalent non-aromatic heterocondensed polycyclic group includes a carbazolyl group, and the like. In the present specification, the divalent non-aromatic heterocondensed polycyclic group refers to a divalent group having the same structure as the monovalent non-aromatic heterocondensed polycyclic group.

In the present specification, at least one substituent of the substituted C₁-C₆₀ alkylene group, the substituted C₂-C₆₀ alkenylene group, the substituted C₂-C₆₀ alkynylene group, the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₂-C₆₀ heteroarylene group, the substituted divalent non-aromatic condensed polycyclic group, the substituted divalent non-aromatic heterocondensed polycyclic group, the substituted C₁-C₆₀ alkyl group, the substituted C₂-C₆₀ alkenyl group, the substituted C₂-C₆₀ alkynyl group, the substituted C₃-C₁₀ cycloalkyl group, the substituted C₂-C₁₀heterocycloalkyl group, the substituted C₃-C₁₀ cycloalkenyl group, the substituted C₂-C₁₀heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic heterocondensed polycystic group may be selected from,

deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀alkoxy group;

a C₁-C₆₀ alkyl group, a C₂-C₆₀ alkenyl group, a C₂-C₆₀ alkynyl group, and a C₁-C₆₀ alkoxy group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C_{10 a} cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, monovalent non-aromatic heterocondensed polycyclic group, —N(Q₁₁)(Q₁₂), —Si(Q₁₃)(Q₁₄)(Q₁₅), and —B(Q₁₆)(Q₁₇);

a C₃-C₅₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, or a monovalent non-aromatic heterocondensed polycyclic group, each of which is substituted with at least one of deuterium, —F, —Cl, —Br, —I, a hydroxyl group, a cyano group; a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic heterocondensed polycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇).

In the present specification, Q₁ to Q₇, Q₁₁ to Q₁₇ and Q₃₁ to Q₃₇ may be independently selected from hydrogen, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₂-C₆₀ heteroaryl group, a monovalent non-aromatic condensed polycyclic group, and a monovalent non-aromatic heterocondensed polycyclic group.

For example, at least one substituent of the substituted C₁-C₆₀ alkylene group, the substituted C₂-C₆₀ alkenylene group, the substituted C₂-C₆₀ alkynylene group, the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₂-C₆₀ heteroarylene group, the substituted divalent non-aromatic condensed polycyclic group, the substituted divalent non-aromatic heterocondensed polycyclic group, the substituted C₁-C₆₀ alkyl group, the substituted C₂-C₆₀ alkenyl group, the substituted C₂-C₆₀ alkynyl group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic heterocondensed polycyclic group may be selected from

deuterium, —F, —Cl, —Br, —I, a hydroxyl group, a cyano group, a nitro group, an amino group, ant amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group and C₁-C₆₀ alkoxy group;.

a C₁-C₆₀ alkyl group, a C₂-C₆₀ alkenyl group, a C₂-C₆₀ alkynyl group, and a C₁-C₆₀ alkoxy group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C_{10 a} cycloalkenyl group, a C₂-C₁₀ heterocycloalkenyl group, a C₆-C₆₀ aryl group, a C₆-C₆₀ aryloxy group, a C₆-C₆₀arylthio group, a C₂-C₆₀ heteroaryl group, a monovalent non-aromatic condensed polycyclic group, a monovalent non-aromatic heterocondensed polycyclic group, —N(Q₁₁)(Q₁₂), —Si(Q₁₃)(Q₁₄)(Q₁₅), and —B(Q₁₆)(Q₁₇);

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, pyrrolyl group, an imidazolyl group, a pyrazolyl group, a yridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, phenanthrolinyl group, a phenazinyl group, a benzooxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyrimidinyl group and an imidazopyridinyl group, each of which is substituted with at least one of a cyclopentyl group, a cyclohexyl group, a cycloheptyl group, a cyclooctyl group, a cyclopentenyl group, a cyclohexenyl group, a cycloheptenyl group, a phenyl group, a naphthyl group, an anthracenyl group, a pyrenyl group, a phenanthrenyl group, a fluorenyl group, a carbazolyl group, a benzocarbazolyl group, dibenzocarbazolyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group, and a quinazolinyl group;

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzooxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyrimidinyl group and an imidazopyridinyl group, each of which is substituted with at least one of a cyclopentyl group, a cyclohexyl group, a cycloheptyl group, a cyclooctyl group, a cyclopentenyl group, a cyclohexenyl group, a cycloheptenyl group, a phenyl group, a naphthyl group, an anthracenyl group, a pyrenyl group, a phenanthrenyl group, a fluorenyl group, a carbazolyl group, a benzocarbazolyl group, a dibenzocarbazolyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a Cinnolinyl group, and a quinazolinyl group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group and C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic heterocondensed polycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇);

wherein the Q₁ to Q₇, Q₁₁ to Q₁₇ and Q₃₁ to Q₃₇ are independently a phenyl group, a pentalenyl group, an indenyl group, a naphthyl group, an azulenyl group, a heptalenyl group, an indacenyl group, an acenaphthyl group, a fluorenyl group, a spiro-fluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzooxazolyl group, a benzoimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyrimidinyl group and an imidazopyridinyl group, each of which is substituted with at least one of hydrogen, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclooctyl group, a cyclopentenyl group, a cyclohexenyl group, a cycloheptenyl group, a phenyl group, a naphthyl group, an anthracenyl group, a pyrenyl group, a phenanthrenyl group, a fluorenyl group, a carbazolyl group, a benzocarbazolyl group, a dibenzocarbazolyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a Cinnolinyl group, and a quinazolinyl group, but are not limited thereto.

Hereinafter, a compound and an organic light emitting device according to one embodiment of the present invention are specifically illustrated referring to Synthesis Examples and Examples, but the present invention is not limited to the following Synthesis Examples and Examples. In the following Synthesis Examples, when “‘B’ is used instead of ‘A’”, the amounts of ‘A’ and ‘B’ are the same as based on a mole equivalent.

Hereinafter, a starting material and a reaction material used in Examples and Synthesis Examples were purchased from Sigma-Aldrich Co. Ltd. or TCI Inc. unless there was particularly mentioned.

### EXAMPLE

### Synthesis Example 1

Synthesis of Compound 8

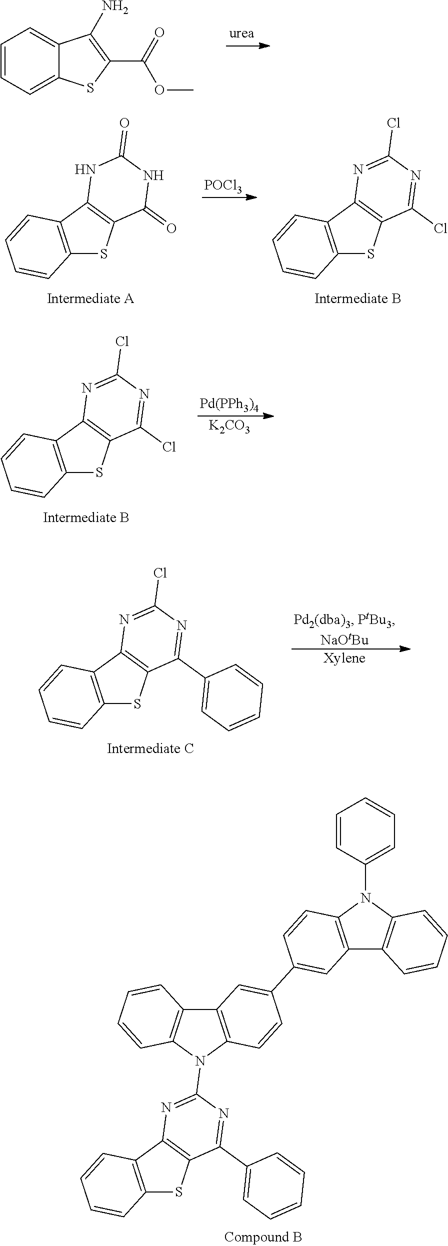

Synthesis of Intermediate A (benzo-1H-thieno [3,2-d]pyrimidine-2,4-dione)

A mixture of 3-aminobenzo-thiophene-2-carboxylate (47.5 g, 0.23 mol) and urea (79.4 g, 1.15 mol) was agitated at 200° C. for 2 hours in a 2000 mL round flask. The reaction mixture at the high temperature was cooled down to room temperature and poured into a sodium hydroxide solution, the reactant obtained by filtering and removing an impurity therein was acidized (HCl, 2N) to obtain a precipitate, and the precipitate was dried, obtaining an intermediate A. (35 g, 75%)

calcd. C₁₀H₆N₂O₂S: C, 55.04; H, 2.77; N, 12.84; O, 14.66; S, 14.69; found: C. 55.01; H, 2.79; N, 12.81; O, 14.69; S, 14.70

Synthesis of Intermediate B (benzo-2,4-dichloro-thieno[3,2-d]pyrimidine)

A mixture of the intermediate A (benzo-1H-thieno[3,2-d]pyrimidine-2,4-dione) (35 g, 0.16 mol) and 600 mL of phosphorus oxychloride was agitated under a reflux for 6 hours in a 1000 mL round flask. The reaction mixture was cooled down to room temperature, and ice/water was poured thereinto to produce a precipitate while the reaction mixture was agitated. The obtained reactant was filtered, obtaining an intermediate B (benzo-2,4-dichloro-thieno [3,2-d]pyrimidine) (35 g, 85%, a white solid). Elemental analysis and NMR analysis results of the intermediate B are as follows.

calcd. C₁₀H₄Cl₂N₂S: C, 47.08; H, 1.58; Cl, 27.79; N, 10.98; S, 12.57; found: C, 47.03; H, 1.61; Cl, 27.81; N, 10.98; S, 12.60

300 MHz (CDCl₃, ppm): 7.63 (t, 1H), 7.76 (t, 4H), 7.95 (d, 1H), 8.53 (d, 1H)

Synthesis of Intermediate C

30.0 g (117.6 mmol) of the intermediate B, 15.77 g (129.4 mmol) of phenylboronic acid; 40.63 g (294.0 mmol) of potassium carbonate, and 6.79 g (5.88 mmol) of Pd(PPh₃)₄(tetrakis(triphenylphosphine)palladium (0)) were added to 600 mL of 1,4-dioxane and 200 mL of water in a 2000 mL round flask, and the mixture was heated under reflux for 12 hours under a nitrogen atmosphere. The obtained mixture was added to 1500 mL of methanol, and a solid crystallized therein, dissolved in monochlorobenzene, filtered with silica gel/Celite, and then, recrystallized with methanol after removing an organic solvent in an appropriate amount, obtaining an intermediate C (20.94 g, 60%). Elemental analysis results of the intermediate C are as follows.

calcd. C₁₆H₉ClN₂S: C, 64.75, H, 3.06; Cl, 11.95; N, 9.44; S, 10.80; found: C, 64.77; H, 3.05; Cl, 11.97; N, 9.47; S, 10.78

Synthesis of Compound 8

20.0 g (67.39 mmol) of the intermediate C, 30.28 g (74.13 mmol) of 9-phenyl-9H,9′H-3,3′-bicarbazole, 19.43 g (202.18 mmol) of sodium t-butoxide, 6.2 g (6.7 mmol) of Pd(dba)₂, and 6.54 mL (50% in toluene) of tri t-butylphosphine were added to 400 mL of xylene in a 1000 mL round flask, and the mixture was heated under reflux for 15 hours under a nitrogen atmosphere

The obtained mixture was added 1000 mL of methanol, and a solid crystallized therein was filtered, dissolved in dichlorobenzene, and filtered using silica gel/Celite, followed by removing an appropriate amount of the organic solvent and recrystallization with methanol to obtain the compound 8 (29.3 g, yield: 65%). Elemental analysis results of the compound 8 are as follows.

calcd. C₄₆H₂₈N₄S: C, 82.61; H, 4.22; N, 8.38; S, 4.79; found: C, 82.63; H, 4.21; N, 8.36; S, 4.80

### Synthesis Example 2

Synthesis of Compound 21

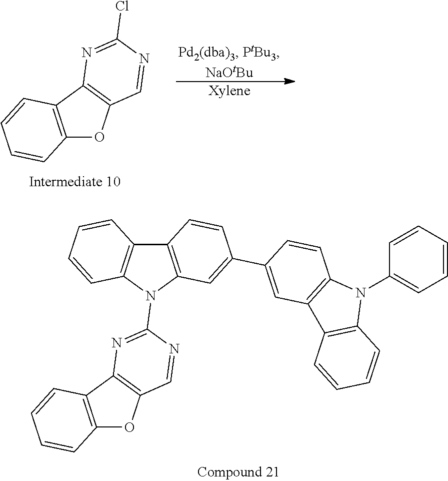 [SMILES: Clc1ncc2oc3ccccc3c2n1 ; "Pd2(dba)3, PtBu3,NaOtBu" ; "Xylene" ; c1ccc(-n2c3ccccc3c3cc(-c4ccc5c6ccccc6n(-c6ncc7oc8ccccc8c7n6)c5c4)ccc32)cc1]

14.5 g (70.87 mmol, UMT Co., Ltd., Duksan Techopia Co., Ltd.) of the intermediate 10, 31.84 g (77.95 mmol) of 9′-phenyl-9H,9′H-2,3′-bicarbazole, 20.43 g (212.6 mmol) of sodium t-butoxide, 6.49 g (7.09 mmol) of Pd(dba)₂, and 6.88 mL (50% in toluene) of tri t-butylphosphine were added to 400 mL of xylene in a 1000 mL round flask, and the mixture was heated under reflux for 15 hours under a nitrogen atmosphere. The obtained mixture was added to 1000 mL of methanol, and a solid crystallized therein was filtered, dissolved in dichlorobenzene, and filtered using silica gel/Celite, followed by removing an appropriate amount of the organic solvent and recrystallization with methanol to obtain the compound 21 (27.8 g, yield: 68%). Elemental analysis results of the compound 21 are as follows.

calcd. C₄₀H₂₄N₄O: C, 83.31; H, 4.20; N, 9.72; O, 2.77; found: C, 83.30; H, 4.21; N, 9.76; O, 2.78

300 MHz (CDCl₃, ppm): 7.28-7.59 (m, 8H), 7.59-7.80 (m, 7H), 7.87 (dd, 1H), 8.13 (d, 1H), 8.19 (d, 1H), 8.26 (d, 1H), 8.39 (d, 1H), 8.57 (d, 1H), 8.93 (d, 1H), 9.12 (s, 1H), 9.34 (s, 1H)

### Synthesis Example 3

Synthesis of Compound 26

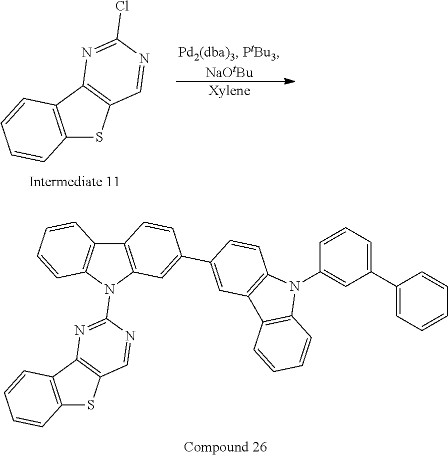 [SMILES: Clc1ncc2sc3ccccc3c2n1 ; "Pd2(dba)3, PtBu3,NaOtBu" ; "Xylene" ; c1ccc(-c2cccc(-n3c4ccccc4c4cc(-c5ccc6c7ccccc7n(-c7ncc8sc9ccccc9c8n7)c6c5)ccc43)c2)cc1]

16.0 g (72.60 mmol, UMT Co., Ltd., Duksan Techopia Co., Ltd.) of the intermediate 11, 38.70 g (79.86 mmol) of 9′-([1,1′-biphenyl]-3-yl)-9H,9′H-2,3′-bicarbazole; 20.93 g (217.8 mmol) of sodium t-butoxide, 6.65 g (7.26 mmol) of Pd(dba)₂, and 7.04 mL (50% in toluene) of tri-t-butylphosphine were added to 400 mL of xylene in a 1000 mL round flask, and the mixture was heated under reflux for 15 hours under a nitrogen atmosphere. The obtained mixture was added to 1000 mL of methanol, and a solid crystallized therein was filtered, dissolved in dichlorobenzene, and filtered using silica gel/Celite, followed by removing an appropriate amount of the organic solvent and recrystallization with methanol to obtain the compound 26 (27.8 g, yield: 68%). Elemental analysts results of the compound 26 are as follows.

calcd. C₄₆H₂₈N₄S: C, 82.61; H: 4.22; N, 8.38; S, 4.79; found: C, 82.64; H, 4.24; N, 8.36; S, 4.78

### Synthesis Example 4

Synthesis of Compound29

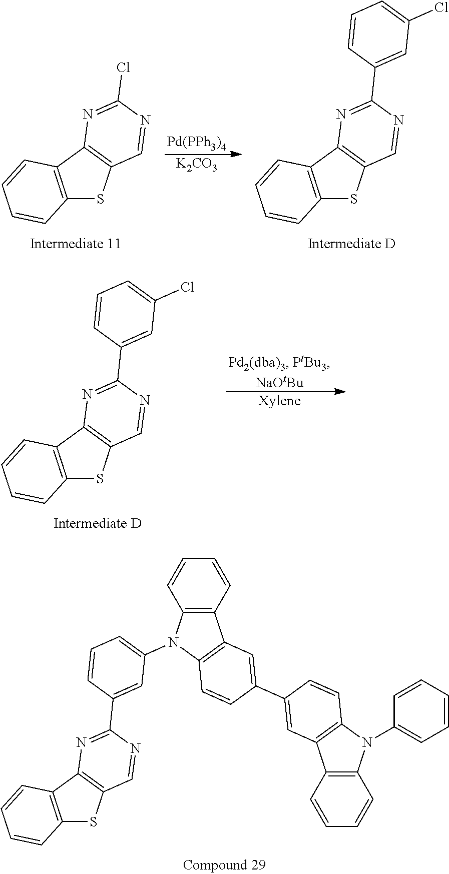

Synthesis of Intermediate D

30.0 g (135.94 mmol) of the intermediate 11, 19.07 g (156.37 mmol) of (3-chlorophenyl)boronic acid, 37.58 g (271.83 mmol) of potassium carbonate, and 7.85 g (6.80 mmol) of Pd(PPh₃)₄ (tetrakis(triphenylphosphine)palladium (0)) were added to 800 mL of 1,4-dioxane in a 2000 mL round flask, and the mixture was heated under reflux for 12 hours under a nitrogen atmosphere. The obtained mixture was added lo 2000 mL of methanol, and a solid crystallized therein was filtered, dissolved in monochlorobenzene, and filtered using silica gel/Celite, followed by removing an appropriate amount of the organic solvent and recrystallization with methanol to obtain the intermediate D (22.19 g, 55%).

Synthesis of Compound 29

18.0 g (60.65 mmol) of the intermediate D, 27.25 g (66.72 mmol) of 9-phenyl-9H,9′H-3,3′-bicarbazole, 17.49 g (181.96 mmol) of sodium t-butoxide, 5.55 g (6.07 mmol) of Pd(dba)₂, and 5.88 mL (50% in toluene) of tri t-butylphosphine were added to 350 mL of xylene in a 1000 mL found flask, and the mixture was heated under reflux for 15 hours under a nitrogen atmosphere. The obtained mixture was added to 1000 mL of methanol, and a solid crystallized therein was filtered, dissolved in dichlorobenzene, and filtered using silica gel/Celite, followed by removing an appropriate amount of the organic solvent and recrystallization with methanol to obtain the compound compound 29 (23.1 g, yield: 57%). Elemental analysis results of the compound 29 are as follows.

calcd. C₄₆H₂₈N₄S: C, 82.61; H, 4.22; N, 8.38; S, 4.79; found: C, 82.62; Hs 4.21; N, 8.39; S, 4.77

### Evaluate on Example 1

Evaluation of HOMO, LUMO, and triplet (T1) Energy Levels of Synthesized Compounds

Table

The HOMO, LUMO and T1 energy levels of the synthesized compounds were evaluated according to Table 1 and the results are shown in Table 2.

TABLE 1

| | |
|---|---|
| HOMO | Each of the compounds was diluted in CHCl₃ to a concentra- |
| energy | tion of 1 × 10⁻⁵ M, and then UV absorption spectra |
| level | thereof were measured at room temperature using a spectrom- |
| evaluation | eter (Shimadzu UV-350 Spectrometer). A HOMO energy |
| method | level of the compound was calculated based on the |
| | optical band gap (Eg) of the absorption spectrum edge. |
| LUMO | A potential (V)-current (A) plot of each of the compounds was |
| energy | obtained using cyclic voltammetry (CV) (Electrolyte: 0.1M |
| level | Bu₄NClO₄/Solvent: CH₂Cl₂/Electrode: 3-electrode system |
| evaluation | (working electrode: GC, reference electrode: Ag/AgCl, |
| method | auxiliary electrode: PT)), and a LUMO energy of the |
| | compound was calculated based on the reduction onset |
| | potential in the potential-current plot. |
| T1 energy | A mixture of each of the compounds and toluene (prepared by |
| level | dissolving 1 mg of the compound in 3 cc of toluene) was put |
| evaluation | in a quartz cell, which was then placed in liquid nitrogen |
| method | (77K) for photoluminescence spectroscopy. Photo- |
| | luminescence spectra of the compounds were measured |
| | using a photoluminescence spectrometer, and then |
| | compared with those at room temperature to analyze |
| | only peaks appearing at low temperature. A T1 energy |
| | level of each of the compounds was calculated based |
| | on the low-tempeature peaks. |

TABLE 2

| Compound No. | HOMO(eV) | LUMO(eV) | T1 energy level (eV) |
|---|---|---|---|
| compound 6 | −5.006 | −2.016 | 2.409 |
| compound 8 | −5.022 | −1.927 | 2.489 |
| compound 17 | −5.039 | −1.859 | 2.568 |
| compound 18 | −5.096 | −1.864 | 2.687 |
| compound 19 | −5.101 | −1.898 | 2.671 |
| compound 20 | −5.045 | −1.857 | 2.569 |
| compound 21 | −5.103 | −1.860 | 2.688 |
| compound 22 | −4.924 | −1.845 | 2.764 |
| compound 23 | −5.058 | −1.832 | 2.885 |
| compound 24 | −5.057 | −1.828 | 2.604 |
| compound 25 | −5.117 | −1.829 | 2.708 |
| compound 26 | −5.131 | −1.871 | 2.690 |
| compound 27 | −5.061 | −1.825 | 2.605 |
| compound 28 | −5.125 | −1.826 | 2.708 |
| compound 29 | −4.938 | −1.808 | 2.814 |
| compound 30 | −5.067 | −1.800 | 2.882 |

From the Table 2, it was confirmed that the synthesized compounds had high triplet energy and wide bandgap and thus had electrical characteristics appropriate for a material for an organic light emitting device.

### Example 1

A glass substrate with a 1500 Å-thick ITO (Indium tin oxide) electrode (first electrode, anode) was cut to a size of 50 mm×50 mm×0.5 mm, washed by sonication in distilled water. Then, the substrate was ultrasonic wave-cleaned with a solvent such as isopropyl alcohol, acetone, methanol and the like and dried, then, moved to a plasma cleaner and cleaned by using an oxygen plasma for 5 minutes, and then, moved to a vacuum depositor. Then, a 600 Å-thick hole injection layer was formed on the ITO electrode of the glass substrate by vacuum-depositing m-MTDATA at a deposition rate of 1 Å/sec, and a 300 Å-thick hole transport layer was formed on the hole injection layer by vacuum-depositing α-NPB at a deposition rate of 1 Å/sec. Subsequently, a 400 Å-thick emission layer was formed on the hole transport layer by codepositing Ir(ppy)₃ (dopant) and the compound 8 (host) at each deposition rate of 0.1 Å/sec and 1 Å/sec. On the emission layer, a 50 Å-thick hole blocking layer was formed by vacuum-depositing BAlq at a deposition rate of 1 Å/sec, and on the hole blocking layer, 300 Å-thick electron transport layer was formed by vacuum-depositing Alq₃. LiF 10 Å (electron injection layer EIL) and Al 2000 Å (cathode) were sequentially vacuum-deposited on the electron transport layer to manufacture an organic light emitting device.

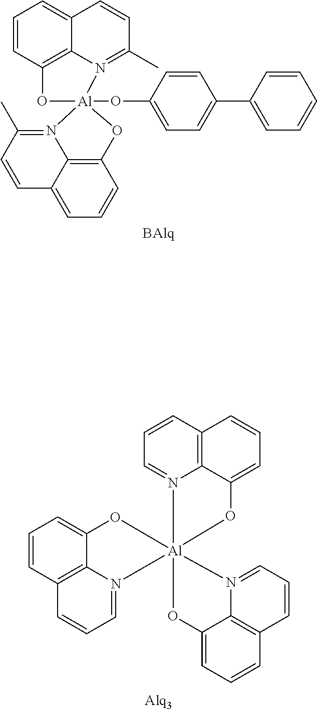

### Example 2

An organic light emitting device was manufactured according to the same method as Example 1 except for using the compound 21 instead of the compound 8 as a host to form an emission layer.

### Example 3

An organic light emitting device was manufactured according to the same method as Example 1 except for using the compound 26 instead of the compound 8 as a host to form an emission layer.

### Example 4

An organic light emitting device was manufactured according to the same method as Example 1 except for using the compound 29 instead of the compound 8 as a host to form an emission layer.

Comparative Example 1

An organic light emitting device was manufactured according to the same method as Example 1 except for using the compound A instead of the compound 9 as a host to form an emission layer.

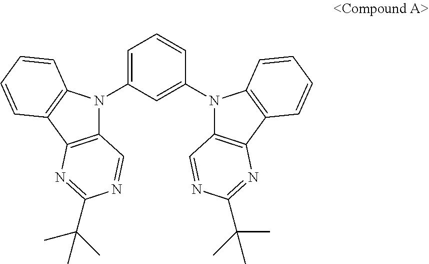 [SMILES: CC(C)(C)c1ncc2c(n1)c1ccccc1n2-c1cccc(-n2c3ccccc3c3nc(C(C)(C)C)ncc32)c1]

Comparative Example 2

An organic light emitting device was manufactured according to the same method as Example 1 except for using the compound B instead of the compound 9 as a host to form an emission layer.

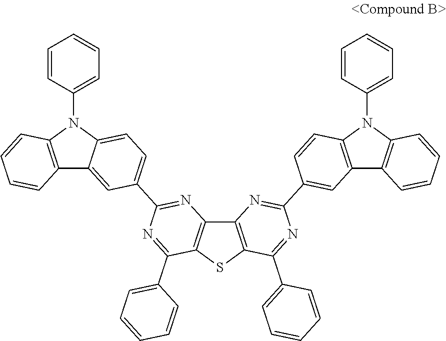 [SMILES: c1ccc(-c2nc(-c3ccc4c(c3)c3ccccc3n4-c3ccccc3)nc3c2sc2c(-c4ccccc4)nc(-c4ccc5c(c4)c4ccccc4n5-c4ccccc4)nc23)cc1]

Comparative Example 3

An organic light emitting device was manufactured according to the same method as Example 1 except for using the compound C instead of the compound 9 as a host to form an emission layer.

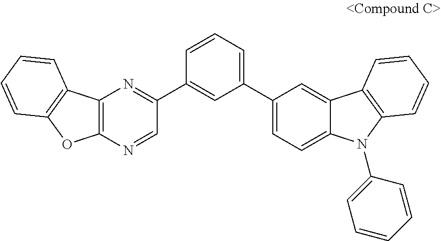 [SMILES: c1ccc(-n2c3ccccc3c3cc(-c4cccc(-c5cnc6oc7ccccc7c6n5)c4)ccc32)cc1]

Comparative Example 4

An organic light emitting device was manufactured according to the same method as Example 1 except for using the compound D instead of the compound 9 as a host to form an emission layer.

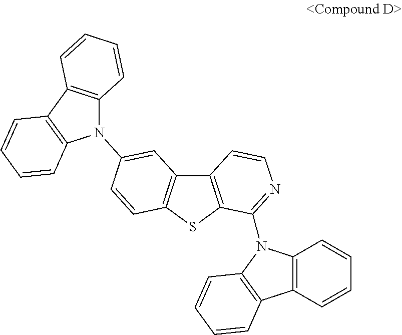 [SMILES: c1ccc2c(c1)c1ccccc1n2-c1ccc2sc3c(-n4c5ccccc5c5ccccc54)nccc3c2c1]

Evaluation Example 2: Evaluation of Characteristics of Organic Light Emitting Device

Driving voltages, efficiencies and luminance of the organic light emitting devices according to Examples 1 to 4 were evaluated using a current-voltage meter (Keithley 2400), and a luminance meter (Minolta Cs-1000A). Current efficiency was canculated using the measured luminance, current density, and voltage. The results are shown in the following Table 3.

TABLE 3

| | | | Driving | Current | Lumi- |
|---|---|---|---|---|---|
| | | | voltage | efficiency | nance |
| | Host | Dopant | (V) | (cd/A) | (cd/m²) |
| Example 1 | Compound 8 | Ir(ppy)₃ | 4.3 | 45 | 6000 |
| Example 2 | Compound 21 | Ir(ppy)₃ | 4.7 | 50 | 6000 |
| Example 3 | Compound 26 | Ir(ppy)₃ | 4.8 | 51 | 6000 |
| Example 4 | Compound 29 | Ir(ppy)₃ | 4.5 | 43 | 6000 |
| Comparative | Compound A | Ir(ppy)₃ | 5.0 | 38 | 6000 |
| Example 1 | | | | | |
| Comparative | Compound B | Ir(ppy)₃ | 5.1 | 29 | 6000 |
| Example 2 | | | | | |
| Comparative | Compound C | Ir(ppy)₃ | 4.8 | 34 | 6000 |
| Example 3 | | | | | |
| Comparative | Compound D | Ir(ppy)₃ | 4.8 | 31 | 6000 |
| Example 4 | | | | | |

As shown in Table 3, the organic light emitting devices according to Examples 1 to 4 showed excellent luminous efficiency compared with the organic light emitting devices according to Comparative Examples 1 to 4.

### DESCRIPTION OF SYMBOLS

10: organic light emitting device
11: first electrode
15: organic layer
19: second electrode

1. A condensed cyclic compound represented by the following Formula 1: 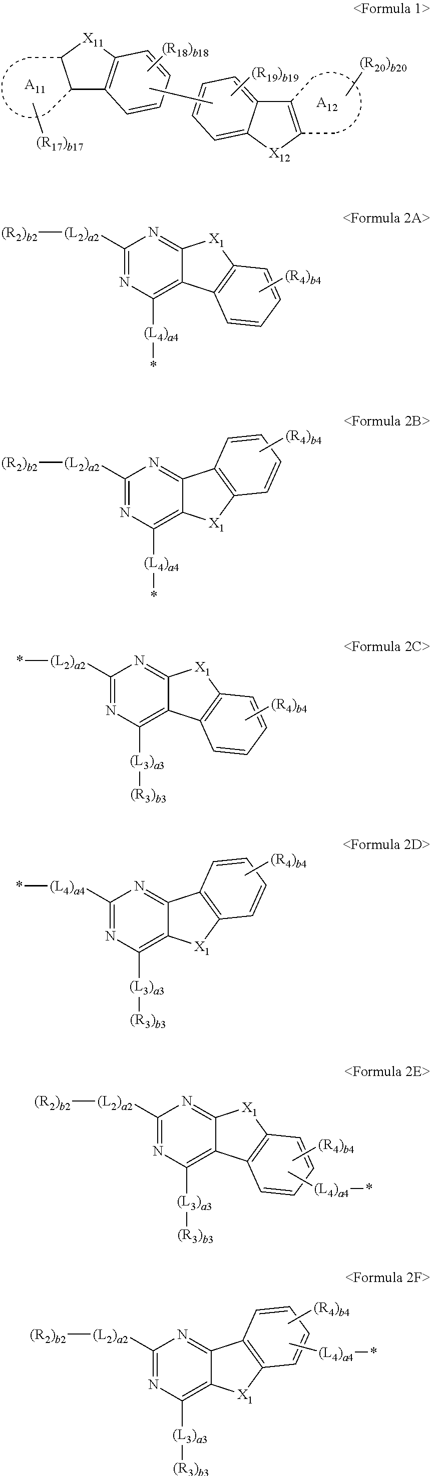
wherein, in the formulae 1, 2A, 2B, 2C, 2D, 2E, and 2F,
ring A₁₁ and ring A₁₂ are independently selected from benzene, naphthalene, pyridine, pyrimidine, pyrazine, pyridazine, and triazine;
X₁₁ is selected from N-[(L₁₁)ₐ₁₁-(R₁₁)_{b11}], S, O, S(═O), S(═O)₂, C(═O), C(R₁₃)(R₁₄), Si(R₁₃)(R₁₄), P(R₁₃), P(═O)(R₁₃), and C═N(R₁₃);
X₁₂ is selected from N-[(L₁₂)ₐ₁₂-(R₁₂)_{b12}], S, O, S(═O), S(═O)₂, C(═O), C(R₁₅)(R₁₆), Si(R₁₅)(R₁₆), P(R₁₅), P(═O)(R₁₅), and C═N(R₁₅);
X₁ is selected from N-[(L₁)ₐ₁-(R₁)_{b1}], S, O, S(═O), S(═O)₂, C(═O), Si(R₅)(R₆), P(R₅), P(═O)(R₅), and C═N(R₅);
L₁ to L₄, L₁₁ and L₁₂ are independently selected from a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₂-C₆₀ heteroarylene group, a substituted or unsubstituted divalent non-aromatic condensed polycyclic group, and a substituted or unsubstituted divalent non-aromatic heterocondensed polycyclic group;
a1 to a4, a11 and a12 are independently an integer selected from 0 to 3;
R₁ to R₆ and R₁₁ to R₂₀ are independently selected from hydrogen, deuterium, —F (a fluoro group), —Cl (a chloro group), —Br (a bromo group), —I (an iodo group), a hydroxyl group, a cyano group, a nitro group, an amino group, amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic heterocondensed polycyclic group, —N(Q₁)(Q₂), —Si(Q₃)(Q₄)(Q₅), —B(Q₆)(Q₇), and the group represented by the Formula 2A to the group represented by the Formula 2F, provided that the R₁ to R₆ are not the group represented by the Formula 2A to the group represented by the Formula 2F;
b1 to b4, b11, b12 and b16 to b20 are independently an integer selected from 1 to 3;
i) in Formula 1, when X₁₁ is N-[(L₁₁)ₐ₁₁-(R₁₁)_{b11}] and X₁₂ is not N-[(L₁₂)ₐ₁₂-(R₁₂)_{b12}], in Formula 1, at least one of b17 R₁₇'s, b11 R₁₁'s, b18 R₁₈'s, b19 R₁₉'s, and b20 R₂₀'s is selected from the group represented by the Formula 2A to the group represented by the Formula 2F;
ii) in Formula 1, when X₁₁ is not N-[(L₁₁)ₐ₁₁-(R₁₁)_{b11}] and X₁₂ is not N-[(L₁₂)ₐ₁₂-(R₁₂)_{b12}], in Formula 1, at least one of b17 R₁₇'s , b11 R₁₁'s, b18 R₁₈3 s, b19 R₁₉'s, and b20 R₂₀'s is selected from the group represented by the Formula 2A to the group represented by the Formula 2F;
iii) in Formula 1, when X₁₁ is N-[(L₁₁)ₐ₁₁-(R₁₁)_{b11}], and X₁₂ is N-[(L₁₂)ₐ₁₂-(R₁₂)_{b12}], in Formula 1, at least one of b17 R₁₇'s, b11 R₁₁'s, b18 R₁₈'s, b19 R₁₉'s, b12 R₁₂'s, and b20 R₂₀'s is selected from the group represented by the Formula 2A to the group represented by the Formula 2F;
at least one substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₂-C₆₀ heteroarylene group, the substituted divalent non-aromatic condensed polycyclic group, the substituted divalent non-aromatic heterocondensed polycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic heterocondensed polycyclic group is selected from
deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group and a C₁-C₆₀ alkoxy group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic heterocondensed polycyclic group, —N(Q₁₁)(Q₁₂), —Si(Q₁₃)(Q₁₄)(Q₁₅) and —B(Q₁₆)(Q₁₇);
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic heterocondensed polycyclic group, —N(Q₂₁)(Q₂₂), —Si(Q₂₃)(Q₂₄)(Q₂₅), and —B(Q₂₆)(Q₂₇); and
—N(Q₃₁)(Q₃₂), —Si(Q₃₃)(Q₃₄)(Q₃₅), and —B(Q₃₆)(Q₃₇);
wherein the Q₁ to Q₇, Q₁₁ to Q₁₇, Q₂₁ to Q₂₇ and Q₃₁ to Q₃₇ are independently selected from hydrogen, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group.

2. The condensed cyclic compound of claim 1, wherein the L₁ to L₄, L₁₁ and L₁₂ are independently selected from
a phenylene group, a pentalenylene group, an indenylene group, a naphthylene group, an azulenylene group, a heptalenylene group, an indacenylene group, an acenaphthylene group, a fluorenylene group, a spiro-fluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, a pentaphenylene group, hexacenylene group, a pyrrolylene group, an imidazolylene group, a pyrazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzooxazolylene group, a benzoimidazolylene group, a furanylene group, a benzofuranylene group, a thiophenylene group, a benzothiophenylene group, s thiazolylene group, an isothiazolylene group, a benzothiazolylene group, an isoxazolylene group, an oxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, an imidazopyrimidinylene group, and an imidazopyridinylene group; and
a phenylene group, a pentalenylene group, an indenylene group, a naphthylene group, an azulenylene group, a heptalenylene group, an indacenylene group, an acenaphthylene group, a fluorenylene group, a spiro-fluorenylene group, a phenalenylene group, a phenanthrenylene group, an anthracenylene group, a fluoranthrenylene group, a triphenylenylene group, a pyrenylene group, a chrysenylene group, a naphthacenylene group, a picenylene group, a perylenylene group, pentaphenylene group, a hexacenylene group, a pyrrolylene group, an imidazolylene group, a pyrazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzooxazolylene group, a benzoimidazolylene group, a furanylene group, a benzofuranylene group, a thiophenylene group, a benzothiophenylene group, a thiazolylene group, an isothiazolylene group, a benzothiazolylene group, an isoxazolylene group, an oxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, an imidazopyrimidinylene group and an imidazopyridinylene group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₆-C₂₀ aryl group, a C₂-C₆₀ heteroaryl group, a monovalent non-aromatic condensed polycyclic group, a monovalent non-aromatic heterocondensed ring group, and —Si(Q₃₃)(Q₃₄)(Q₃₅); and
wherein Q₃₃ to Q₃₅ are independently selected from hydrogen, a C₁-C₂₀ alkyl group, a C₁-C₂₀ alkoxy group, a phenyl group, a naphthyl group, an anthracenyl group, a pyrenyl group, a phenanthrenyl group, a fluorenyl group, a carbazolyl group, a benzocarbazolyl group, a dibenzocarbazolyl group, a pyridinyl group, a pyrimidinyl group, a pyrazinyl group, a pyridazinyl group, a triazinyl group, a quinolinyl group, an isoquinolinyl group, a phthalazinyl group, a quinoxalinyl group, a cinnolinyl group or a quinazolinyl group.
a1 to a4, a11 and a12 are independently 0 or 1.

3. The condensed cyclic compound of claim 1, wherein the R₁ to R₆ and
R₁₁ to R₂₀ are independently selected from
hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group and a C₁-C₂₀ alkoxy group;
a C₁-C₂₀ alkyl group and a C₁-C₂₀ alkoxy group substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof a sulfonic acid or a salt thereof, and a phosphoric acid or a salt thereof;
the following Formulae 4-1 to 4-31;
—Si(Q₃)(Q₄)(Q₅) (provided that the R₁₃ to R₁₆ and R₅ to R₆ are not —Si(Q₃)(Q₄)(Q₅)); and
the group represented by the Formula 2A to the group represented by the Formula 2F (provided that R₁ to R₆ are not the group represented by the Formula 2A to the group represented by the Formula 2F): 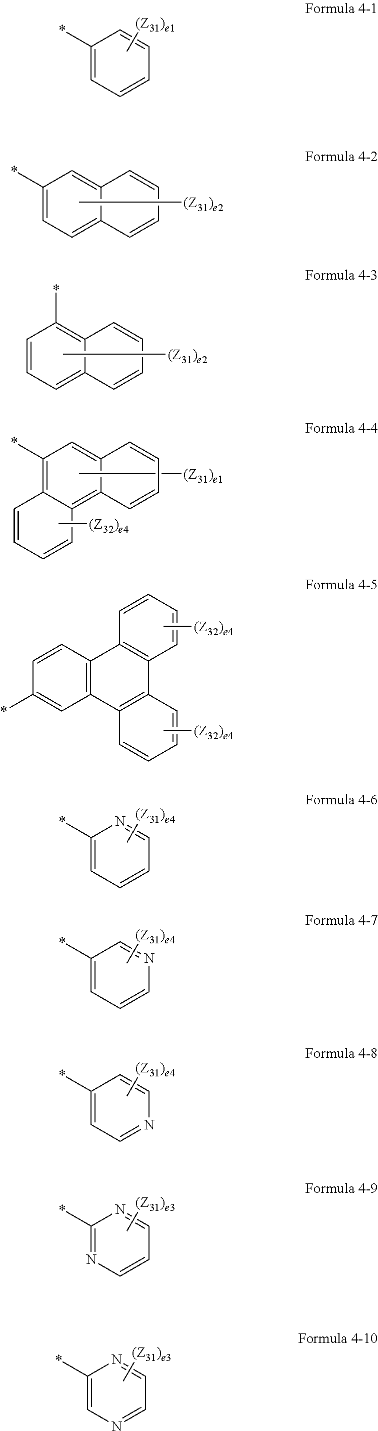 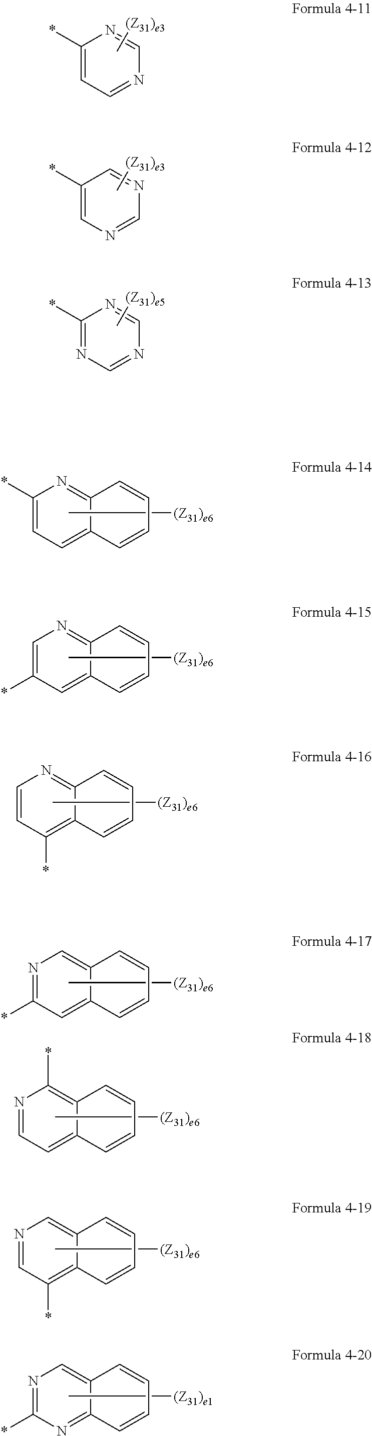 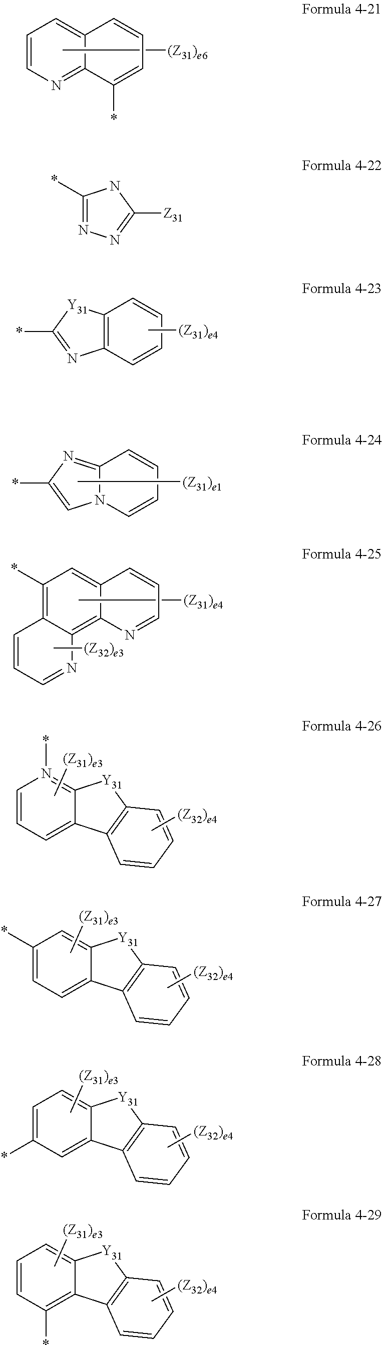 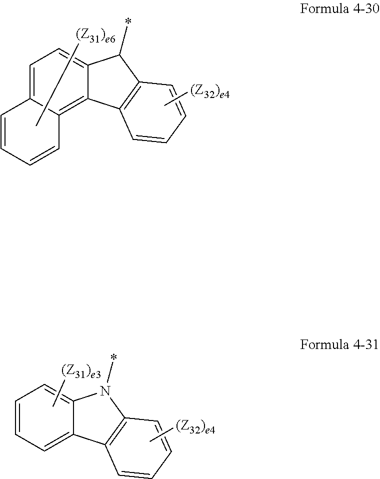
wherein, in Formulae 4-1 to 4-31,
Y₃₁ is O, S, C(Z₃₃)(Z₃₄), N(Z₃₅) or Si(Z₃₆)(Z₃₇) (provided that, in Formula 4-23, Y₃₁ is not NH);
Z₃₁ to Z₃₇ are independently selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, an anthracenyl group, a triphenylenyl group, a pyrenyl group, a phenanthrenyl group, a fluorenyl group, a chrysenyl group, a carbazolyl group, a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group, a quinoxalinyl group, a biphenyl group, and —Si(Q₃₃)(Q₃₄)(Q₃₅);
wherein the Q₃ to Q₅ and Q₃₃ to Q₃₅ are independently selected from hydrogen, a C₁-C₂₀ alkyl group, a C₁-C₂₀ alkoxy group, a phenyl group, a naphthyl group, an anthracenyl group, a triphenylenyl group, a pyrenyl group, a phenanthrenyl group, a fluorenyl group, a chrysenyl group, a carbazolyl group, a benzocarbazolyl group, a dibenzocarbazolyl group, a dibenzofuranyl group, a dibenzothiophenyl group, a pyridinyl group, a pyrimidinyl group, a triazinyl group, a quinolinyl group, an isoquinolinyl group, a quinazolinyl group and a quinoxalinyl group;
e1 is selected from integers of 1 to 5, e2 is selected from integers of 1 to 7, e3 is selected from integers of 1 to 3, e4 is selected from integers of 1 to 4, e5 is 1 or 2, e6 is selected from integers of 1 to 6, and * indicates a bonding site with a neighboring atom.

4. The condensed cyclic compound of claim 1, wherein the R₁ to R₆ and R₁₁ to R₂₀ are independently selected from
hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and a C₁-C₂₀ alkoxy group;
a C₁-C₂₀ alkyl group and a C₁-C₂₀ alkoxy group substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, and a phosphoric acid or a salt thereof;
the following Formula 5-1 to 5-63;
—Si(Q₃)(Q₄)(Q₅) (provided that the R₁₃ to R₁₆, R₅ and R₆ are not —Si(Q₃)(Q₄)(Q₅)); and
the group represented by the Formula 2A to the group represented by the Formula 2F (provided that the R₁ to R₆ are not the group represented by the Formula 2A to the group represented by the Formula 2F): 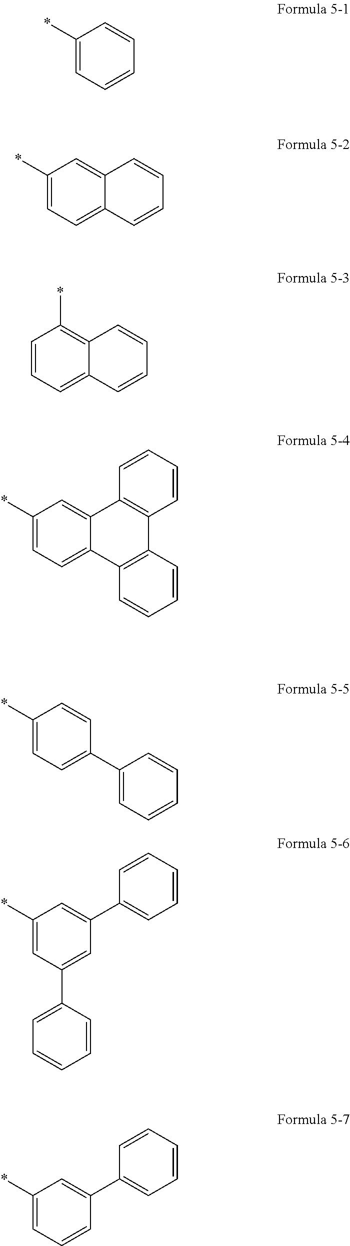 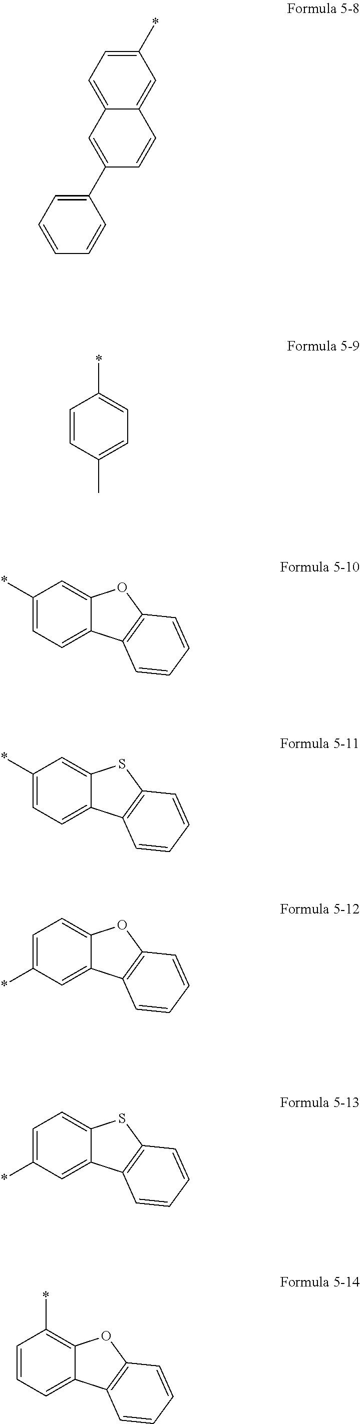 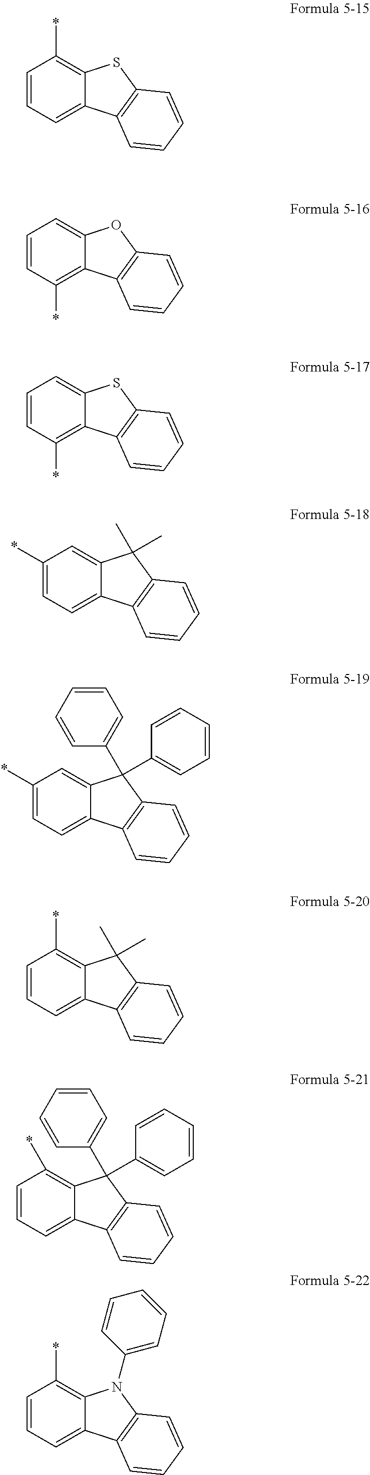 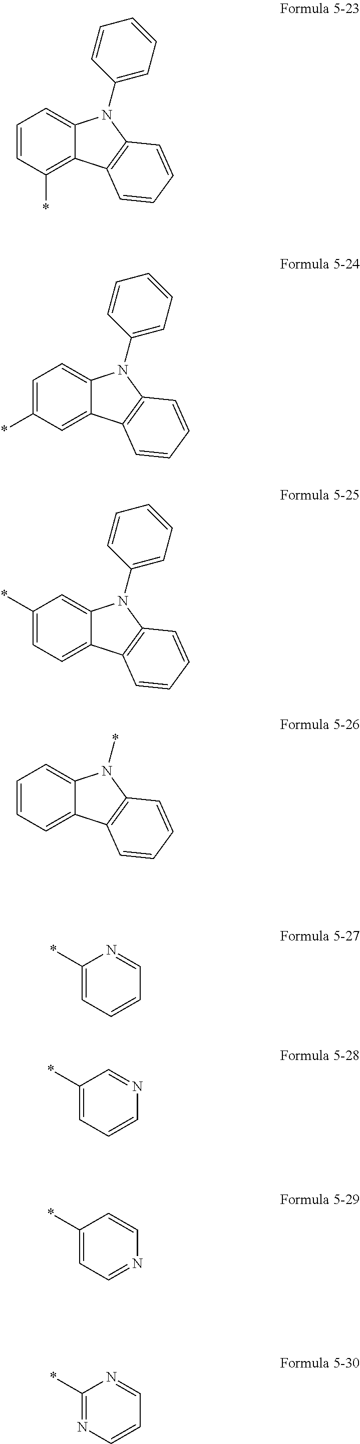 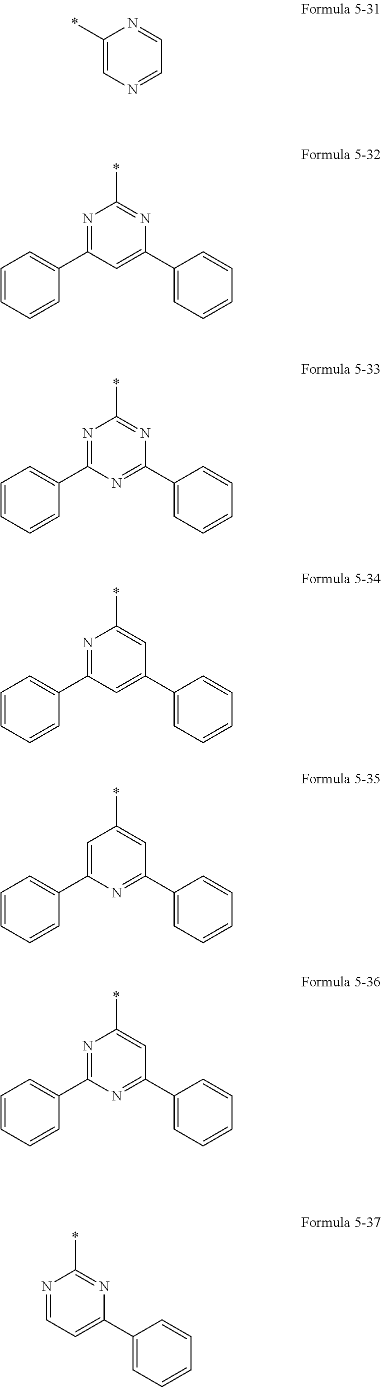 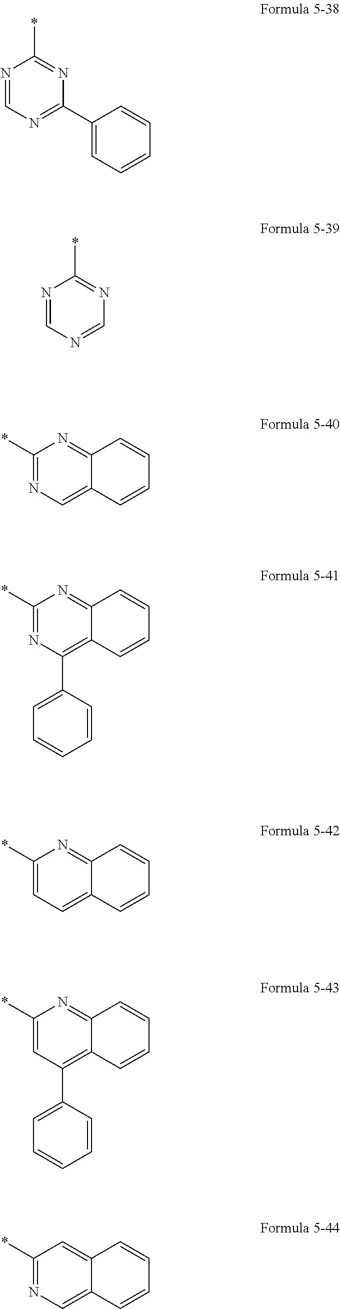 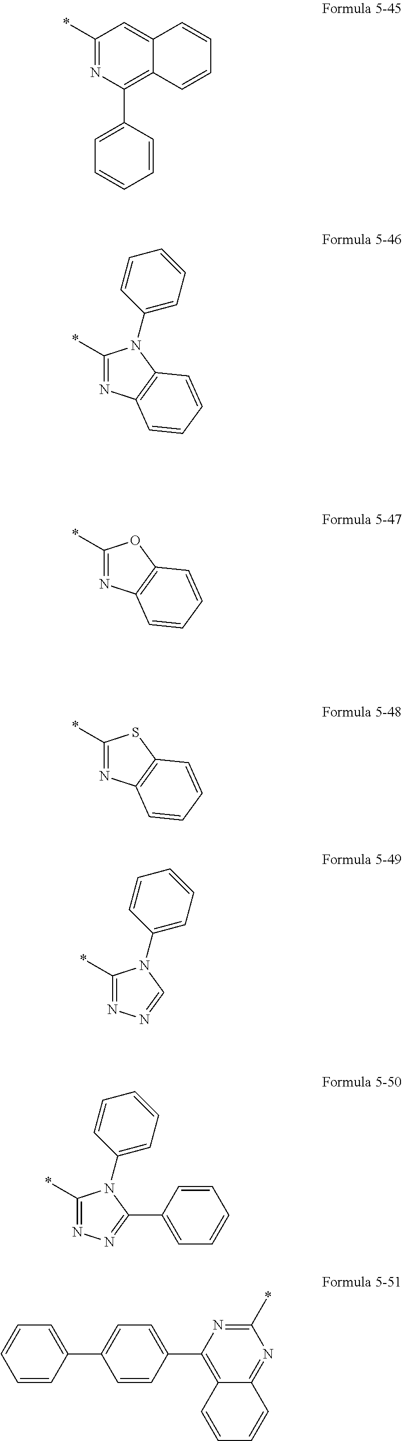 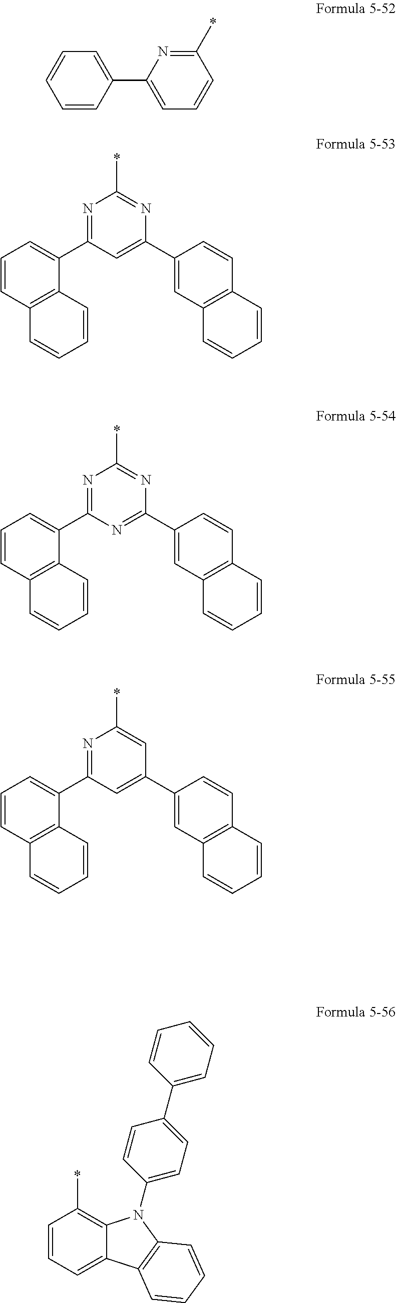 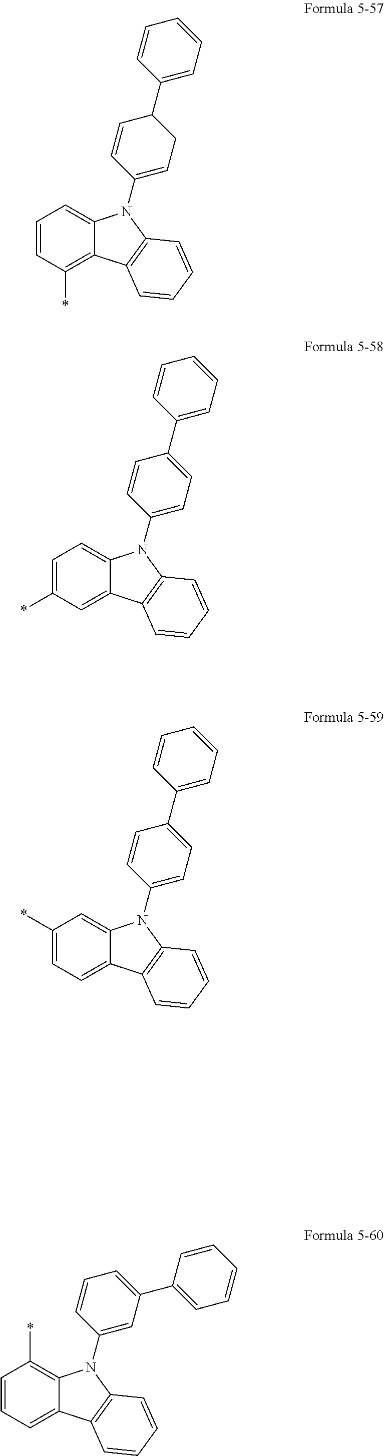 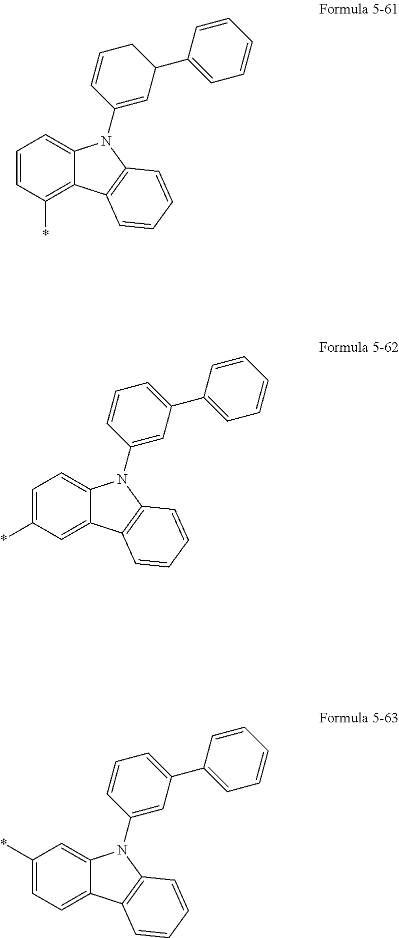
wherein, in Formulae 5-1 to 5-63, * indicates a bonding site with a neighboring atom.

5. The condensed cyclic compound of claim 1, wherein the R₁, R₁₁ and R₁₂ are independently selected from a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₂-C₂₀ heteroaryl group, a substituted or unsubstituted monovalent non-aromatic condensed polycyclic group, a substituted or unsubstituted monovalent non-aromatic heterocondensed polycyclic group, and the group represented by the Formula 2A to the group represented by the Formula 2F, provided that the R₁ is not the group represented by the Formula 2A to the group represented by the Formula 2F; and
the R₄ is selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and C₁-C₂₀ alkoxy group.

6. The condensed cyclic compound of claim 1, which is represented by one of the following Formulae 1-1 to 1-9: 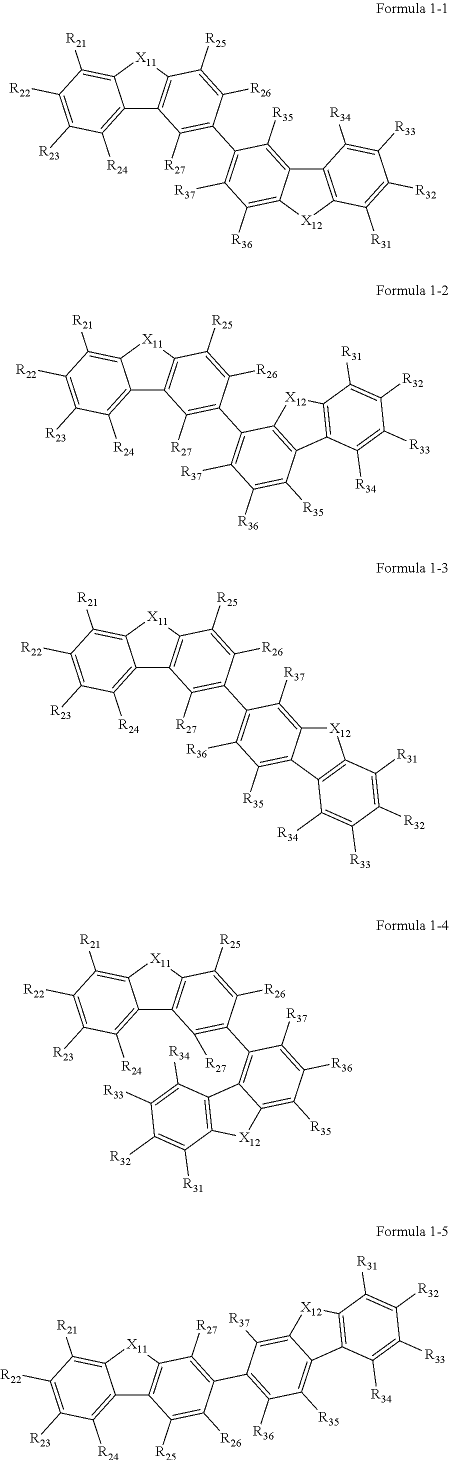 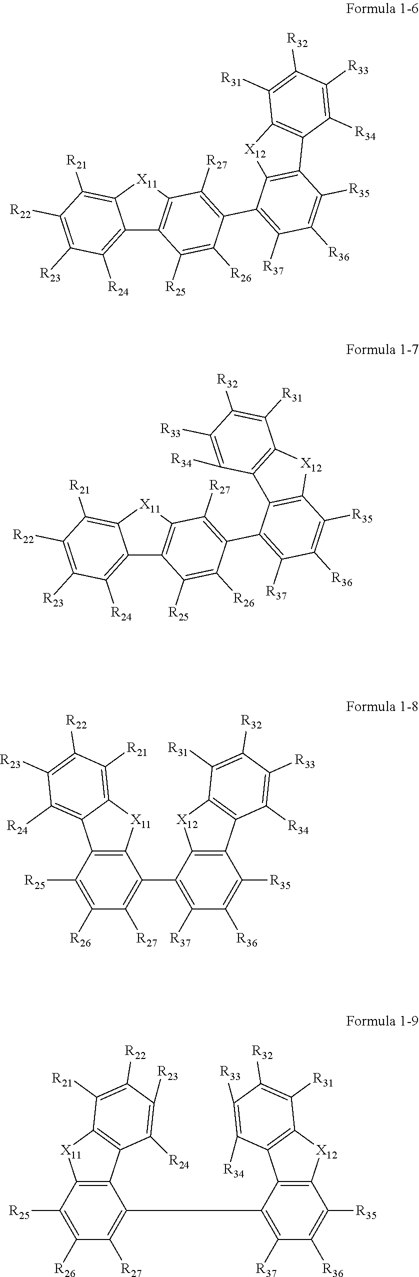
wherein, in Formulae 1-1 to 1-9, descriptions for X₁₁ and X₁₂ are the same as described in claim 1, descriptions for R₂₁ to R₂₄ are the same as R₁₇, descriptions for R₂₅ to R₂₇ are the same as R₁₈, descriptions for R₃₁ to R₃₄ are the same as R₂₀, and descriptions for R₃₅ to R₃₇ are the same as R₁₉.

7. The condensed cyclic compound of claim 1, which is one of the compounds 1 to 30:

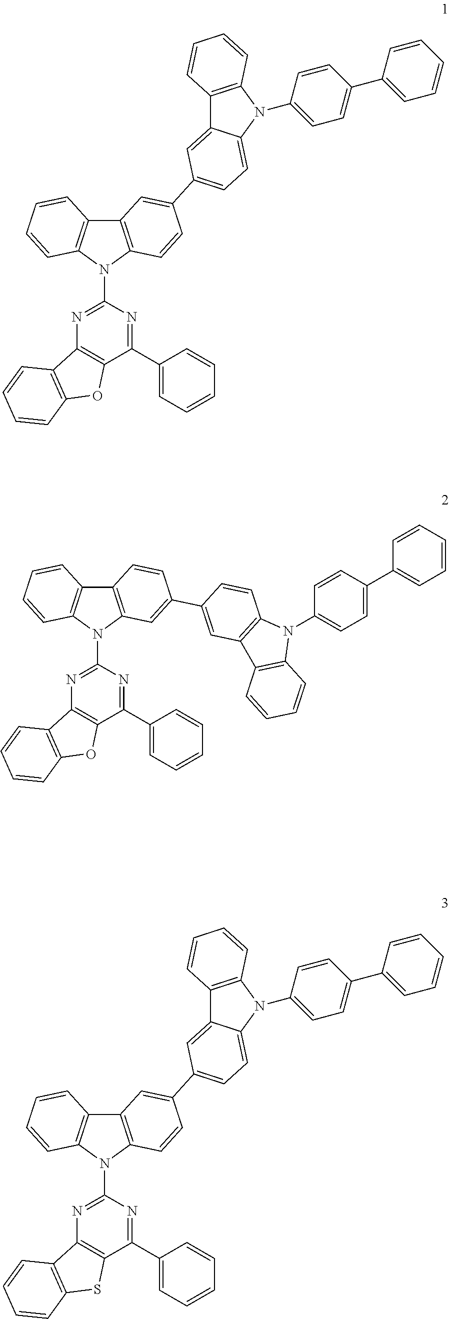 [SMILES: c1ccc(-c2ccc(-n3c4ccccc4c4cc(-c5ccc6c(c5)c5ccccc5n6-c5nc(-c6ccccc6)c6oc7ccccc7c6n5)ccc43)cc2)cc1 ; c1ccc(-c2ccc(-n3c4ccccc4c4cc(-c5ccc6c7ccccc7n(-c7nc(-c8ccccc8)c8oc9ccccc9c8n7)c6c5)ccc43)cc2)cc1 ; c1ccc(-c2ccc(-n3c4ccccc4c4cc(-c5ccc6c(c5)c5ccccc5n6-c5nc(-c6ccccc6)c6sc7ccccc7c6n5)ccc43)cc2)cc1]

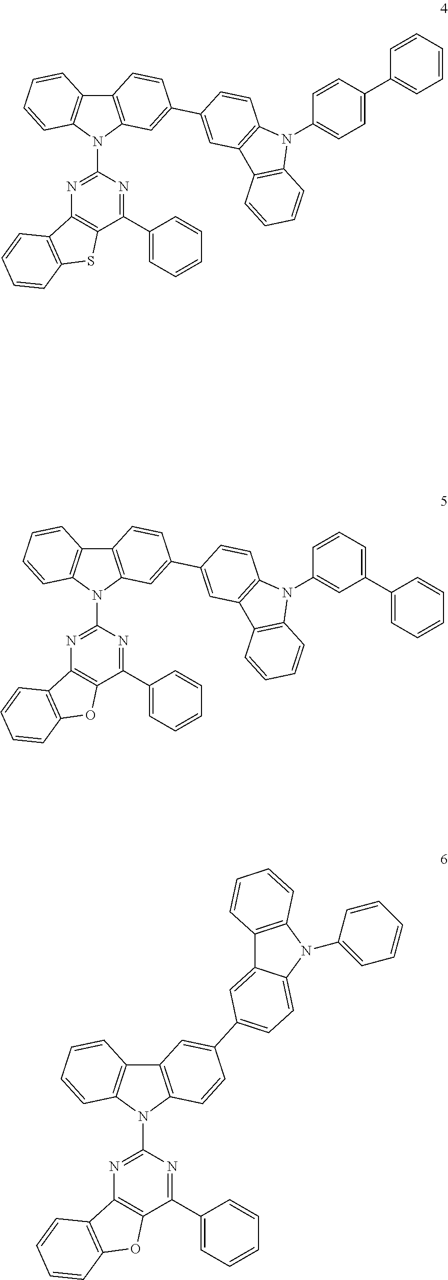 [SMILES: c1ccc(-c2ccc(-n3c4ccccc4c4cc(-c5ccc6c7ccccc7n(-c7nc(-c8ccccc8)c8sc9ccccc9c8n7)c6c5)ccc43)cc2)cc1 ; c1ccc(-c2cccc(-n3c4ccccc4c4cc(-c5ccc6c7ccccc7n(-c7nc(-c8ccccc8)c8oc9ccccc9c8n7)c6c5)ccc43)c2)cc1 ; c1ccc(-c2nc(-n3c4ccccc4c4cc(-c5ccc6c(c5)c5ccccc5n6-c5ccccc5)ccc43)nc3c2oc2ccccc23)cc1]

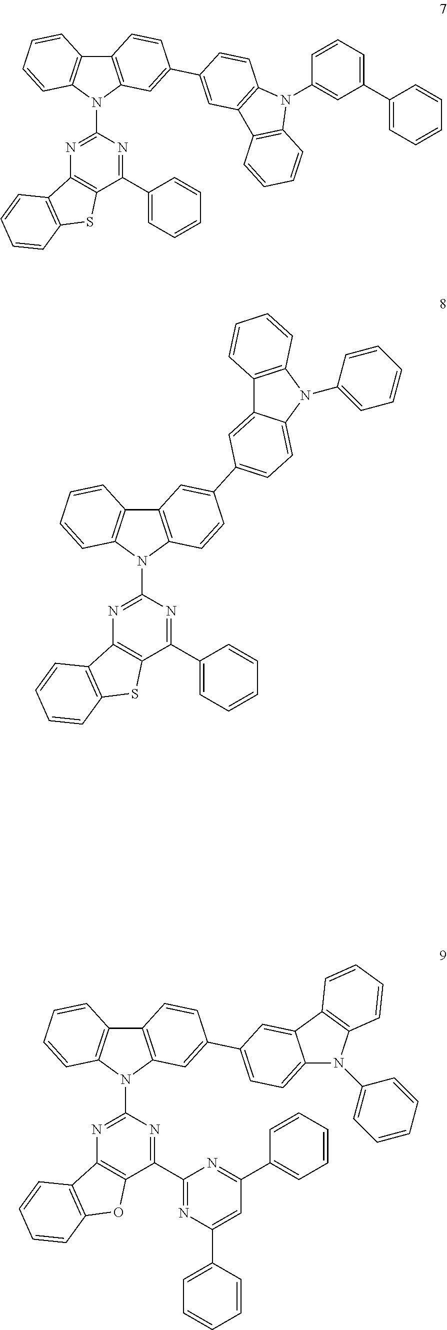 [SMILES: c1ccc(-c2cccc(-n3c4ccccc4c4cc(-c5ccc6c7ccccc7n(-c7nc(-c8ccccc8)c8sc9ccccc9c8n7)c6c5)ccc43)c2)cc1 ; c1ccc(-c2nc(-n3c4ccccc4c4cc(-c5ccc6c(c5)c5ccccc5n6-c5ccccc5)ccc43)nc3c2sc2ccccc23)cc1 ; c1ccc(-c2cc(-c3ccccc3)nc(-c3nc(-n4c5ccccc5c5ccc(-c6ccc7c(c6)c6ccccc6n7-c6ccccc6)cc54)nc4c3oc3ccccc34)n2)cc1]

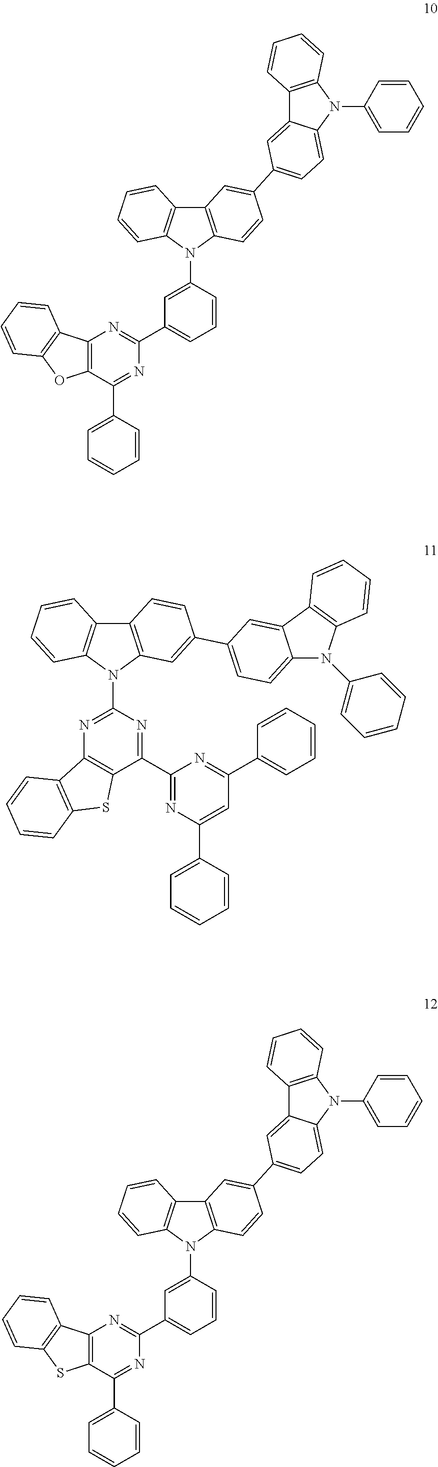 [SMILES: c1ccc(-c2nc(-c3cccc(-n4c5ccccc5c5cc(-c6ccc7c(c6)c6ccccc6n7-c6ccccc6)ccc54)c3)nc3c2oc2ccccc23)cc1 ; c1ccc(-c2cc(-c3ccccc3)nc(-c3nc(-n4c5ccccc5c5ccc(-c6ccc7c(c6)c6ccccc6n7-c6ccccc6)cc54)nc4c3sc3ccccc34)n2)cc1 ; c1ccc(-c2nc(-c3cccc(-n4c5ccccc5c5cc(-c6ccc7c(c6)c6ccccc6n7-c6ccccc6)ccc54)c3)nc3c2sc2ccccc23)cc1]

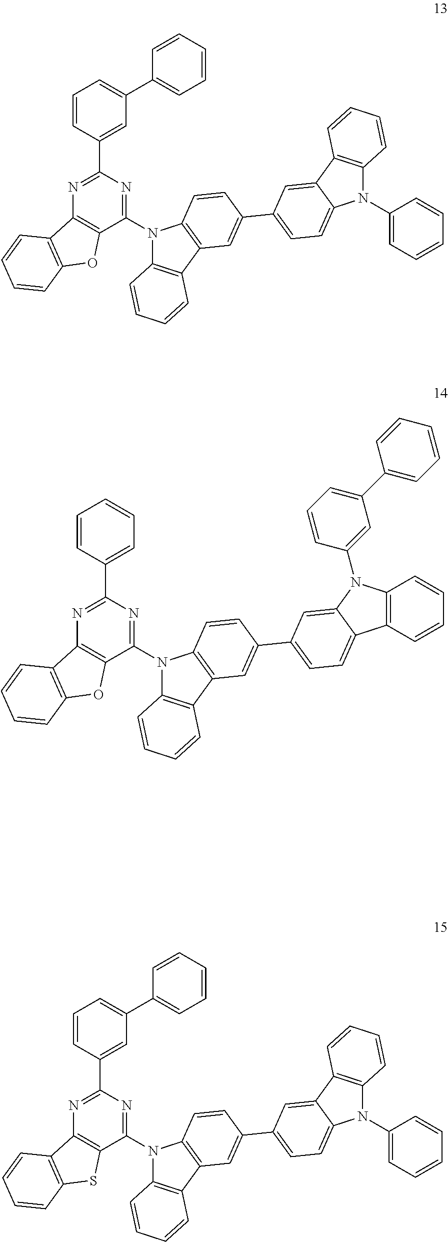 [SMILES: c1ccc(-c2cccc(-c3nc(-n4c5ccccc5c5cc(-c6ccc7c(c6)c6ccccc6n7-c6ccccc6)ccc54)c4oc5ccccc5c4n3)c2)cc1 ; c1ccc(-c2cccc(-n3c4ccccc4c4ccc(-c5ccc6c(c5)c5ccccc5n6-c5nc(-c6ccccc6)nc6c5oc5ccccc56)cc43)c2)cc1 ; c1ccc(-c2cccc(-c3nc(-n4c5ccccc5c5cc(-c6ccc7c(c6)c6ccccc6n7-c6ccccc6)ccc54)c4sc5ccccc5c4n3)c2)cc1]

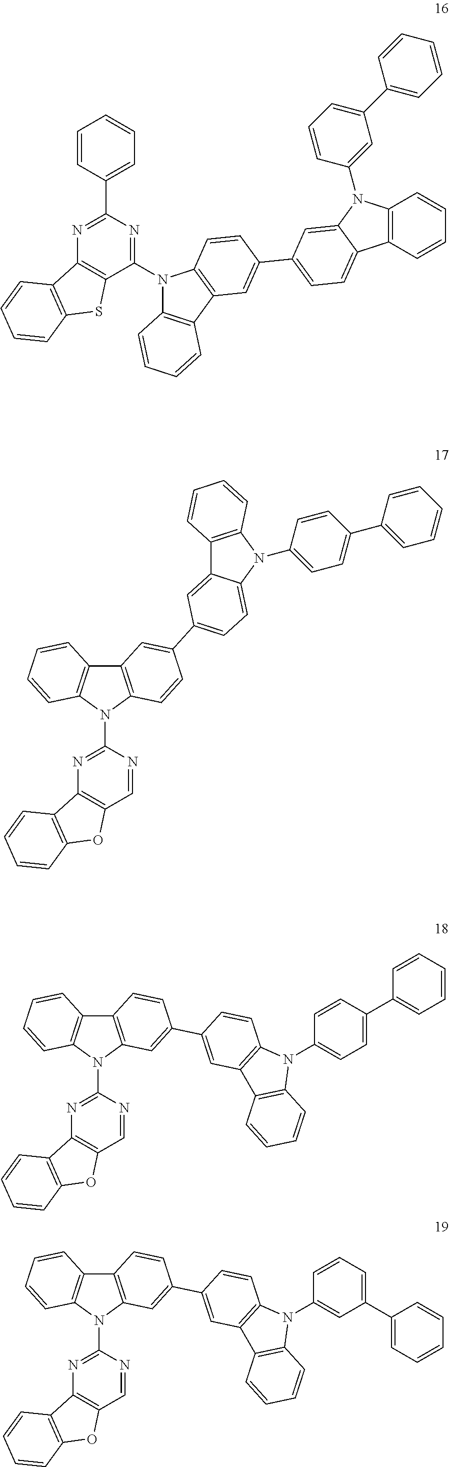 [SMILES: c1ccc(-c2cccc(-n3c4ccccc4c4ccc(-c5ccc6c(c5)c5ccccc5n6-c5nc(-c6ccccc6)nc6c5sc5ccccc56)cc43)c2)cc1 ; c1ccc(-c2ccc(-n3c4ccccc4c4cc(-c5ccc6c(c5)c5ccccc5n6-c5ncc6oc7ccccc7c6n5)ccc43)cc2)cc1 ; c1ccc(-c2ccc(-n3c4ccccc4c4cc(-c5ccc6c7ccccc7n(-c7ncc8oc9ccccc9c8n7)c6c5)ccc43)cc2)cc1 ; c1ccc(-c2cccc(-n3c4ccccc4c4cc(-c5ccc6c7ccccc7n(-c7ncc8oc9ccccc9c8n7)c6c5)ccc43)c2)cc1]

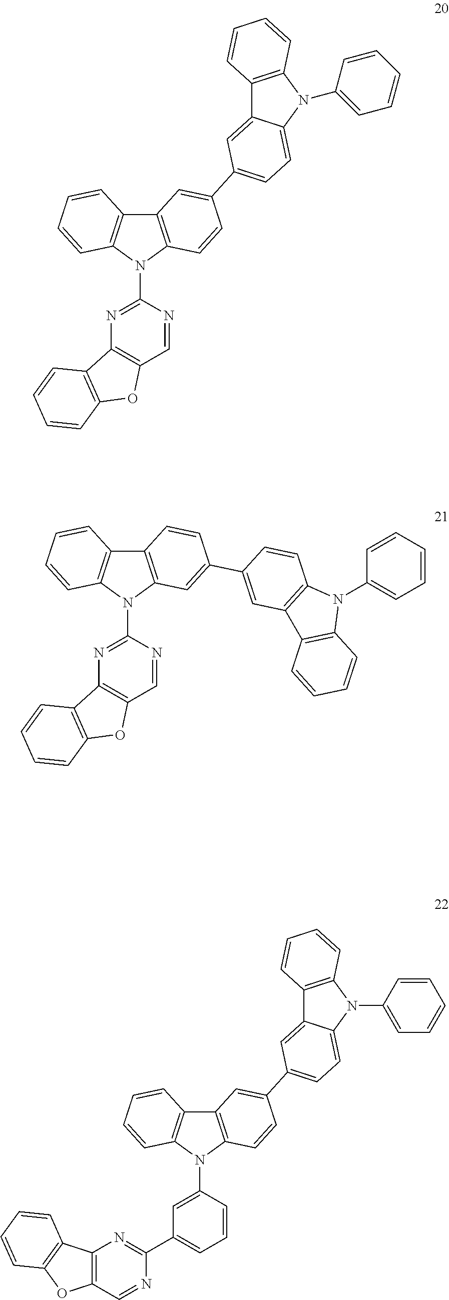 [SMILES: c1ccc(-n2c3ccccc3c3cc(-c4ccc5c(c4)c4ccccc4n5-c4ncc5oc6ccccc6c5n4)ccc32)cc1 ; c1ccc(-n2c3ccccc3c3cc(-c4ccc5c6ccccc6n(-c6ncc7oc8ccccc8c7n6)c5c4)ccc32)cc1 ; c1ccc(-n2c3ccccc3c3cc(-c4ccc5c(c4)c4ccccc4n5-c4cccc(-c5ncc6oc7ccccc7c6n5)c4)ccc32)cc1]

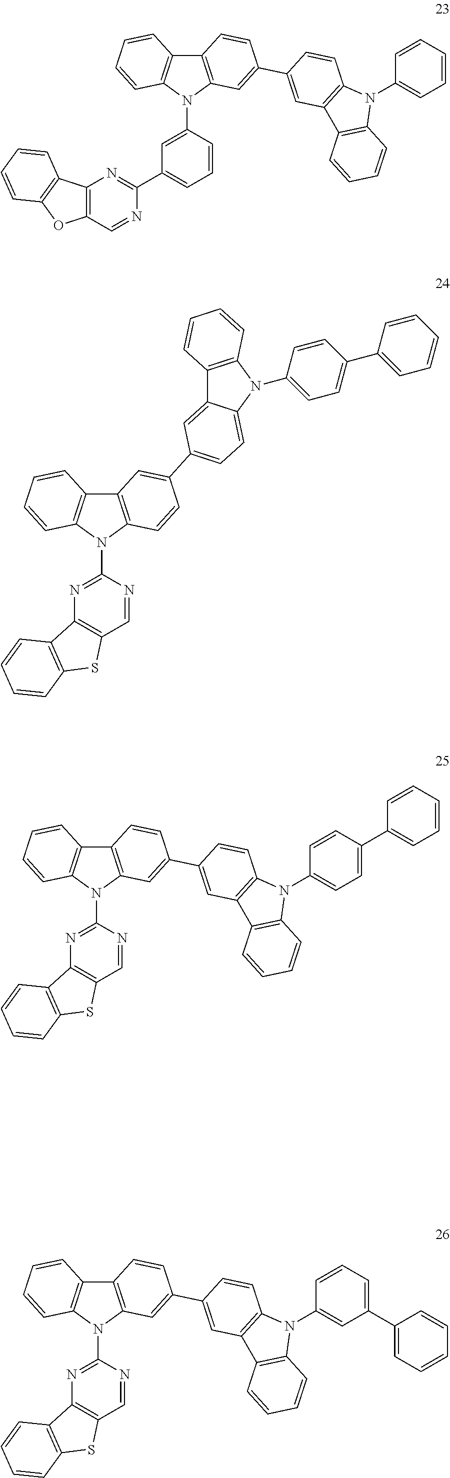 [SMILES: c1ccc(-n2c3ccccc3c3cc(-c4ccc5c6ccccc6n(-c6cccc(-c7ncc8oc9ccccc9c8n7)c6)c5c4)ccc32)cc1 ; c1ccc(-c2ccc(-n3c4ccccc4c4cc(-c5ccc6c(c5)c5ccccc5n6-c5ncc6sc7ccccc7c6n5)ccc43)cc2)cc1 ; c1ccc(-c2ccc(-n3c4ccccc4c4cc(-c5ccc6c7ccccc7n(-c7ncc8sc9ccccc9c8n7)c6c5)ccc43)cc2)cc1 ; c1ccc(-c2cccc(-n3c4ccccc4c4cc(-c5ccc6c7ccccc7n(-c7ncc8sc9ccccc9c8n7)c6c5)ccc43)c2)cc1]

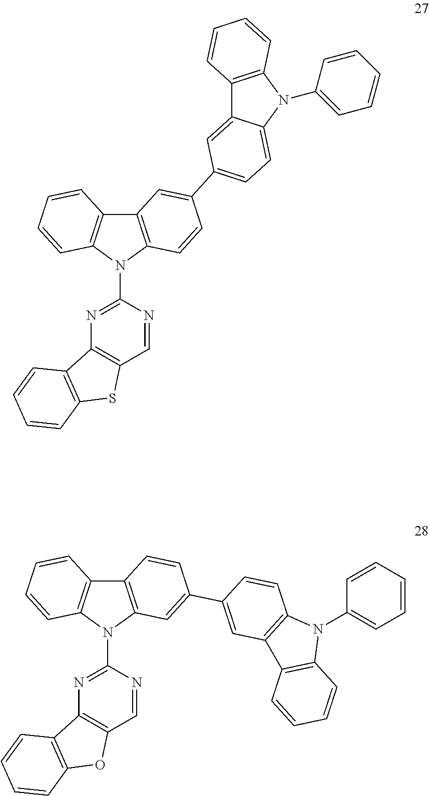 [SMILES: c1ccc(-n2c3ccccc3c3cc(-c4ccc5c(c4)c4ccccc4n5-c4ncc5sc6ccccc6c5n4)ccc32)cc1 ; c1ccc(-n2c3ccccc3c3cc(-c4ccc5c6ccccc6n(-c6ncc7oc8ccccc8c7n6)c5c4)ccc32)cc1]

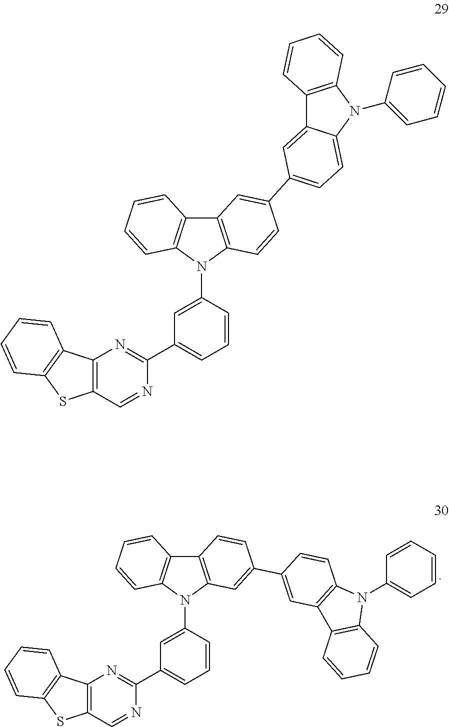 [SMILES: c1ccc(-n2c3ccccc3c3cc(-c4ccc5c(c4)c4ccccc4n5-c4cccc(-c5ncc6sc7ccccc7c6n5)c4)ccc32)cc1 ; c1ccc(-n2c3ccccc3c3cc(-c4ccc5c6ccccc6n(-c6cccc(-c7ncc8sc9ccccc9c8n7)c6)c5c4)ccc32)cc1]

8. An organic light emitting device comprising
a first electrode; a second electrode facing the first electrode; and an organic layer comprising an emission layer interposed between the first electrode and the second electrode, and the organic layer comprises at least one condensed cyclic compound of claim 1.

9. The organic light emitting device of claim 8, wherein the condensed cyclic compound is present in the emission layer.

10. The organic light emitting device of claim 8, wherein the emission layer comprises a first host, a second host and a dopant,
the first host and the second host are different from each other,
the first host comprises the condensed cyclic compound, and
the second host comprises at least one of a first compound represented by the following Formula 41 and a second compound represented by the following Formula 61: 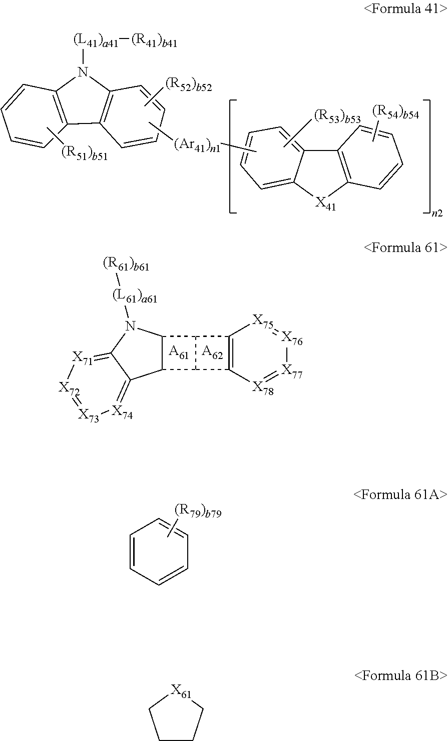
wherein, in formulae 41 and 61,
X₄₁ is selected from N-[(L₄₂)ₐ₄₂-(R₄₂)_{b42}], S, O, S(═O), S(═O)₂, C(═O), C(R₄₃)(R₄₄), Si(R₄₃)(R₄₄), P(R₄₃), P(═O)R₄₃, and C═N(R₄₃);
in Formula 61, the ring A₆₁ is represented by the Formula 61A;
in Formula 61, the ring A₆₂ is represented by the Formula 61B;
X₆₁ is selected from N-[(L₆₂)ₐ₆₂-(R₆₂)_{b62}], S, O, S(═O), S(═O)₂, C(═O), C(R₆₃)(R₆₄), Si(R₆₃)(R₆₄), P(R₆₃), P(═O)R₆₃, and C═N(R₆₃);
X₇₁ is C(R₇₁) or N, X₇₂ is C R₇₂ or N, X₇₃ is C(R₇₃) or N, X₇₄ is C(R₇₄) or N, X₇₅ is C(R₇₅) or N, X₇₆ is C(R₇₆) or N, X₇₇ is C(R₇₇) or N, X₇₈ is C(R₇₈) or N;
Ar₄₁, L₄₁, L₄₂, L₆₁ and L₆₂ are independently selected from a substituted or unsubstituted C₃-C₁₀ cycloalkylene group, a substituted or unsubstituted C₂-C₁₀ heterocycloalkylene group, a substituted or unsubstituted C₃-C₁₀ cycloalkenylene group, a substituted or unsubstituted C₂-C₁₀heterocycloalkenylene group, a substituted or unsubstituted C₆-C₆₀ arylene group, a substituted or unsubstituted C₂-C₆₀ heteroarylene group, a substituted or unsubstituted divalent non-aromatic condensed polycyclic group, and a substituted or unsubstituted divalent non-aromatic heterocondensed polycyclic group;
n1 and n2 are independently selected from integers of 0 to 3;
a41, a42, a61 and a62 are independently selected from integers of 0 to 3;
R₄₁ to R₄₃, R₅₁ to R₅₄, R₆₁ to R₆₄ and R₇₁ to R₇₉ are independently selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic heterocondensed polycyclic group, —N(Q₁)(Q₂), —Si(Q₃)(Q₄)(Q₅), and —B(Q₆)(Q₇); and
b41, b42, b51 to b54, b61, b62 and b79 are independently selected from integers of 1 to 3.
at least one substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₂-C₆₀ heteroarylene group, the substituted divalent non-aromatic condensed polycyclic group, the substituted divalent non-aromatic heterocondensed polycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic heterocondensed polycyclic group is selected from
deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group and a C₁-C₆₀ alkoxy group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic heterocondensed polycyclic group, —N(Q₁₁)(Q₁₂), —Si(Q₁₃)(Q₁₄)(Q₁₅) and —B(Q₁₆)(Q₁₇);
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group, each of which is substituted with at least one of deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic heterocondensed polycyclic group, —N(Q₂₁)(Q₂₂), —Si(Q₂₃)(Q₂₄)(Q₂₅), and —B(Q₂₆)(Q₂₇); and
—N(Q₃₁)(Q₃₂), —Si(Q₃₃)(Q₃₄)(Q₃₅), and —B(Q₃₆)(Q₃₇);
wherein the Q₁ to Q₇, Q₁₁ to Q₁₇, Q₂₁ to Q₂₇ and Q₃₁ to Q₃₇ are independently selected from hydrogen, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic heterocondensed polycyclic group.
